(19) United States
(12) Patent Application Publication
(10) Pub. No.: US 2012/0015297 A1
(43) Pub. Date: Jan. 19, 2012

(54) RESIST COMPOSITION, METHOD OF FORMING RESIST PATTERN, NOVEL COMPOUND, AND ACID GENERATOR

(75) Inventors: Yoshitaka KOMURO, Kawasaki-shi (JP); Yoshiyuki Utsumi, Kawasaki-shi (JP)

(73) Assignee: TOKYO OHKA KOGYO.CO., LTD., Kawasaki-shi (JP)

(21) Appl. No.: 13/177,859

(22) Filed: Jul. 7, 2011

(30) Foreign Application Priority Data
Jul. 15, 2010 (JP) P2010-161090

(51) Int. Cl. G03F 7/027; C07D 333/46; G03F 7/20

(52) U.S. Cl. 430/283.1; 430/281.1; 430/325; 549/79; 549/31; 546/280.4; 549/60

(57) ABSTRACT

A compound represented by general formula (b1); an acid generator including the compound; and a resist composition including a base component (A) which exhibits changed solubility in an alkali developing solution under action of acid and an acid-generator component (B) which generates acid upon exposure, the acid-generator component (B) including an acid generator (B1) including a compound represented by general formula (b1), wherein R¹ represents a hydrogen atom, a linear, branched or cyclic alkyl group of 1 to 10 carbon atoms or a heterocyclic group of 1 to 10 carbon atoms; R² represents a linear or branched alkyl group of 1 to 10 carbon atoms; x represents an integer of 0 to 6; n represents an integer of 0 to 3; and X⁻ represents an anion.

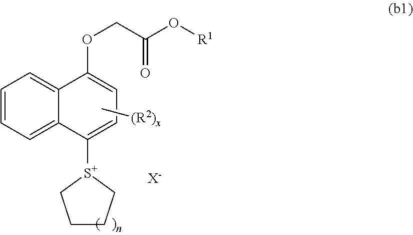

### TECHNICAL FIELD

The present invention relates to a resist composition, a method of forming a resist pattern using the same, a novel compound useful as an acid generator for a resist composition, and an acid generator.

Priority is claimed on Japanese Patent Application No. 2010-161090, filed Jul. 15, 2010, the content of which is incorporated herein by reference.

### BACKGROUND ART

In lithography techniques, for example, a resist film composed of a resist material is formed on a substrate, and the resist film is subjected to selective exposure of radial rays such as light or electron beam through a mask having a predetermined pattern, followed by development, thereby forming a resist pattern having a predetermined shape on the resist film. A resist material in which the exposed portions become soluble in a developing solution is called a positive-type, and a resist material in which the exposed portions become insoluble in a developing solution is called a negative-type.

In recent years, in the production of semiconductor elements and liquid crystal display elements, advances in lithography techniques have lead to rapid progress in the field of pattern miniaturization.

Typically, these miniaturization techniques involve shortening the wavelength of the exposure light source. Conventionally, ultraviolet radiation typified by g-line and i-line radiation has been used, but nowadays KrF excimer lasers and ArF excimer lasers are starting to be introduced in mass production. Furthermore, research is also being conducted into lithography techniques that use an exposure light source having a wavelength shorter than these excimer lasers, such as F2 excimer lasers, electron beam, extreme ultraviolet radiation (EUV), and X ray.

Resist materials for use with these types of exposure light sources require lithography properties such as a high resolution capable of reproducing patterns of minute dimensions, and a high level of sensitivity to these types of exposure light sources.

As a resist material that satisfies these conditions, a chemically amplified composition is used, which includes a base material component that exhibits a changed solubility in an alkali developing solution under the action of acid and an acid-generator component that generates acid upon exposure.

For example, a chemically amplified positive resist contains, as a base component (base resin), a resin which exhibits increased solubility in an alkali developing solution under action of acid, and an acid generator is typically used. If the resist film formed using the resist composition is selectively exposed during formation of a resist pattern, then within the exposed portions, acid is generated from the acid-generator component, and the action of this acid causes an increase in the solubility of the resin component in an alkali developing solution, making the exposed portions soluble in the alkali developing solution.

Currently, resins that contain structural units derived from (meth)acrylate esters within the main chain (acrylic resins) are now widely used as base resins for resists that use ArF excimer laser lithography, as they exhibit excellent transparency in the vicinity of 193 nm (for example, see Patent Document 1).

Here, the term “(meth)acrylic acid” is a generic term that includes either or both of acrylic acid having a hydrogen atom bonded to the α-position and methacrylic acid having a methyl group bonded to the α-position. The term “(meth)acrylate ester” is a generic term that includes either or both of the acrylate ester having a hydrogen atom bonded to the α-position and the methacrylate ester having a methyl group bonded to the α-position. The term “(meth)acrylate” is a generic term that includes either or both of the acrylate having a hydrogen atom bonded to the α-position and the methacrylate having a methyl group bonded to the α-position.

On the other hand, as acid generators usable in a chemically amplified resist composition, various types have been proposed including, for example, onium salt acid generators such as iodonium salts and sulfonium salts; oxime sulfonate acid generators; diazomethane acid generators; nitrobenzylsulfonate acid generators; iminosulfonate acid generators; and disulfone acid generators.

### DOCUMENTS OF RELATED ART

### Patent Document

[Patent Document 1] Japanese Unexamined Patent Application, First Publication No. 2003-241385
[Patent Document 2] Japanese Unexamined Patent Application, First Publication No. 2002-229192

### SUMMARY OF THE INVENTION

As the miniaturization of resist pattern progresses, for example, lithography using electron beam or EUV is aiming the formation of a fine pattern having a size of several tens of nanometers. As the size of the resist pattern becomes smaller, further improvement in the resolution of resist materials has been demanded while maintaining excellent lithography properties and capability of forming a resist pattern with excellent shape.

In response to such demands, Patent Document 2 describes a compound which improves the transparency of the resist composition. However, the acid generator having a naphthalene ring described in Patent Document 2 has room for further improving the lithography properties. However, such a resist material is essentially unknown in the art.

The present invention takes the above circumstances into consideration, with an object of providing a compound useful as an acid generator for a resist composition, an acid generator including the compound, a resist composition containing the acid generator, and a method of forming a resist pattern using the resist composition.

A first aspect of the present invention for solving the aforementioned problems is a resist composition including a base component (A) which exhibits changed solubility in an alkali developing solution under action of acid and an acid-generator component (B) which generates acid upon exposure,

the acid-generator component (B) including an acid generator (B1) including a compound represented by general formula (b1) shown below.

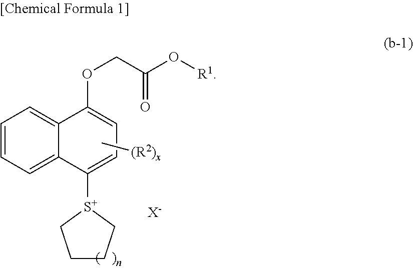

In the formula, R¹ represents a hydrogen atom, a linear, branched or cyclic alkyl group of 1 to 10 carbon atoms or a heterocyclic group of 1 to 10 carbon atoms; R² represents a linear or branched alkyl group of 1 to 10 carbon atoms; x represents an integer of 0 to 6; n represents an integer of 0 to 3; and X⁻ represents an anion.

A second aspect of the present invention is a method of forming a resist pattern, including forming a resist film on a substrate using a resist composition according to the first aspect, subjecting the resist film to exposure, and subjecting the resist film to alkali developing to form a resist pattern.

A third aspect of the present invention is a compound represented by general formula (b1) shown above.

A fourth aspect of the present invention is an acid generator including the compound of the third aspect of the present invention.

In the present description and claims, the term “structural unit” refers to a monomer unit that contributes to the formation of a polymeric compound (polymer, copolymer).

The term “exposure” is used as a general concept that includes irradiation with any form of radiation.

The term “alkyl group” includes linear, branched or cyclic, monovalent saturated hydrocarbon, unless otherwise specified.

A “lower alkyl group” is an alkyl group of 1 to 5 carbon atoms.

The term “acid dissociable group” refers to an organic group that is dissociable by the action of an acid.

According to the present invention, there are provided novel compound preferable as an acid generator for a resist composition, an acid generator including the compound, a resist composition including the acid generator, and a method of forming a resist pattern using the resist composition.

### DETAILED DESCRIPTION OF THE INVENTION

Hereafter, the present invention will be described in detail.

<<Compound According to Third Aspect>>

Firstly, the compound according to the third aspect of the present invention will be described. The compound according to the third aspect of the present invention is represented by the aforementioned general formula (b1).

In general formula (b1), R¹ represents a hydrogen atom, a linear, branched or cyclic alkyl group of 1 to 10 carbon atoms or a hetero cyclic group of 1 to 10 carbon atoms.

The linear or branched alkyl group of 1 to 10 carbon atoms for R¹ is not particularly limited. In terms of superiority in resolution, the linear or branched alkyl group preferably has 1 to 5 carbon atoms. Specific examples include a methyl group, an ethyl group, an n-propyl group, an isopropyl group, an n-butyl group, an isobutyl group, an n-pentyl group, a hexyl group, a nonyl group and a decyl group. When the compound is used as the component (B1) in the resist composition according to the first aspect of the present invention, a methyl group is preferable because it is excellent in resolution and can be synthesized at a low cost.

As the cyclic group of 1 to 10 carbon atoms for R¹, an adamantyl group, a norbornyl group, an isobornyl group or a tricyclodecyl group is preferable, and an adamantyl group is particularly desirable.

The heterocyclic group of 1 to 10 carbon atoms for R¹ is a monovalent cyclic group in which the ring is constituted of carbon atom and hetero atom (such as a nitrogen atom, an oxygen atom or a sulfur atom). The cyclic group is preferably a polycyclic group. As the heterocyclic group of 1 to 10 carbon atoms for R¹, an —SO₂— containing cyclic group represented by the formula shown below or a lactone-containing cyclic group is preferable.

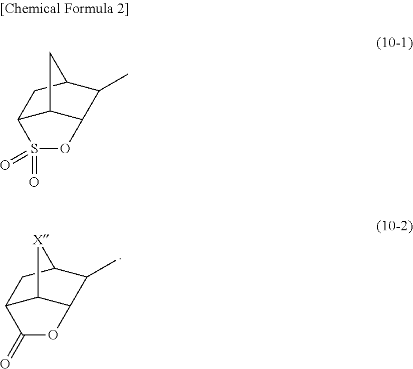

In the formula, X″ represents CH₂ or O.

In general formula (b1), R² represents a linear or branched alkyl group of 1 to 10 carbon atoms.

The alkyl group for R² is not particularly limited as long as it is a linear or branched alkyl group of 1 to 10 carbon atoms. In terms of superiority in resolution, the linear or branched alkyl group preferably has 1 to 5 carbon atoms. Specific examples thereof include a methyl group, an ethyl group, an n-propyl group, an isopropyl group, an n-butyl group, an isobutyl group, an n-pentyl group, a hexyl group, a nonyl group, and a decyl group, and a methyl group is most preferable because it is excellent in resolution and can be synthesized at a low cost.

In general formula (b1), x represents an integer of 0 to 6, and is preferably 0.

In general formula (b1), n represents an integer of 0 to 3, and is preferably 1.

The compound according to the third aspect of the present invention is preferably represented by general formula (b1-1) shown below.

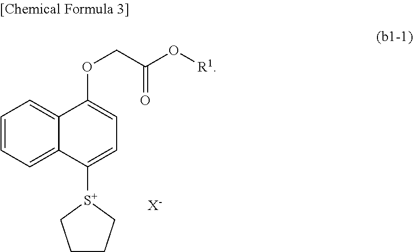

In the formula, R¹ represents a hydrogen atom, a linear, branched or cyclic alkyl group of 1 to 10 carbon atoms or a heterocyclic group of 1 to 10 carbon atoms; and X⁻ represents an anion.

Preferable examples of the cation moiety of the compound represented by general formula (b1-1) are shown below.

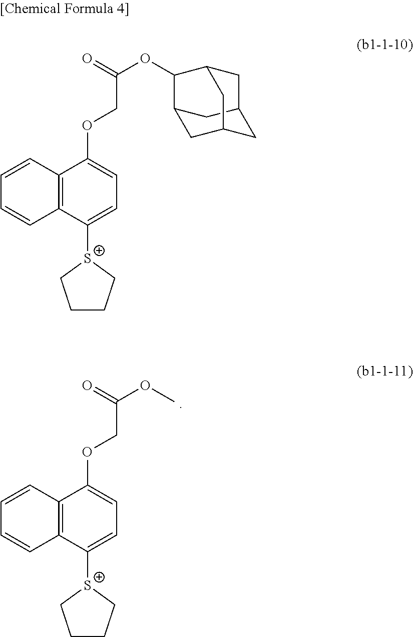

In general formula (b1), X⁻ represents an anion. X⁻ is not particularly limited, and is preferably at least one anion selected from the group consisting of compounds represented by any one of general formulas (1) to (4) shown below.

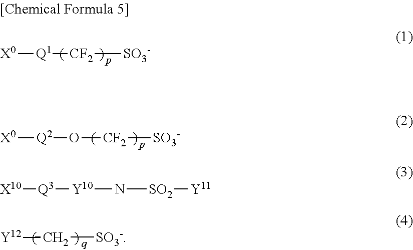

In the formulas, each X⁰ independently represents a hydrocarbon group of 1 to 30 carbon atoms which may have a substituent; Q¹ represents a single bond or a divalent linking group containing a carbonyl group; each p independently represents an integer of 0 to 3; Q² represents a single bond or an alkylene group; X¹⁰ represents a hydrocarbon group of 1 to 30 carbon atoms which may have a substituent; Q³ represents a single bond or a divalent linking group; Y¹⁰ represents —C(═O)— or —SO₂—; Y¹¹ represents an alkyl group of 1 to 10 carbon atoms which may have a substituent or a fluorinated alkyl group of 1 to 10 carbon atoms which may have a substituent; Y¹² represents a cyclic alkyl group of 4 to 20 carbon atoms which may have an oxygen atom (═O) as a substituent; and q represents 0 or 1.

Hereinbelow, the anion moieties represented by general formulas (1) to (4) will be described in this order as anion moieties (1) to (4).

{Anion Moiety (1)}

In general formula (1), X⁰ represents a hydrocarbon group of 1 to 30 carbon atoms which may have a substituent.

The hydrocarbon group for X⁰ may be either an aromatic hydrocarbon group or an aliphatic hydrocarbon group.

The aromatic hydrocarbon group is a hydrocarbon group having an aromatic ring. The aromatic hydrocarbon group preferably has 3 to 30 carbon atoms, more preferably 5 to 30, still more preferably 5 to 20, still more preferably 6 to 15, and most preferably 6 to 12. Here, the number of carbon atoms within a substituent(s) is not included in the number of carbon atoms of the aromatic hydrocarbon group.

Specific examples of aromatic hydrocarbon groups include an aryl group which is an aromatic hydrocarbon ring having one hydrogen atom removed therefrom, such as a phenyl group, a biphenyl group, a fluorenyl group, a naphthyl group, an anthryl group or a phenanthryl group; and an alkylaryl group such as a benzyl group, a phenethyl group, a 1-naphthylmethyl group, a 2-naphthylmethyl group, a 1-naphthylethyl group, or a 2-naphthylethyl group. The alkyl chain within the arylalkyl group preferably has 1 to 4 carbon atom, more preferably 1 or 2, and most preferably 1.

The aromatic hydrocarbon group may have a substituent. For example, part of the carbon atoms constituting the aromatic ring within the aromatic hydrocarbon group may be substituted with a hetero atom, or a hydrogen atom bonded to the aromatic ring within the aromatic hydrocarbon group may be substituted with a substituent.

In the former example, a heteroaryl group in which part of the carbon atoms constituting the ring within the aforementioned aryl group has been substituted with a hetero atom such as an oxygen atom, a sulfur atom or a nitrogen atom, and a heteroarylalkyl group in which part of the carbon atoms constituting the aromatic hydrocarbon ring within the aforementioned arylalkyl group has been substituted with the aforementioned heteroatom can be used. In such a case, with respect to the partial structure “X⁰-Q¹-” of the anion moiety in general formula (1), the atom within X⁰ to which Q¹ is bonded is preferably a carbon atom.

In the latter example, as the substituent for the aromatic hydrocarbon group, an alkyl group, an alkoxy group, a halogen atom, a halogenated alkyl group, a hydroxyl group, an oxygen atom (═O), a substituent containing a nitrogen atom (nitrogen atom-containing substituent described later) or the like can be used.

The alkyl group as the substituent for the aromatic hydrocarbon group is preferably an alkyl group of 1 to 5 carbon atoms, and a methyl group, an ethyl group, a propyl group, an n-butyl group or a tert-butyl group is particularly desirable.

The alkoxy group as the substituent for the aromatic hydrocarbon group is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the halogen atom as the substituent for the aromatic hydrocarbon group include a fluorine atom, a chlorine atom, a bromine atom and an iodine atom, and a fluorine atom is preferable.

Example of the halogenated alkyl group as the substituent for the aromatic hydrocarbon group includes a group in which part or all of the hydrogen atoms within the aforementioned alkyl group have been substituted with the aforementioned halogen atoms.

The aliphatic hydrocarbon group for X⁰ may be either a saturated aliphatic hydrocarbon group, or an unsaturated aliphatic hydrocarbon group. Further, the aliphatic hydrocarbon group may be linear, branched or cyclic, or a combination thereof.

In the aliphatic hydrocarbon group for X⁰, part of the carbon atoms constituting the aliphatic hydrocarbon group may be substituted with a substituent group containing a hetero atom, or part or all of the hydrogen atoms constituting the aliphatic hydrocarbon group may be substituted with a substituent group containing a hetero atom.

As the “hetero atom” for X⁰, there is no particular limitation as long as it is an atom other than carbon and hydrogen.

Examples of the halogen atom include a fluorine atom, a chlorine atom, an iodine atom and a bromine atom.

The substituent group containing a hetero atom may consist of a hetero atom, or may be a group containing a group or atom other than a hetero atom.

Specific examples of the substituent group for substituting part of the carbon atoms include —O—, —C(═O)—O—, —C(═O)—, —O—C(═O)—O—, —C(═O)—NH—, —NH— (the H may be replaced with a substituent such as an alkyl group or an acyl group), —S—, —S(═O)₂— and —S(═O)₂—O—. When the aliphatic hydrocarbon group is cyclic, the aliphatic hydrocarbon group may contain any of these substituent groups in the ring structure.

Examples of the substituent group for substituting part or all of the hydrogen atoms include an alkoxy group, a halogen atom, a halogenated alkyl group, a hydroxyl group, an oxygen atom (═O) and a substituent containing a nitrogen atom described later (e.g., a cyano group).

The aforementioned alkoxy group is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the aforementioned halogen atom include a fluorine atom, a chlorine atom, a bromine atom and an iodine atom, and a fluorine atom is preferable.

Example of the aforementioned halogenated alkyl group includes a group in which part or all of the hydrogen atoms within an alkyl group of 1 to 5 carbon atoms (e.g., a methyl group, an ethyl group, a propyl group, an n-butyl group or a tert-butyl group) have been substituted with the aforementioned halogen atoms.

As the aliphatic hydrocarbon group, a linear or branched saturated hydrocarbon group, a linear or branched monovalent unsaturated hydrocarbon group, or a cyclic aliphatic hydrocarbon group (aliphatic cyclic group) is preferable. Further, a group in which a linear or branched, saturated or unsaturated hydrocarbon group is bonded to an aliphatic cyclic group is also preferable.

The linear saturated hydrocarbon group (alkyl group) preferably has 1 to 20 carbon atoms, more preferably 1 to 15, and most preferably 1 to 10. Specific examples include a methyl group, an ethyl group, a propyl group, a butyl group, a pentyl group, a hexyl group, a heptyl group, an octyl group, a nonyl group, a decyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a henicosyl group and a docosyl group.

The branched saturated hydrocarbon group (alkyl group) preferably has 3 to 20 carbon atoms, more preferably 3 to 15, and most preferably 3 to 10. Specific examples include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group and a 4-methylpentyl group.

The unsaturated hydrocarbon group preferably has 3 to 10 carbon atoms, more preferably 3 to 5, still more preferably 3 or 4, and most preferably 3. Examples of linear monovalent unsaturated hydrocarbon groups include a propenyl group (an allyl group) and a butynyl group. Examples of branched monovalent unsaturated hydrocarbon groups include a 1-methylpropenyl group and a 2-methylpropenyl group.

Among the above-mentioned examples, as the unsaturated hydrocarbon group, a propenyl group is particularly desirable.

The aliphatic cyclic group may be either a monocyclic group or a polycyclic group. The aliphatic cyclic group preferably has 3 to 30 carbon atoms, more preferably 5 to 30, still more preferably 5 to 20, still more preferably 6 to 15, and most preferably 6 to 12.

As the aliphatic cyclic group, a group in which one or more hydrogen atoms have been removed from a monocycloalkane or a polycycloalkane such as a bicycloalkane, tricycloalkane or tetracycloalkane can be used. Specific examples include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane.

When the aliphatic cyclic group contains a hetero atom-containing substituent group in the ring structure thereof, the hetero atom-containing substituent group is preferably —O—, —C(═O)—O—, —S—, —S(═O)₂— or —S(═O)₂—O—.

Examples of the aliphatic cyclic group include a lactone-containing cyclic group and an —SO₂— containing cyclic group.

The term “lactone-containing cyclic group” refers to a cyclic group including a ring containing a —O—C(O)— structure (lactone ring). The term “lactone ring” refers to a single ring containing a —O—C(O)— structure, and this ring is counted as the first ring. A lactone-containing cyclic group in which the only ring structure is the lactone ring is referred to as a monocyclic group, and groups containing other ring structures are described as polycyclic groups regardless of the structure of the other rings.

The lactone-containing cyclic group may be either a monocyclic group or a polycyclic group.

As the lactone-containing cyclic group, there is no particular limitation, and an arbitrary group may be used. Specific examples of lactone-containing monocyclic groups include a group in which one hydrogen atom has been removed from a 4- to 6-membered lactone ring, such as a group in which one hydrogen atom has been removed from β-propionolactone, a group in which one hydrogen atom has been removed from γ-butyrolactone, and a group in which one hydrogen atom has been removed from δ-valerolactone. Further, specific examples of lactone-containing polycyclic groups include groups in which one hydrogen atom has been removed from a lactone ring-containing bicycloalkane, tricycloalkane or tetracycloalkane.

An “—SO₂— containing cyclic group” refers to a cyclic group having a ring containing —SO₂— within the ring structure thereof, i.e., a cyclic group in which the sulfur atom (S) within —SO₂— forms part of the ring skeleton of the cyclic group. The ring containing —SO₂— within the ring skeleton thereof is counted as the first ring. A cyclic group in which the only ring structure is the ring that contains —SO₂— in the ring skeleton thereof is referred to as a monocyclic group, and a group containing other ring structures is described as a polycyclic group regardless of the structure of the other rings. The —SO₂— containing cyclic group may be either a monocyclic group or a polycyclic group.

As the —SO₂— containing cyclic group, a cyclic group containing —O—SO₂— within the ring skeleton thereof, i.e., a cyclic group containing a sultone ring in which —O—S— within the —O—SO₂— group forms part of the ring skeleton thereof is particularly desirable.

The —SO₂— containing cyclic group preferably has 3 to 30 carbon atoms, more preferably 4 to 20, still more preferably 4 to 15, and most preferably 4 to 12. Herein, the number of carbon atoms refers to the number of carbon atoms constituting the ring skeleton, excluding the number of carbon atoms within a substituent.

Examples of the —SO₂— containing aliphatic cyclic group include aliphatic cyclic groups in which part of the carbon atoms constituting the ring skeleton has been substituted with a —SO₂— group or a —O—SO₂— group and has at least one hydrogen atom removed from the aliphatic hydrocarbon ring. Specific examples include an aliphatic hydrocarbon ring in which a —CH₂— group constituting the ring skeleton thereof has been substituted with a —SO₂— group and has at least one hydrogen atom removed therefrom; and an aliphatic hydrocarbon ring in which a —CH₂—CH₂— group constituting the ring skeleton has been substituted with a —O—SO₂— group and has at least one hydrogen atom removed therefrom.

The alicyclic hydrocarbon group preferably has 3 to 20 carbon atoms, and more preferably 3 to 12 carbon atoms.

The alicyclic hydrocarbon group may be either a monocyclic group or a polycyclic group. As the monocyclic group, a group in which two hydrogen atoms have been removed from a monocycloalkane of 3 to 6 carbon atoms is preferable. Examples of the monocycloalkane include cyclopentane and cyclohexane. As the polycyclic group, a group in which two hydrogen atoms have been removed from a polycycloalkane of 7 to 12 carbon atoms is preferable. Examples of the polycycloalkane include adamantane, norbornane, isobornane, tricyclodecane and tetracyclododecane.

The —SO₂— containing cyclic group may have a substituent. Examples of the substituent include an alkyl group, an alkoxy group, a halogen atom, a halogenated alkyl group, a hydroxy group, an oxygen atom (═O), —COOR″, —OC(═O)R″, a hydroxyalkyl group and a cyano group.

The alkyl group for the substituent is preferably an alkyl group of 1 to 6 carbon atoms. Further, the alkyl group is preferably a linear alkyl group or a branched alkyl group. Specific examples include a methyl group, an ethyl group, a propyl group, an isopropyl group, an n-butyl group, an isobutyl group, a tert-butyl group, a pentyl group, an isopentyl group, a neopentyl group and a hexyl group. Among these, a methyl group or ethyl group is preferable, and a methyl group is particularly desirable.

As the alkoxy group for the substituent, an alkoxy group of 1 to 6 carbon atoms is preferable. Further, the alkoxy group is preferably a linear alkoxy group or a branched alkyl group. Specific examples of the alkoxy group include the aforementioned alkyl groups for the substituent having an oxygen atom (—O—) bonded thereto.

Examples of the halogen atom for the substituent include a fluorine atom, a chlorine atom, a bromine atom and an iodine atom, and a fluorine atom is preferable.

Examples of the halogenated alkyl group for the substituent include groups in which part or all of the hydrogen atoms within the aforementioned alkyl groups has been substituted with the aforementioned halogen atoms.

As examples of the halogenated lower alkyl group for the substituent, groups in which part or all of the hydrogen atoms of the aforementioned alkyl groups for the substituent have been substituted with the aforementioned halogen atoms can be given. As the halogenated alkyl group, a fluorinated alkyl group is preferable, and a perfluoroalkyl group is particularly desirable.

In the —COOR″ group and the —OC(═O)R″ group, R″ represents a hydrogen atom or a linear, branched or cyclic alkyl group of 1 to 15 carbon atoms.

When R″ represents a linear or branched alkyl group, it is preferably an alkyl group of 1 to 10 carbon atoms, more preferably an alkyl group of 1 to 5 carbon atoms, and most preferably a methyl group or an ethyl group.

When R″ is a cyclic alkyl group (cycloalkyl group), it preferably has 3 to 15 carbon atoms, more preferably 4 to 12 carbon atoms, and most preferably 5 to 10 carbon atoms. As examples of the cycloalkyl group, groups in which one or more hydrogen atoms have been removed from a monocycloalkane or a polycycloalkane such as a bicycloalkane, tricycloalkane or tetracycloalkane, which may or may not be substituted with a fluorine atom or a fluorinated alkyl group, may be used. Specific examples include groups in which one or more hydrogen atoms have been removed from a monocycloalkane such as cyclopentane and cyclohexane; and groups in which one or more hydrogen atoms have been removed from a polycycloalkane such as adamantane, norbornane, isobornane, tricyclodecane or tetracyclododecane.

The hydroxyalkyl group for the substituent preferably has 1 to 6 carbon atoms, and specific examples thereof include the aforementioned alkyl groups for the substituent in which at least one hydrogen atom has been substituted with a hydroxy group.

Specific examples of the lactone-containing cyclic group include groups represented by formulas (L1) to (L6) shown below, and specific examples of the —SO₂-containing cyclic group include groups represented by formulas (S1) to (S4) shown below.

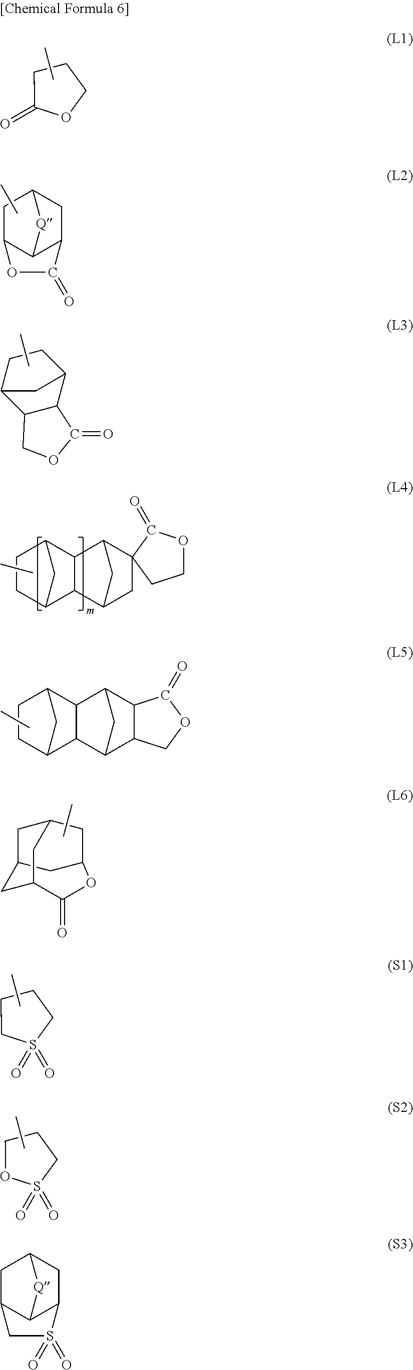

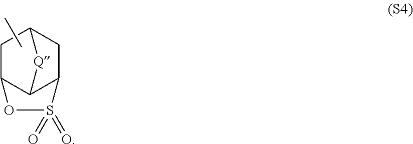

In the formulas, Q″ represents an alkylene group of 1 to 5 carbon atoms, —O—, —S—, —O—R⁹⁸— or —S—R⁹⁹— (wherein each of R⁹⁸ and R⁹⁹ independently represents an alkylene group of 1 to 5 carbon atoms); and m represents 0 or 1.

Specific examples of the alkylene group for Q″, R⁹⁸ and R⁹⁹ include a methylene group [—CH₂—]; alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; an ethylene group [—CH₂CH₂—]; alkylethylene groups such as —CH(CH₃)CH₂—, —CH(CH₃)CH(CH₃)—, —C(CH₃)₂CH₂—, —CH(CH₂CH₃)CH₂—, and —CH(CH₂CH₃)CH₂—; a trimethylene group (n-propylene group) [—CH₂CH₂CH₂—]; alkyltrimethylene groups such as —CH(CH₃)CH₂CH₂—, and —CH₂CH(CH₃)CH₂—; a tetramethylene group [—CH₂CH₂CH₂CH₂—]; alkyltetramethylene groups such as —CH(CH₃)CH₂CH₂CH₂—, and —CH₂CH(CH₃)CH₂CH₂—; and a pentamethylene group [—CH₂CH₂CH₂CH₂CH₂—].

In these aliphatic cyclic groups, part of the hydrogen atoms bonded to the carbon atoms constituting the ring structure may be substituted with a substituent. Examples of substituents include an alkyl group, an alkoxy group, a halogen atom, a halogenated alkyl group, a hydroxyl group and an oxygen atom (═O).

As the alkyl group, an alkyl group of 1 to 5 carbon atoms is preferable, and a methyl group, an ethyl group, a propyl group, an n-butyl group or a tert-butyl group is particularly desirable.

As the alkoxy group and the halogen atom, the same groups as the aforementioned substituent groups for substituting a part or all of the hydrogen atoms can be used.

As a group in which a linear or branched, saturated or unsaturated hydrocarbon group is bonded to an aliphatic cyclic group, for example, a group in which a linear or branched, saturated hydrocarbon group is bonded to a carbon atom constituting the ring structure of an aliphatic cyclic group is preferable, a group in which a linear, saturated hydrocarbon group is bonded to the carbon atom is more preferable, and a group in which a linear alkylene group is bonded to the carbon atom is particularly desirable.

Further, especially when X⁰ represents a group containing a nitrogen atom, examples of X⁰ include a hydrocarbon group having a substituent containing a nitrogen atom (hereafter, this group is referred to as “nitrogen-containing substituent”) and a heterocyclic group containing a nitrogen atom as the hetero atom (hereafter, this group is referred to as “nitrogen-containing heterocyclic group”). These organic groups may have, apart from the nitrogen-containing substituent, a substituent other than a nitrogen-containing substituent (hereafter, referred to as “non-nitrogen-containing substituent”).

As the nitrogen-containing heterocyclic group for X⁰, a monovalent group in which one hydrogen atom has been removed from a heterocyclic group containing a nitrogen atom as the hetero atom (i.e., a nitrogen-containing heterocyclic group) can be mentioned. Examples of the nitrogen-containing heterocyclic group include unsaturated, monocyclic nitrogen-containing hetero rings, such as pyridine, pyrrole, pyrrazole, imidazole, 1,2,3-triazole, 1,2,4-triazole, tetrazole, pyrimidine, pyrazine and 1,3,5-triazine; saturated, monocyclic nitrogen-containing hetero rings, such as piperidine, piperazine and pyrrolidine; and polycyclic nitrogen-containing hetero rings, such as quinoline, isoquinoline, indole, pyrrolo[2,3-b]pyridine, indazole, benzimidazole, benzotriazole, carbazole, acridine, 1,5-diazabicyclo[4.3.0]-5-nonene, 1,8-diazabicyclo[5.4.0]-7-undecene, hexamethylenetetramine and 1,4-diazabicyclo[2.2.2]octane.

The nitrogen-containing heterocyclic group may be either a monocyclic group or a polycyclic group. The nitrogen-containing heterocyclic group has 3 to 30 carbon atoms, more preferably 5 to 30 carbon atoms, and still more preferably 5 to 20 carbon atoms.

Examples of the nitrogen-containing substituent for X⁰ include the aforementioned nitrogen-containing heterocyclic group, as well as an amino group (H₂N—), an imino group (HN═), a cyano group (N≡C—) and an ammonio group (⁺NH₃—). These nitrogen-containing substituents may have part or all of the hydrogen atoms substituted with a non-nitrogen-containing substituent.

Specific examples of the nitrogen-containing substituent for X⁰ include nitrogen-containing heterocyclic groups, such as a 2-pyridyl group, a 3-pyridyl group, a 4-pyridyl group and a piperidino group; an amino group; an alkylamino group; a dialkylamino group; an imino group; an alkylimino group; a cyano group; and a trialkylammonio group. Among these, a nitrogen-containing heterocyclic group such as a 4-pyridyl group is preferable.

Examples of the non-nitrogen-containing substituent for X⁰ include an alkyl group, an aryl group, an alkoxy group, a halogen atom, a halogenated alkyl group, a hydroxyl group and an oxygen atom (═O).

As the alkyl group, an alkyl group of 1 to 5 carbon atoms is preferable, and a methyl group, an ethyl group, a propyl group, an n-butyl group or a tert-butyl group is particularly desirable.

Examples of aryl groups include a phenyl group, a tolyl group and a naphthyl group.

Examples of halogen atoms include a fluorine atom, a chlorine atom, an iodine atom and a bromine atom.

The aforementioned alkoxy group is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the aforementioned halogen atom include a fluorine atom, a chlorine atom, a bromine atom and an iodine atom, and a fluorine atom is preferable.

Example of the aforementioned halogenated alkyl group includes a group in which part or all of the hydrogen atoms within an alkyl group of 1 to 5 carbon atoms (e.g., a methyl group, an ethyl group, a propyl group, an n-butyl group or a tert-butyl group) have been substituted with the aforementioned halogen atoms.

When X⁰ represents a hydrocarbon group which has a nitrogen-containing substituent, the hydrocarbon group may be either an aliphatic hydrocarbon group or an aromatic hydrocarbon group, and examples thereof include the same aliphatic hydrocarbon groups and aromatic hydrocarbon groups as those described above for the “hydrocarbon group for X⁰”.

Specific examples of the group represented by X⁰ that contains a nitrogen atom include nitrogen-containing heterocyclic groups, such as a 2-pyridyl group, a 3-pyridyl group and a 4-pyridyl group; aminoalkyl groups, such as an aminomethyl group, a 1-aminoethyl group and a 2-aminoethyl group; alkylaminoalkyl groups, such as a methylaminomethyl group; dialkylaminoalkyl groups, such as a dimethylaminomethyl group; aminoaryl groups, such as a 2-aminophenyl group and a 4-aminophenyl group; alkylaminoaryl groups, such as a (methylamino)phenyl group; and dialkylaminoaryl groups, such as a (dimethylamino)phenyl group and a (diethylamino)phenyl group. Among these, a nitrogen-containing heterocyclic group such as a 4-pyridyl group is preferable.

In the present invention, as X⁰, a cyclic group which may have a substituent is preferable. The cyclic group may be either an aromatic hydrocarbon group which may have a substituent, or an aliphatic cyclic group which may have a substituent.

As the aromatic hydrocarbon group, a naphthyl group which may have a substituent, or a phenyl group which may have a substituent is preferable.

As the aliphatic cyclic group which may have a substituent, an aliphatic polycyclic group which may have a substituent is preferable. As the aliphatic polycyclic group, the aforementioned group in which one or more hydrogen atoms have been removed from a polycycloalkane, and groups represented by the aforementioned formulas (L2) to (L6), (S3) and (S4) are preferable.

In general formula (1), Q¹ represents a single bond or a divalent linking group containing a carbonyl group.

Q¹ may contain an atom other than carbon and oxygen. Examples of atoms other than carbon and oxygen include a hydrogen atom, a sulfur atom and a nitrogen atom.

Examples of divalent linking groups that contain a carbonyl group include non-hydrocarbon, carbonyl group-containing linking groups, such as an ester bond (—C(═O)—O—), an amide bond (—C(═O)—NH—), a carbonyl group (—C(═O)—), a carbonate bond (—O—C(═O)—O—); and groups that contain a non-hydrocarbon, carbonyl group-containing linking group.

Examples of groups that contain a non-hydrocarbon, carbonyl group-containing linking group include combinations of any of such non-hydrocarbon, carbonyl group-containing linking groups with a groups selected from an alkylene group, an oxygen atom (an ether bond: —O—) and a sulfonyl group.

Specific examples of such combinations include —O—R⁹¹—O—C(═O)—, —C(═O)—O—R⁹²—, —C(═O)—O—R⁹³—O—C(═O)—, —R⁹⁴—C(═O)—O—R⁹⁵—O—C(═O)—, —S(═O)₂—O—R⁹⁶—O—C(═O)— (in the formulas, each of R⁹¹ to R⁹⁶ independently represents an alkylene group).

The alkylene group for R⁹¹ to R⁹⁶ is preferably a linear or branched alkylene group, and preferably has 1 to 12 carbon atoms, more preferably 1 to 5, and most preferably 1 to 3.

Specific examples of alkylene groups include a methylene group [—CH₂—]; alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; an ethylene group [—CH₂CH₂—]; alkylethylene groups such as —CH(CH₃)CH₂—, —CH(CH₃)CH(CH₃)—, —C(CH₃)₂CH₂— and —CH(CH₂CH₃)CH₂—, and —CH(CH₂CH₃)CH₂—; a trimethylene group (n-propylene group) [—CH₂CH₂CH₂—]; alkyltrimethylene groups such as —CH(CH₃)CH₂CH₂— and —CH₂CH(CH₃)CH₂—; a tetramethylene group [—CH₂CH₂CH₂CH₂—]; alkyltetramethylene groups such as —CH(CH₃)CH₂CH₂CH₂— and —CH₂CH(CH₃)CH₂CH₂—; and a pentamethylene group [—CH₂CH₂CH₂CH₂CH₂—].

In general formula (1), p represents an integer of 0 to 3, preferably 0 to 2, and most preferably 0 or 1.

More specifically, preferable examples of the anion moiety (1) include

an anion moiety represented by general formula (1-1-0) shown below,

an anion moiety represented by general formula (1-1-1) shown below,

an anion moiety represented by general formula (1-1-2) shown below,

an anion moiety represented by general formula (1-1-3) shown below,

an anion moiety represented by general formula (1-1-4) shown below,

an anion moiety represented by general formula (1-1-5) shown below, and

an anion moiety represented by general formula (1-1-6) shown below.

Anion Moiety Represented by General Formula (1-1-0)

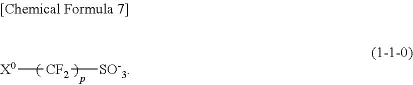

In general formula (1-1-0), X⁰ represents a hydrocarbon group of 1 to 30 carbon atoms which may have a substituent; and p represents an integer of 0 to 3.

In formula (1-1-0), X⁰ and p are respectfully the same as defined for X⁰ and p in general formula (1).

As X⁰, a linear aliphatic hydrocarbon group which may have a substituent is preferable. Among these, a halogenated alkyl group in which part or all of the hydrogen atoms of the aforementioned aliphatic hydrocarbon group has been substituted with a halogen atom is preferable.

p is preferably 0 to 3.

Anion Moiety Represented by General Formula (1-1-1)

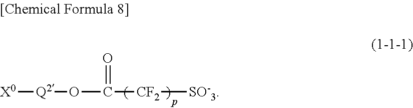

In formula (1-1-1), Q²′ represents a single bond or an alkylene group; and X⁰ and p are the same as defined above.

In formula (1-1-1), as X⁰, an aliphatic cyclic group which may have a substituent, a linear aliphatic hydrocarbon group which may have a substituent, or an aromatic hydrocarbon group which may have a substituent is preferable. Of these, an aliphatic cyclic group which contains a hetero atom-containing substituent in the ring structure thereof is more preferable

As the alkylene group for Q²′, the same alkylene groups as those described above for Q¹ can be mentioned.

As Q²′, a single bond or a methylene group is particularly desirable. Especially, when X⁰ is an aliphatic cyclic group which may have a substituent, Q²′ is preferably a single bond. On the other hand, when X⁰ is an aromatic hydrocarbon group, Q²′ is preferably a methylene group.

p is preferably 1 or 2, and most preferably 1.

Specific examples of preferable anions represented by general, formula (1-1-1) are shown below.

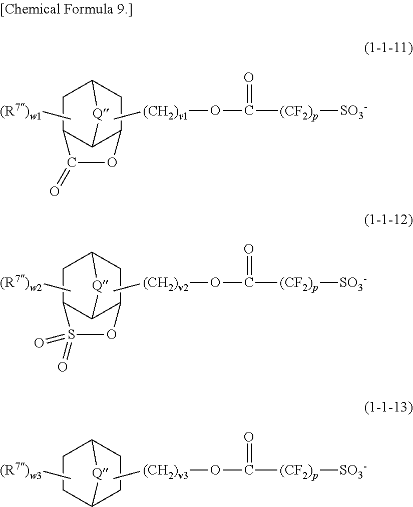

In the formulas, Q″ is the same as defined above; R⁷″ represents a substituent; each of w1 to w3 independently represents an integer of 0 to 3; each of v1 to v3 independently represents an integer of 0 to 5; and p represents an integer of 1 to 3.

In the formulas, as the substituent for R⁷″, the same groups as those which the aforementioned aliphatic hydrocarbon group or aromatic hydrocarbon group for X⁰ may have as a substituent can be mentioned.

If there are two or more of the R⁷″ group, as indicated by the values w1 to w3, then the two or more of the R⁷″ groups may be the same or different from each other.

It is preferable that each of v1 to v3 independently represents an integer of 0 to 3, most preferably 0.

It is preferable that each of w1 to w3 independently represents an integer of 0 to 2, and most preferably 0.

p is preferably 1 or 2, and most preferably 1.

Anion Moiety Represented by General Formula (1-1-2)

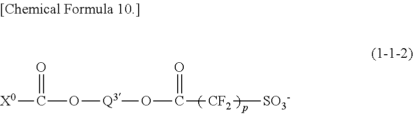

In formula (1-1-2), X⁰ and p are the same as defined above; and Q³′ represents an alkylene group.

In formula (1-1-2), as X⁰, an aliphatic cyclic group which may have a substituent, a linear aliphatic hydrocarbon group which may have a substituent, or an aromatic hydrocarbon group which may have a substituent is preferable.

p is preferably 1 or 2, and most preferably 1.

As the alkylene group for Q³′, the same alkylene groups as those described above for Q¹ can be mentioned.

Specific examples of preferable anions represented by general formula (1-1-2) are shown below.

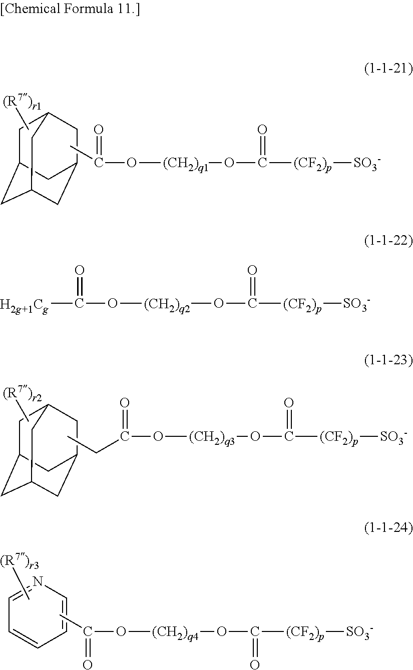

In the formulas, p is the same as defined above; R⁷″ represents a substituent; each of r1 to r3 independently represents an integer of 0 to 3; each of q1 to q4 independently represents an integer of 1 to 12; and g represents an integer of 1 to 20.

In the formulas, as the substituent for R⁷″, the same groups as those described above for R⁷″ can be mentioned.

If there are two or more of the R⁷″ group, as indicated by the values r1 to r3, then the two or more of the R⁷″ groups may be the same or different from each other.

It is preferable that each of r1 to r3 independently represent an integer of 0 to 2, more preferably 0 or 1, and still more preferably 0.

It is preferable that each of q1 to q4 independently represent 1 to 8, more preferably 1 to 5, and still more preferably 1 to 3.

g is preferably 1 to 15, and more preferably 1 to 10.

p is preferably 1 or 2, and most preferably 1.

Anion Moiety Represented by General Formula (1-1-3)

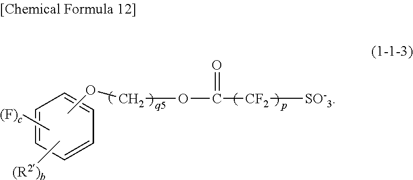

In the formula, p is the same as defined above; q5 represents an integer of 0 to 5; R²′ represents an alkyl group, an alkoxy group, a halogen atom (excluding fluorine), a halogenated alkyl group, a hydroxy group, an oxygen atom (═O), —COOR″, —OC(═O)R″, a hydroxyalkyl group or a cyano group; b represents an integer of 0 to 2, and c represents an integer of 1 to 5, provided that 1≦b+c≦5.

p is preferably 1 or 2, and most preferably 1.

q5 is preferably 1 to 4, more preferably 1 or 2, and most preferably 2.

Examples of the alkyl group, alkoxy group, halogen atom (excluding fluorine) and halogenated alkyl group for R²′ include the same groups as those described above for the substituent which a cyclic group represented by X may have.

With respect to —COOR″ and —OC(═O)R″ for R²′, R″ is the same as defined for R″ in the aforementioned structural unit (a2).

As the hydroxyalkyl group for R²′, groups in which at least one hydrogen atom of the aforementioned alkyl groups for R² has been substituted with a hydroxy group can be mentioned.

b is most preferably 0.

c is preferably 2 to 5, and most preferably 5.

However, 1≦b+c≦5.

Anion Moiety Represented by General Formula (1-1-4)

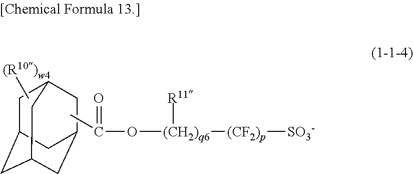

In the formula, p is the same as defined above; q6 represents an integer of 1 to 12; w4 represents an integer of 0 to 3; R¹⁰″ represents a substituent; R¹⁰″ represents a hydrogen atom, an alkyl group of 1 to 5 carbon atoms or a fluorinated alkyl group of 1 to 5 carbon atoms; and R¹¹″ represents a hydrogen atom, an alkyl group of 1 to 5 carbon atoms or a halogenated alkyl group of 1 to 5 carbon atoms.

Examples of the substituent for R¹⁰″ include an alkyl group, an alkoxy group, a halogen atom, a halogenated alkyl group, a hydroxyl group, an oxygen atom (═O) and a cyano group.

As the alkyl group, an alkyl group of 1 to 5 carbon atoms is preferable, and a methyl group, an ethyl group, a propyl group, an n-butyl group or a tert-butyl group is particularly desirable.

The aforementioned alkoxy group is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the aforementioned halogen atom include a fluorine atom, a chlorine atom, a bromine atom and an iodine atom, and a fluorine atom is preferable.

Example of the aforementioned halogenated alkyl group includes a group in which part or all of the hydrogen atoms within an alkyl group of 1 to 5 carbon atoms (e.g., a methyl group, an ethyl group, a propyl group, an n-butyl group or a tert-butyl group) have been substituted with the aforementioned halogen atoms.

If there are two or more of the R¹⁰″ group, as indicated by the value w4, then the two or more of the R¹⁰″ groups may be the same or different from each other.

p is preferably 1 or 2, and most preferably 1.

q6 is preferably 1 to 5, more preferably 1 to 3, and most preferably 1.

w4 is preferably an integer of 0 to 2, more preferably 0 or 1, and still more preferably 0.

R¹¹″ represents a hydrogen atom, an alkyl group of 1 to 5 carbon atoms or a halogenated alkyl group of 1 to 5 carbon atoms. As the alkyl group and the halogenated alkyl group, the same alkyl groups and halogenated alkyl groups as those described above for R¹⁰″ can be mentioned.

Anion Moiety Represented by General Formula (1-1-5)

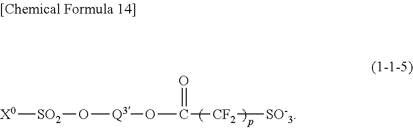

In formula (1-1-5), X⁰, Q³′ and p are the same as defined above.

In general formula (1-1-5), as X⁰, an aliphatic cyclic group which may have a substituent is preferable.

p is preferably 1 or 2, and most preferably 1.

Specific examples of preferable anions represented by general formula (1-1-5) are shown below.

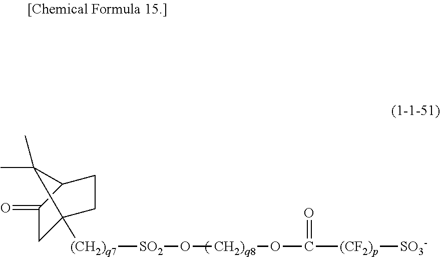

In the formula, p is the same as defined above; and each of q7 and q8 independently represents an integer of 1 to 12.

Anion Moiety Represented by General Formula (1-1-6)

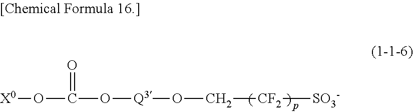

In formula (1-1-6), X⁰, Q³′ and p are the same as defined above.

In general formula (1-1-6), as X⁰, an aliphatic cyclic group which may have a substituent is preferable.

p is preferably 1 or 2, and most preferably 1.

Specific examples of preferable anions represented by general formula (1-1-6) are shown below.

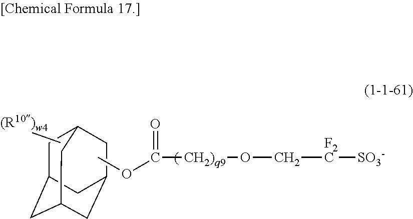

In the formula, R¹⁰″, w4 and p are the same as defined above; and q9 represents an integer of 1 to 12.

w4 is preferably an integer of 0 to 2, and more preferably 0 or 1.

q9 is preferably 1 to 5, more preferably 1 to 3, and most preferably 1.

In general formula (1-1-61), by directly bonding the terminal oxygen atom of —C(═O)—O— to a tertiary carbon atom of R¹⁰″, the R¹⁰″ group can form a tertiary alkyl ester-type acid dissociable, dissolution inhibiting group described later.

{Anion Moiety (2)}

In general formula (2), X⁰ represents a hydrocarbon group of 3 to 30 carbon atoms which may have a substituent.

The hydrocarbon group represented by X⁰ is the same as defined for the hydrocarbon group represented by X⁰ in general formula (1).

In general formula (2), Q² represents a single bond or an alkylene group.

The alkylene group for Q² is preferably a linear or branched alkylene group, and preferably has 1 to 12 carbon atoms, more preferably 1 to 5, and most preferably 1 to 3.

Specific examples of alkylene groups include a methylene group [—CH₂—]; alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; an ethylene group [—CH₂CH₂—]; alkylethylene groups such as —CH(CH₃)CH₂—, —CH(CH₃)CH(CH₃)—, —C(CH₃)₂CH₂— and —CH(CH₂CH₃)CH₂—, and —CH(CH₂CH 3)CH₂—; a trimethylene group (n-propylene group) [—CH₂CH₂CH₂—]; alkyltrimethylene groups such as —CH(CH₃)CH₂CH₂— and —CH₂CH(CH₃)CH₂—; a tetramethylene group [—CH₂CH₂CH₂CH₂—]; alkyltetramethylene groups such as —CH(CH₃)CH₂CH₂CH₂— and —CH₂CH(CH₃)CH₂CH₂—; and a pentamethylene group [—CH₂CH₂CH₂CH₂CH₂—].

In general formula (2), p represents an integer of 1 to 3, preferably 1 or 2, and most preferably 2.

Specific examples of anion moieties preferable as the anion moiety (2) are shown below.

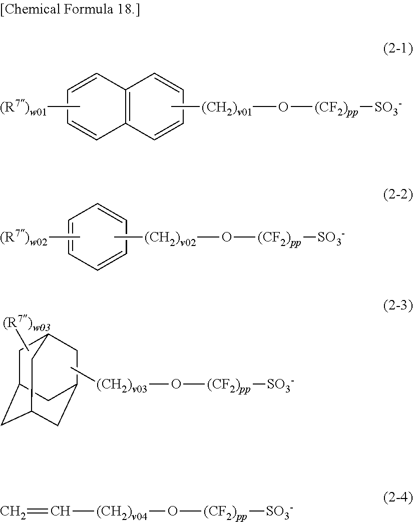

In the formulas, R⁷″ represents a substituent; each of w01 to w03 independently represents an integer of 0 to 3; each of v01 to v04 independently represents an integer of 0 to 5; and pp represents an integer of 1 to 3.

In the formulas above, R⁷″ is the same as defined above.

If there are two or more of the R⁷″ group, as indicated by the values w01 to w03, then the two or more of the R⁷″ groups may be the same or different from each other.

It is preferable that each of v01 to v04 independently represents an integer of 0 to 3, and preferably 0 or 1.

It is preferable that each of w01 to w03 independently represents an integer of 0 to 2, and most preferably 0.

pp is preferably 1 or 2, and most preferably 2.

{Anion Moiety (3)}

In general formula (3), X¹⁰ represents a hydrocarbon group of 1 to 30 carbon atoms which may have a substituent.

The hydrocarbon group for X¹⁰ may be either an aromatic hydrocarbon group or an aliphatic hydrocarbon group.

The aromatic hydrocarbon group for X¹⁰ is the same as defined for the aromatic hydrocarbon group for X⁰ in general formula (1).

The aliphatic hydrocarbon group for X¹⁰ is the same as defined for the aliphatic hydrocarbon group for X⁰ in general formula (1). As the aliphatic hydrocarbon group, a linear or branched saturated hydrocarbon group, a linear or branched monovalent unsaturated hydrocarbon group, or a cyclic aliphatic hydrocarbon group (aliphatic cyclic group) is preferable. Further, a group in which a linear or branched, saturated or unsaturated hydrocarbon group is bonded to an aliphatic cyclic group is also preferable.

The linear saturated hydrocarbon group (alkyl group) preferably has 1 to 20 carbon atoms, more preferably 1 to 15, and most preferably 3 to 12. Specific examples of the linear saturated hydrocarbon group include the same linear saturated hydrocarbon groups as those described above for X⁰ in general formula (1).

Examples of the branched saturated hydrocarbon group (alkyl group) include the same branched, saturated hydrocarbon groups as those described above for X⁰ in general formula (1).

The unsaturated hydrocarbon group preferably has 2 to 10 carbon atoms, more preferably 2 to 5, still more preferably 2 to 4, and most preferably 3. Specific examples thereof include the same unsaturated hydrocarbon groups as those described above for X⁰ in general formula (1).

Examples of the group in which a linear or branched, saturated or unsaturated hydrocarbon group is bonded to an aliphatic cyclic group include the same groups as those described above for X⁰ in general formula (1).

In the present invention, as X¹⁰, a cyclic group which may have a substituent is preferable.

The cyclic group may be either an aromatic hydrocarbon group which may have a substituent, or an aliphatic cyclic group which may have a substituent.

As the aromatic hydrocarbon group, a naphthyl group which may have a substituent, or a phenyl group which may have a substituent is preferable.

As the aliphatic cyclic group which may have a substituent, an aliphatic polycyclic group which may have a substituent is preferable. As the aliphatic polycyclic group, the aforementioned group in which one or more hydrogen atoms have been removed from a polycycloalkane, and groups represented by the aforementioned formulas (L2) to (L6), (S3) and (S4) are preferable.

In general formula (3), Q³ represents a single bond or a divalent linking group.

Examples of the divalent linking group for Q³ include the following:

an alkylene group or a fluorinated alkylene group;

non-hydrocarbon, oxygen-containing linking groups, such as an oxygen atom (ether bond: —O—), an ester bond (—C(═O)—O—), an amide bond (—C(═O)—NH—), a carbonyl group (—C(═O)—) and a carbonate bond (—O—C(═O)—O—); and

combinations of the aforementioned non-hydrocarbon, oxygen-containing linking groups with an alkylene group or a fluorinated alkylene group.

The alkylene group or fluorinated alkylene group for Q³ is preferably a linear or branched group. Further, the alkylene group or fluorinated alkylene group preferably has 1 to 12 carbon atoms, more preferably 1 to 5 carbon atoms, still more preferably 1 to 4 carbon atoms, and most preferably 1 to 3 carbon atoms.

Specific examples of alkylene groups for Q³ include a methylene group [—CH₂—]; alkylmethylene groups such as —CH(CH₃)—, —CH(CH₂CH₃)—, —C(CH₃)₂—, —C(CH₃)(CH₂CH₃)—, —CH(CH₂CH₂CH₃)—, —C(CH₃)(CH₂CH₂CH₃)—, and —C(CH₂CH₃)₂—; an ethylene group [—CH₂CH₂—]; alkylethylene groups, such as —CH(CH₃)CH₂—, —CH(CH₃)CH(CH₃)—, —C(CH₃)₂CH₂—, —CH(CH₂CH₃)CH₂—, and —CH(CH₂CH₃)CH₂—; a trimethylene group (an n-propylene group) [—CH₂CH₂CH₂—]; alkyltrimethylene groups, such as —CH(CH₃)CH₂CH₂—, and —CH₂CH(CH₃)CH₂—; a tetramethylene group [—CH₂CH₂CH₂CH₂—]; alkyltetramethylene groups, such as —CH(CH₃)CH₂CH₂CH₂—, and —CH₂CH(CH₃)CH₂CH₂—; and a pentamethylene group [—CH₂CH₂CH₂CH₂CH₂—].

As the fluorinated alkylene group for Q³, groups in which part or all of the hydrogen atoms of the aforementioned alkylene groups for Q³ have been substituted with a fluorine atom can be mentioned, and specific examples include —CF₂—, —CF₂CF₂—, —CF₂CF₂CF₂—, —CF(CF₃)CF₂—, —CF(CF₂CF₃)—, —C(CF₃)₂—, —CF₂CF₂CF₂CF₂—, —CF(CF₃)CF₂CF₂—, —CF₂CF(CF₃)CF₂—, —CF(CF₃)CF(CF₃)—, —C(CF₃)₂CF₂—, —CF(CF₂CF₃)CF₂—, —CF(CF₂CF₂CF₃)—, —C(CF₃)(CF₂CF₃)—; —CHF—, —CH₂CF₂—, —CH₂CH₂CF₂—, —CH₂CF₂CF₂—, —CH(CF₃)CH₂—, —CH(CF₂CF₃)—, —C(CH₃)(CF₃)—, —CH₂CH₂CH₂CF₂—, —CH₂CH₂CF₂CF₂—, —CH(CF₃)CH₂CH₂—, —CH₂CH(CF₃)CH₂—, —CH(CF₃)CH(CF₃)—, and —C(CF₃)₂CH₂—.

Examples of combinations of the aforementioned non-hydrocarbon, oxygen-containing linking groups with an alkylene group or a fluorinated alkylene group include —R⁹¹—O—, —C(═O)—O—R⁹²—, —C(═O)—O—R⁹³—O—, and —R⁹⁴—C(═O)—O—R⁹⁵—O—. In the formulas, each of R⁹¹ to R⁹⁵ independently represents an alkylene group or a fluorinated alkylene group, and specific examples thereof include the same alkylene groups or fluorinated alkylene groups as those described above for Q³.

When Y¹⁰ (described later) in general formula (3) represents —SO₂—, it is particularly desirable that a carbon atom of Q³ bonded to the sulfur atom within Y¹⁰ be fluorinated. In such a case, an acid having a strong acid strength is generated from the component (B1) upon exposure. As a result, a resist pattern with an excellent shape can be formed, and various lithography properties such as EL margin and the like can be improved.

In general formula (3), when Y¹⁰ (described later) represents —SO₂—, the acid strength of the acid generated upon exposure can be controlled by adjusting the number of fluorine atoms within Q³. When the carbon atom is not fluorinated, although the acid strength becomes weak, improvement in roughness and the like can be expected.

The alkylene group or the fluorinated alkylene group for Q³ may have a substituent. The alkylene group or fluorinated alkylene group “has a substituent” means that part or all of the hydrogen atoms or fluorine atoms in the alkylene group or fluorinated alkylene group has been substituted with groups other than hydrogen atoms and fluorine atoms.

Examples of substituents for the alkylene group or fluorinated alkylene group include an alkoxy group of 1 to 4 carbon atoms, and a hydroxyl group.

As Q³, a single bond, an alkylene group, a fluorinated alkylene group or a divalent linking group containing an ether bond is preferable, and a single bond, an alkylene group or —R⁹¹—O— is particularly desirable.

In general formula (3), Y¹⁰ represents —C(═O)— or —SO₂—.

In general formula (3), Y¹¹ represents an alkyl group of 1 to 10 carbon atoms which may have a substituent or a fluorinated alkyl group of 1 to 10 carbon atoms which may have a substituent.

Y¹¹ has 1 to 10 carbon atoms, preferably 1 to 8 carbon atoms, and more preferably 1 to 4 carbon atoms.

Y¹¹ is preferably a fluorinated alkyl group which may have a substituent because the acid strength of the generated acid becomes stronger. The fluorination ratio (percentage (%) of the number of fluorine atoms, base on the total number of fluorine atoms and hydrogen atoms) is preferably 50 to 100%, more preferably 80 to 100%, and still more preferably 85 to 100%.

Further, when Y¹¹ is a fluorinated alkyl group, the skeleton “Y¹¹—SO₂—” exhibits excellent decomposability as compared to a perfluoroalkyl chain of 6 to 10 carbon atoms which is hardly decomposable, and bioaccumulation can be minimized to improve ease in handling. Furthermore, the fluorinated alkyl group is preferable in that the acid-generator component (B) can be uniformly distributed within a resist film.

The alkyl group or fluorinated alkyl group for Y¹¹ may have a substituent. Examples of substituents include an alkoxy group, a halogen atom other than fluorine, a halogenated alkyl group, a hydroxyl group and an oxygen atom (═O).

The alkoxy group as the substituent is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the halogen atom (other than fluorine) as the substituent include a chlorine atom, a bromine atom and an iodine atom.

Examples of the halogenated alkyl group as the substituent include groups in which part or all of the hydrogen atoms within the aforementioned alkyl groups has been substituted with the aforementioned halogen atoms.

Specific examples of anion moieties preferable as the anion moiety (3) are shown below.

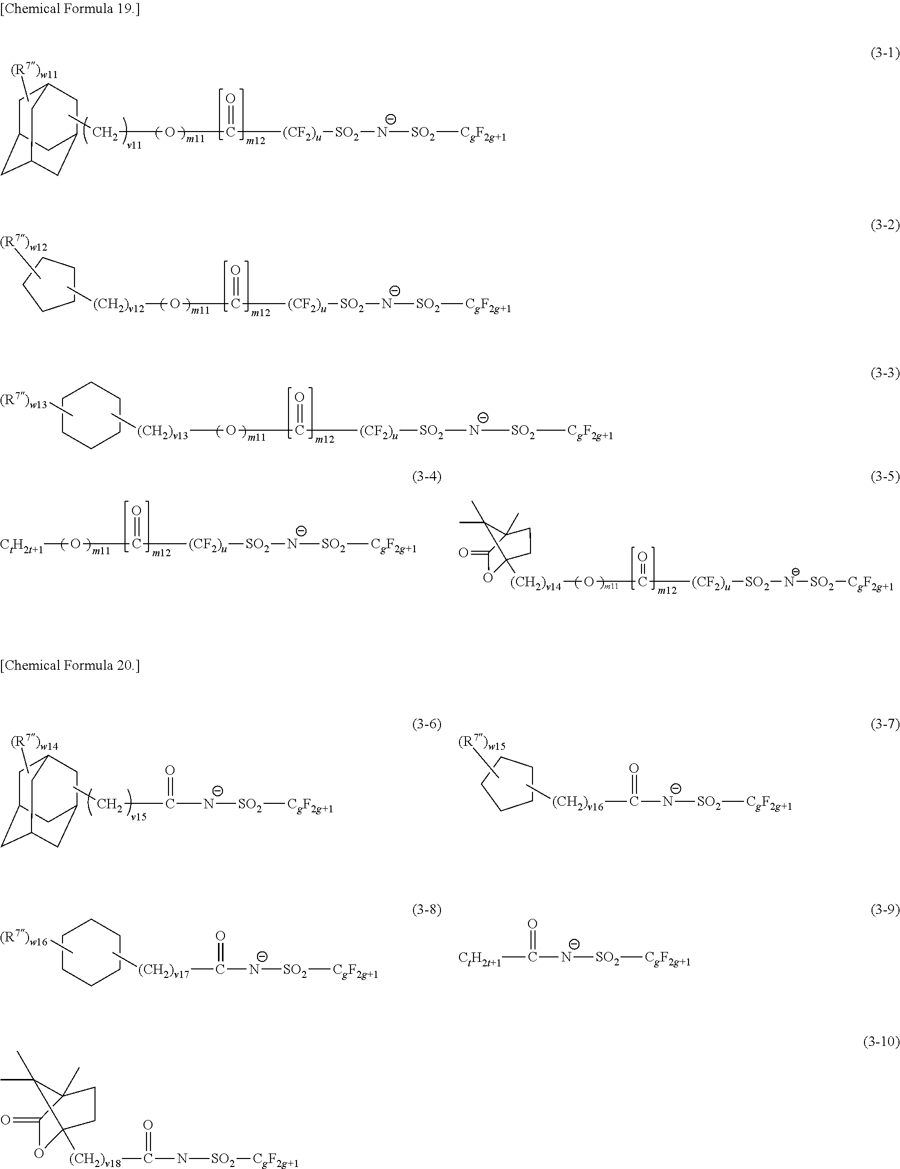

In the formulas, R⁷″ represents a substituent; each of w11 to w16 independently represents an integer of 0 to 3; each of v11 to v18 independently represents an integer of 0 to 3; u represents an integer of 0 to 4; m11 to m12 represents 0 or 1; g represents an integer of 1 to 4; and t represents an integer of 3 to 20.

In the formulas above, the substituent for R⁷″ is the same as defined above.

If there are two or more of the R⁷″ group, as indicated by the values w11 to w16, then the two or more of the R⁷″ groups may be the same or different from each other.

Each of w11 to w16 independently represents an integer of 0 to 3, preferably 0 or 1, and most preferably 0.

Each of v11 to v18 independently represents an integer of 0 to 3, and more preferably 0 or 1.

Each u independently represents an integer of 0 to 4, and preferably 0 to 2.

Each g independently represents an integer of 1 to 4, preferably 1 or 2, and most preferably 1.

t represents an integer of 3 to 20, preferably 3 to 15, and more preferably 3 to 12.

{Anion Moiety (4)}

In general formula (4), Y¹² represents a cyclic alkyl group of 4 to 12 carbon atoms which may have an oxygen atom (═O) as a substituent.

The expression “may have an oxygen atom (═O) as a substituent” means that two hydrogen atoms bonded to a carbon atom constituting the cyclic alkyl group of 4 to 20 carbon atoms may be substituted with an oxygen atom (═O).

The cyclic alkyl group represented by Y¹² is not particularly limited as long as it has 4 to 20 carbon atoms, and may be either polycyclic or monocyclic. Examples thereof include a group in which one hydrogen atom has been removed from a monocycloalkane or a polycycloalkane such as a bicycloalkane, tricycloalkane or tetracycloalkane. As the monocyclic group, a group in which one hydrogen atom has been removed from a monocycloalkane of 3 to 8 carbon atoms is preferable, and specific examples thereof include a cyclopentyl group, a cyclohexyl group, a cycloheptyl group and a cyclooctyl group. The polycyclic group preferably has 7 to 12 carbon atoms, and specific examples thereof include an adamantyl group, a norbornyl group, an isobornyl group, a tricyclodecyl group and a tetracyclododecyl group. From an industrial viewpoint, an adamantyl group, a norbornyl group or a tetracyclododecyl group is particularly desirable.

When the Y¹² group has an oxygen atom (═O) as a substituent, it preferably has 1 or 2 substituents, and most preferably 1 substituent.

When Y¹² has an oxygen atom (═O) as a substituent, the substituent is preferably bonded to a carbon atom adjacent to the carbon atom on the terminal of the group “—(CH₂)_{q}—SO₃”.

Y¹² may have a substituent other than oxygen. As an example of such a substituent, an alkyl group of 1 to 5 carbon atoms can be given. The alkyl group of 1 to 5 carbon atoms is preferably linear or branched, and preferably has 1 to 3 carbon atoms. As the lower alkyl group, a methyl group is particularly desirable.

When Y¹² has a substituent other than an oxygen atom (═O), it preferably has 1 to 3 substituents, and more preferably 1 or 2 substituents.

When Y¹² has a substituent other than an oxygen atom (═O), the bonding position of the substituent is the same as in the case of the oxygen atom (═O).

As Y¹², a cyclic group of 4 to 12 carbon atoms which has an oxygen atom (═O) as a substituent is preferable, a polycyclic alkyl group of 4 to 20 carbon atoms which has an oxygen atom (═O) as a substituent is more preferable, an adamantyl group, a norbornyl group or a tetracyclododecyl group which has an oxygen atom (═O) as a substituent is still more preferable, and a norbornyl group which has an oxygen atom (═O) as a substituent is particularly desirable.

In general formula (4), q represents 0 or 1, and preferably 1.

In terms of the effects of the present invention, the anion moiety (4) is preferably a camphorsulfonate ion, and it is particularly desirable that the camphorsulfonate ion is represented by the formula (4-1) shown below (a group in which the sulfonate ion (—SO₃⁻) is bonded to the carbon atom of the methyl group bonded to the first position of the norbornane ring).

Further, an ion represented by the formula (4-2) shown below can also be given as a preferable example.

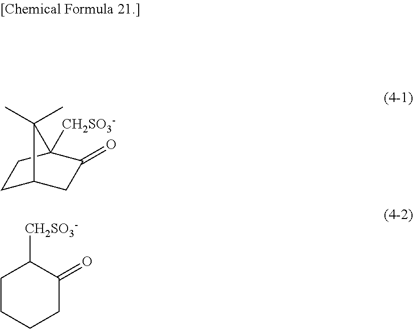

<<Acid Generator>>

The acid generator according to a fourth aspect of the present invention (hereafter, frequently referred to as “acid generator (B1)”) is an acid generator including the compound (b1). In the formula, R¹, R², x, n, and X⁻ are the same as defined for the compound according to the third aspect of the present invention.

<<Resist Composition>>

Next, the resist composition according to the first aspect of the present invention will be described.

The resist composition according to the first aspect of the present invention includes a base component (A) which exhibits changed solubility in an alkali developing solution under action of acid (hereafter, referred to as “component (A)”) and an acid-generator component (B) which generates acid upon exposure (hereafter, referred to as “component (B)”), and the component (B) contains an acid generator (B1) including the compound represented by general formula (b1).

In the resist composition of the present invention, as the component (A), a polymeric material which exhibits changed solubility in an alkali developing solution under action of acid may be used. Alternatively, as the component (A), a low molecular weight material which exhibits changed solubility in an alkali developing solution under action of acid may be used.

Further, the resist composition of the present invention may be either a negative resist composition or a positive resist composition.

When the resist composition of the present invention is a negative resist composition, for example, the component (A) is an alkali-soluble resin, and a cross-linking agent (C) is blended in the negative resist composition.

In the negative resist composition, when acid is generated from the component (B) upon exposure in the formation of a resist pattern, the action of the generated acid causes cross-linking between the alkali-soluble resin and the cross-linking agent at exposed portions, and the cross-linked portion becomes alkali-insoluble.

As the alkali-soluble resin, it is preferable to use a resin having structural units derived from at least one of an α-(hydroxyalkyl)acrylic acid and an alkyl ester of an α-(hydroxyalkyl)acrylic acid having 1 to 5 carbon atoms, as such resins enable the formation of a satisfactory resist pattern with minimal swelling. Here, the term “α-(hydroxyalkyl)acrylic acid” refers to one or both of acrylic acid in which a hydrogen atom is bonded to the carbon atom on the α-position having the carboxyl group bonded thereto, and α-hydroxyalkylacrylic acid in which a hydroxyalkyl group (preferably a hydroxyalkyl group of 1 to 5 carbon atoms) is bonded to the carbon atom on the α-position.

As the cross-linking agent (C), typically, an amino-based cross-linking agent such as a glycoluril having a methylol group or alkoxymethyl group is preferable, as it enables formation of a resist pattern with minimal swelling. The amount of the cross-linking agent (C) added is preferably within a range from 1 to 50 parts by weight, relative to 100 parts by weight of the alkali-soluble resin.

When the resist composition of the present invention is a positive resist composition, the component (A) is insoluble in an alkali developing solution prior to exposure, and when acid is generated from the component (B) upon exposure in the formation of a resist pattern, acid dissociable, dissolution inhibiting groups are dissociated, and the solubility of the component (A) in an alkali developing solution increases. As a result, the positive resist composition changes from an alkali-insoluble state to an alkali-soluble state. Therefore, in the formation of a resist pattern, by conducting selective exposure of a resist film formed by applying the positive resist composition to a substrate, the exposed portions changes from an alkali-insoluble state to an alkali-soluble state, whereas the unexposed portions remain alkali-insoluble, and hence, a resist pattern can be formed by alkali developing.

In the resist composition of the present invention, the component (A) is preferably a base component that exhibits increased solubility in an alkali developing solution under the action of acid. That is, the resist composition of the present invention is preferably a positive resist composition. The component (A) contains a resin component (A1) (hereafter, referred to as “component (A1)”) which exhibits increased alkali solubility by the action of acid.

<Component (A1)>

In the positive resist composition, it is preferable that the component (A1) include a structural unit (a1) derived from an acrylate ester containing an acid dissociable, dissolution inhibiting group.

It is preferable that the component (A1) further include a structural unit (a2) derived from an acrylate ester containing a lactone-containing cyclic group.

It is preferable that the component (A1) further include a structural unit (a3) derived from an acrylate ester containing a polar group-containing aliphatic hydrocarbon group.

In the present descriptions and claims, the term “structural unit derived from an acrylate ester” refers to a structural unit which is formed by the cleavage of the ethylenic double bond of an acrylate ester.

The term “acrylate ester” is a generic term that includes acrylate esters having a hydrogen atom bonded to the carbon atom on the α-position, and acrylate esters having a substituent (an atom other than a hydrogen atom or a group) bonded to the carbon atom on the α-position.

Examples of the substituent include an alkyl group of 1 to 5 carbon atoms and a halogenated alkyl group of 1 to 5 carbon atoms. With respect to the “structural unit derived from an acrylate ester”, the “α-position (the carbon atom on the α-position)” refers to the carbon atom having the carbonyl group bonded thereto, unless specified otherwise.

With respect to the acrylate ester, specific examples of the alkyl group of 1 to 5 carbon atoms for the substituent at the α-position include linear or branched alkyl groups of 1 to 5 carbon atoms such as a methyl group, an ethyl group, a propyl group, an isopropyl group, an n-butyl group, an isobutyl group, a tert-butyl group, a pentyl group, an isopentyl group, and a neopentyl group.

Specific examples of the halogenated alkyl group of 1 to 5 carbon atoms include groups in which part or all of the hydrogen atoms of the aforementioned “alkyl group of 1 to 5 carbon atoms for the substituent at the α-position” are substituted with halogen atoms. Examples of halogen atoms include fluorine atoms, chlorine atoms, bromine atoms and iodine atoms, and fluorine atoms are particularly desirable.

In the present invention, it is preferable that a hydrogen atom, an alkyl group of 1 to 5 carbon atoms or a halogenated alkyl group of 1 to 5 carbon atoms is bonded to the α-position of the acrylate ester, a hydrogen atom, an alkyl group of 1 to 5 carbon atoms or a fluorinated alkyl group of 1 to 5 carbon atoms is more preferable, and in terms of industrial availability, a hydrogen atom or a methyl group is the most desirable.

Structural Unit (a1)

The structural unit (a1) is a structural unit derived from an acrylate ester containing an acid dissociable, dissolution inhibiting group.

As the acid dissociable, dissolution inhibiting group in the structural unit (a1), any of the groups that have been proposed as acid dissociable, dissolution inhibiting groups for the base resins of chemically amplified resists can be used, provided the group has an alkali dissolution-inhibiting effect that renders the entire component (A1) insoluble in an alkali developing solution prior to dissociation, and then following dissociation by action of acid, increases the solubility of the entire component (A1) in the alkali developing solution.

Generally, groups that form either a cyclic or chain-like tertiary alkyl ester with the carboxyl group of the (meth)acrylic acid, and acetal-type acid dissociable, dissolution inhibiting groups such as alkoxyalkyl groups are widely known. Here, the term “(meth)acrylate ester” is a generic term that includes either or both of the acrylate ester having a hydrogen atom bonded to the α-position and the methacrylate ester having a methyl group bonded to the α-position.

Here, a tertiary alkyl ester describes a structure in which an ester is formed by substituting the hydrogen atom of a carboxyl group with a chain-like or cyclic tertiary alkyl group, and a tertiary carbon atom within the chain-like or cyclic tertiary alkyl group is bonded to the oxygen atom at the terminal of the carbonyloxy group (—C(O)—O—). In this tertiary alkyl ester, the action of acid causes cleavage of the bond between the oxygen atom and the tertiary carbon atom.

The chain-like or cyclic alkyl group may have a substituent.

Hereafter, for the sake of simplicity, groups that exhibit acid dissociability as a result of the formation of a tertiary alkyl ester with a carboxyl group are referred to as “tertiary alkyl ester-type acid dissociable, dissolution inhibiting groups”.

Examples of tertiary alkyl ester-type acid dissociable, dissolution inhibiting groups include aliphatic branched, acid dissociable, dissolution inhibiting groups and aliphatic cyclic group-containing acid dissociable, dissolution inhibiting groups.

In the present description and claims, the term “aliphatic” is a relative concept used in relation to the term “aromatic”, and defines a group or compound that has no aromaticity.

The term “aliphatic branched” refers to a branched structure having no aromaticity.

The “aliphatic branched, acid dissociable, dissolution inhibiting group” is not limited to be constituted of only carbon atoms and hydrogen atoms (not limited to hydrocarbon groups), but is preferably a hydrocarbon group.

Further, the “hydrocarbon group” may be either saturated or unsaturated, but is preferably saturated.

Examples of aliphatic branched, acid dissociable, dissolution inhibiting groups include tertiary alkyl groups of 4 to 8 carbon atoms, and specific examples include a tert-butyl group, tert-pentyl group and tert-heptyl group.

The term “aliphatic cyclic group” refers to a monocyclic group or polycyclic group that has no aromaticity.

The “aliphatic cyclic group” within the structural unit (a1) may or may not have a substituent. Examples of the substituent include an alkyl group of 1 to 5 carbon atoms, a fluorine atom, a fluorinated alkyl group of 1 to 5 carbon atoms, and an oxygen atom (═O).

The basic ring of the “aliphatic cyclic group” exclusive of substituents is not limited to be constituted from only carbon and hydrogen (not limited to hydrocarbon groups), but is preferably a hydrocarbon group. Further, the “hydrocarbon group” may be either saturated or unsaturated, but is preferably saturated. Furthermore, the “aliphatic cyclic group” is preferably a polycyclic group.

As such aliphatic cyclic groups, groups in which one or more hydrogen atoms have been removed from a monocycloalkane or a polycycloalkane such as a bicycloalkane, tricycloalkane or tetracycloalkane which may or may not be substituted with an alkyl group of 1 to 5 carbon atoms, a fluorine atom or a fluorinated alkyl group, may be used. Examples of such groups include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane.

As the aliphatic cyclic group-containing acid dissociable, dissolution inhibiting group, for example, a group which has a tertiary carbon atom on the ring structure of the cycloalkyl group can be used. Specific examples include 2-methyl-2-adamantyl group and a 2-ethyl-2-adamantyl group. Further, groups having an aliphatic cyclic group such as an adamantyl group, cyclohexyl group, cyclopentyl group, norbornyl group, tricyclodecyl group or tetracyclododecyl group, and a branched alkylene group having a tertiary carbon atom bonded thereto, as the groups bonded to the oxygen atom of the carbonyl group (—C(O)—O—) within the structural units represented by general formulas (a1″-1) to (a1″-6) shown below, can be used.

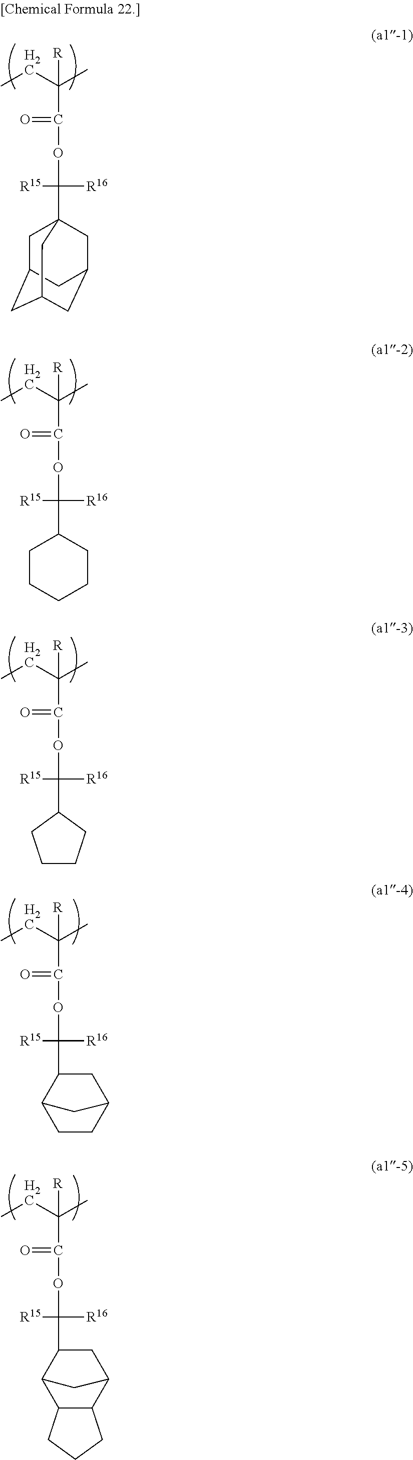

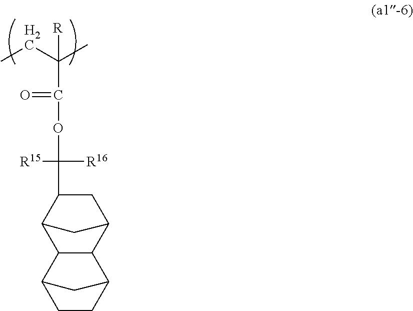

In the formulas, R represents a hydrogen atom, an alkyl group of 1 to 5 carbon atoms or a halogenated alkyl group of 1 to 5 carbon atoms; and R¹⁵ and R¹⁶ each independently represent an alkyl group (which may be linear or branched, and preferably has 1 to 5 carbon atoms).

In general formulas (a1″-1) to (a1″-6) above, the alkyl group of 1 to 5 carbon atoms or halogenated alkyl group of 1 to 5 carbon atoms for R are the same as the alkyl group of 1 to 5 carbon atoms or halogenated alkyl group of 1 to 5 carbon atoms which can be bonded to the α-position of the aforementioned acrylate ester.

An “acetal-type acid dissociable, dissolution inhibiting group” generally substitutes a hydrogen atom at the terminal of an alkali-soluble group such as a carboxy group or hydroxyl group, so as to be bonded with an oxygen atom. When acid is generated upon exposure, the generated acid acts to break the bond between the acetal-type acid dissociable, dissolution inhibiting group and the oxygen atom to which the acetal-type, acid dissociable, dissolution inhibiting group is bonded.

Examples of acetal-type acid dissociable, dissolution inhibiting groups include groups represented by general formula (p1) shown below.

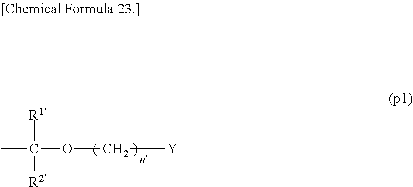

In the formula, R¹′ and R²′ each independently represent a hydrogen atom or an alkyl group of 1 to 5 carbon atoms; n′ represents an integer of 0 to 3; and Y represents an alkyl group of 1 to 5 carbon atoms or an aliphatic cyclic group.

In general formula (p1) above, n′ is preferably an integer of 0 to 2, more preferably 0 or 1, and most preferably 0.

As the alkyl group of 1 to 5 carbon atoms for R¹′ and R²′, the same alkyl groups of 1 to 5 carbon atoms as those described above for R can be used, although a methyl group or ethyl group is preferable, and a methyl group is particularly desirable.

In the present invention, it is preferable that at least one of R¹′ and R²′ be a hydrogen atom. That is, it is preferable that the acid dissociable, dissolution inhibiting group (p1) is a group represented by general formula (p1-1) shown below.

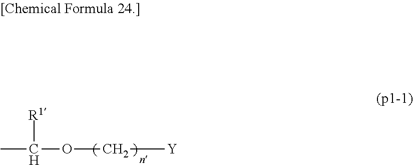

In the formula, R¹′, n′ and Y are the same as defined above.

As the alkyl group of 1 to 5 carbon atoms for Y, the same alkyl groups of 1 to 5 carbon atoms as those described above can be used.

As the aliphatic cyclic group for Y, any of the aliphatic monocyclic/polycyclic groups which have been proposed for conventional ArF resists and the like can be appropriately selected for use. For example, the same groups described above in connection with the “aliphatic cyclic group” can be used.

Further, as the acetal-type, acid dissociable, dissolution inhibiting group, groups represented by general formula (p2) shown below can also be used.

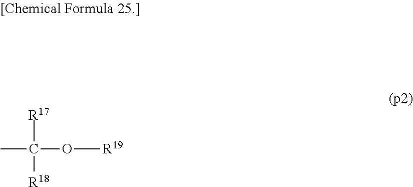

In the formula, R¹⁷ and R¹⁸ each independently represent a linear or branched alkyl group or a hydrogen atom; and R¹⁹ represents a linear, branched or cyclic alkyl group; or R¹⁷ and R¹⁹ each independently represents a linear or branched alkylene group, and the terminal of R¹⁷ is bonded to the terminal of R¹⁹ to form a ring.

The alkyl group for R¹⁷ and R¹⁸ preferably has 1 to 15 carbon atoms, and may be either linear or branched. As the alkyl group, an ethyl group or a methyl group is preferable, and a methyl group is most preferable.

It is particularly desirable that either one of R¹⁷ and R¹⁸ be a hydrogen atom, and the other be a methyl group.

R¹⁹ represents a linear, branched or cyclic alkyl group which preferably has 1 to 15 carbon atoms, and may be any of linear, branched or cyclic.

When R¹⁹ represents a linear or branched alkyl group, it is preferably an alkyl group of 1 to 5 carbon atoms, more preferably an ethyl group or methyl group, and most preferably an ethyl group.

When R¹⁹ represents a cycloalkyl group, it preferably has 4 to 15 carbon atoms, more preferably 4 to 12 carbon atoms, and most preferably 5 to 10 carbon atoms. As examples of the cycloalkyl group, groups in which one or more hydrogen atoms have been removed from a monocycloalkane or a polycycloalkane such as bicycloalkane, tricycloalkane or tetracycloalkane, which may or may not be substituted with a fluorine atom or a fluorinated alkyl group, may be used. Examples of such groups include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane. Among these, a group in which one or more hydrogen atoms have been removed from adamantane is preferable.

In general formula (p2) above, R¹⁷ and R¹⁹ may each independently represent a linear or branched alkylene group (preferably an alkylene group of 1 to 5 carbon atoms), and the terminal of R¹⁹ may be bonded to the terminal of R¹⁷.

In such a case, a cyclic group is formed by R¹⁷, R¹⁹, the oxygen atom having R¹⁹ bonded thereto, and the carbon atom having the oxygen atom and R¹⁷ bonded thereto. Such a cyclic group is preferably a 4- to 7-membered ring, and more preferably a 4- to 6-membered ring. Specific examples of the cyclic group include tetrahydropyranyl group and tetrahydrofuranyl group.

As the structural unit (a1), it is preferable to use at least one member selected from the group consisting of structural units represented by formula (a1-0-1) shown below and structural units represented by formula (a1-0-2) shown below.

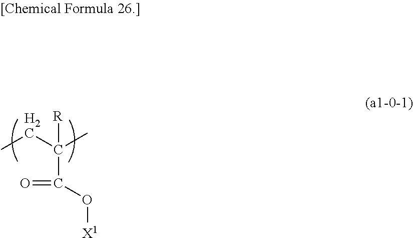

In the formula, R represents a hydrogen atom, an alkyl group of 1 to 5 carbon atoms or a halogenated alkyl group of 1 to 5 carbon atoms; and X¹ represents an acid dissociable, dissolution inhibiting group.

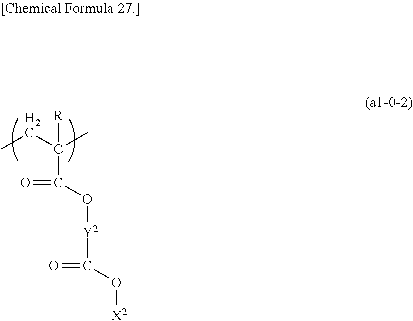

In the formula, R represents a hydrogen atom, an alkyl group of 1 to 5 carbon atoms or a halogenated alkyl group of 1 to 5 carbon atoms; X² represents an acid dissociable, dissolution inhibiting group; and Y² represents a divalent linking group.

In general formula (a1-0-1) above, the alkyl group of 1 to 5 carbon atoms or halogenated alkyl group of 1 to 5 carbon atoms for R are the same as the alkyl group of 1 to 5 carbon atoms or halogenated alkyl group of 1 to 5 carbon atoms which can be bonded to the α-position of the aforementioned acrylate ester.

X¹ is not particularly limited as long as it is an acid dissociable, dissolution inhibiting group. Examples thereof include the aforementioned tertiary alkyl ester-type acid dissociable, dissolution inhibiting groups and acetal-type acid dissociable, dissolution inhibiting groups, and tertiary alkyl ester-type acid dissociable, dissolution inhibiting groups are preferable.

In general formula (a1-0-2), R is the same as defined above.

X² is the same as defined for X¹ in general formula (a1-0-1).

As the divalent linking group for Y², an alkylene group, a divalent aliphatic cyclic group or a divalent linking group containing a hetero atom can be mentioned.

As the aliphatic cyclic group, the same as those used above in connection with the explanation of “aliphatic cyclic group” can be used, except that two hydrogen atoms have been removed therefrom.

When Y² represents an alkylene group, it preferably has 1 to 10 carbon atoms, more preferably 1 to 6, still more preferably 1 to 4, and most preferably 1 to 3.

When Y² represents a divalent aliphatic cyclic group, it is particularly desirable that the divalent aliphatic cyclic group be a group in which two or more hydrogen atoms have been removed from cyclopentane, cyclohexane, norbornane, isobornane, adamantane, tricyclodecane or tetracyclododecane.

When Y² represents a divalent linking group containing a hetero atom, examples thereof include —O—, —C(═O)—O—, —C(═O)—, —O—C(═O)—O—, —C(═O)—NH—, —NH— (H may be substituted with a substituent such as an alkyl group or an acyl group), —S—, —S(═O)₂—, —S(═O)₂—O—, and “-A-O—B—(wherein O is an oxygen atom, and each of A and B independently represents a divalent hydrocarbon group which may have a substituent)”.

When Y² represents a divalent linking group —NH— and the H in the formula is replaced with a substituent such as an alkyl group or an acyl group, the substituent preferably has 1 to 10 carbon atoms, more preferably 1 to 8 carbon atoms, and most preferably 1 to 5 carbon atoms.

When Y² is “A-O—B”, each of A and B independently represents a divalent hydrocarbon group which may have a substituent.

A hydrocarbon “has a substituent” means that part or all of the hydrogen atoms within the hydrocarbon group is substituted with groups or atoms other than hydrogen atom.

The hydrocarbon group for A may be either an aliphatic hydrocarbon group, or an aromatic hydrocarbon group. An “aliphatic hydrocarbon group” refers to a hydrocarbon group that has no aromaticity.

The aliphatic hydrocarbon group for A may be either saturated or unsaturated. In general, the aliphatic hydrocarbon group is preferably saturated.

As specific examples of the aliphatic hydrocarbon group for A, a linear or branched aliphatic hydrocarbon group, and an aliphatic hydrocarbon group having a ring in the structure thereof can be given.

The linear or branched aliphatic hydrocarbon group preferably has 1 to 10 carbon atoms, more preferably 1 to 8, still more preferably 2 to 5, and most preferably 2.

As a linear aliphatic hydrocarbon group, a linear alkylene group is preferable, and specific examples include a methylene group, an ethylene group [—(CH₂)₂—], a trimethylene group [—(CH₂)₃—], a tetramethylene group [—(CH₂)₄—] and a pentamethylene group [—(CH₂)₅—].

As the branched aliphatic hydrocarbon group, a branched alkylene group is preferable, and specific examples include alkylalkylene groups, e.g., alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; alkylethylene groups such as —CH(CH₃)CH₂—, —CH(CH₃)CH(CH₃)—, —C(CH₃)₂CH₂— and —CH(CH₂CH₃)CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group within the alkylalkylene group, a linear alkyl group of 1 to 5 carbon atoms is preferable.

The linear or branched aliphatic hydrocarbon group (chain-like aliphatic hydrocarbon group) may or may not have a substituent. Examples of the substituent include a fluorine atom, a fluorinated alkyl group of 1 to 5 carbon atoms, and an oxygen atom (═O).

As examples of the hydrocarbon group containing a ring, a cyclic aliphatic hydrocarbon group (a group in which two hydrogen atoms have been removed from an aliphatic hydrocarbon ring), and a group in which the cyclic aliphatic hydrocarbon group is bonded to the terminal of the aforementioned chain-like aliphatic hydrocarbon group or interposed within the aforementioned chain-like aliphatic hydrocarbon group, can be given.

The cyclic aliphatic hydrocarbon group preferably has 3 to 20 carbon atoms, and more preferably 3 to 12 carbon atoms.

The cyclic aliphatic hydrocarbon group may be either a polycyclic group or a monocyclic group. As the monocyclic group, a group in which two hydrogen atoms have been removed from a monocycloalkane of 3 to 6 carbon atoms is preferable. Examples of the monocycloalkane include cyclopentane and cyclohexane.

As the polycyclic group, a group in which two hydrogen atoms have been removed from a polycycloalkane of 7 to 12 carbon atoms is preferable. Examples of the polycycloalkane include adamantane, norbornane, isobornane, tricyclodecane and tetracyclododecane.

The cyclic aliphatic hydrocarbon group may or may not have a substituent. Examples of the substituent include an alkyl group of 1 to 5 carbon atoms, a fluorine atom, a fluorinated alkyl group of 1 to 5 carbon atoms, and an oxygen atom (═O).

As A, a linear aliphatic hydrocarbon group is preferable, more preferably a linear alkylene group, still more preferably a linear alkylene group of 2 to 5 carbon atoms, and most preferably an ethylene group.

As the hydrocarbon group for B, the same divalent hydrocarbon groups as those described above for A can be used.

As B, a linear or branched aliphatic hydrocarbon group is preferable, and a methylene group or an alkylmethylene group is particularly desirable.

The alkyl group within the alkyl methylene group is preferably a linear alkyl group of 1 to 5 carbon atoms, more preferably a linear alkyl group of 1 to 3 carbon atoms, and most preferably a methyl group.

Specific examples of the structural unit (a1) include structural units represented by general formulas (a1-1) to (a1-4) shown below.

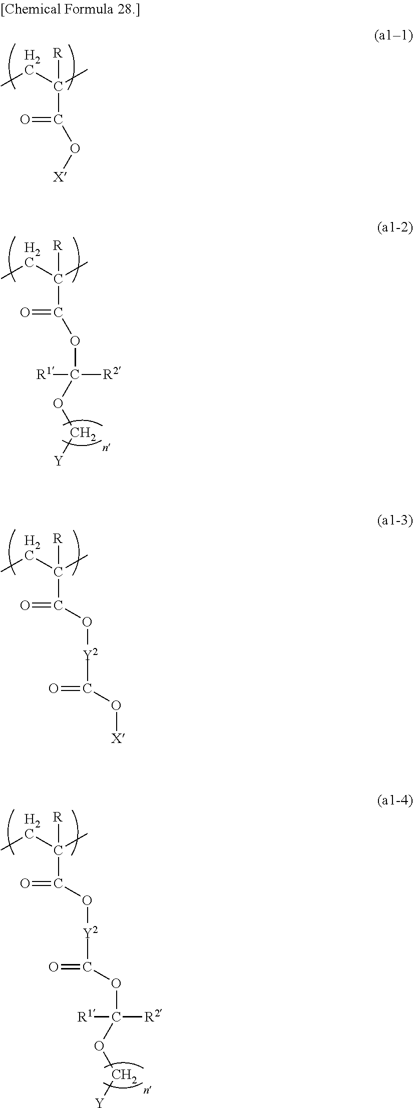

In the formulas, X′ represents a tertiary alkyl ester-type acid dissociable, dissolution inhibiting group; Y represents an alkyl group of 1 to 5 carbon atoms or an aliphatic cyclic group; n′ represents an integer of 0 to 3; Y² represents a divalent linking group; R is the same as defined above; and each of R¹′ and R²′ independently represents a hydrogen atom or an alkyl group of 1 to 5 carbon atoms.

Examples of the tertiary alkyl ester-type acid dissociable, dissolution inhibiting group for X′ include the same tertiary alkyl ester-type acid dissociable, dissolution inhibiting groups as those described above for X′.

As R¹′, R²′, n′ and Y are respectively the same as defined for R¹′, R²′, n′ and Y in general formula (p1) described above in connection with the “acetal-type acid dissociable, dissolution inhibiting group”.

As examples of Y², the same groups as those described above for Y² in general formula (a1-0-2) can be given.

Specific examples of structural units represented by general formula (a1-1) to (a1-4) are shown below.

In the formulas shown below, R^{α} represents a hydrogen atom, a methyl group or a trifluoromethyl group.

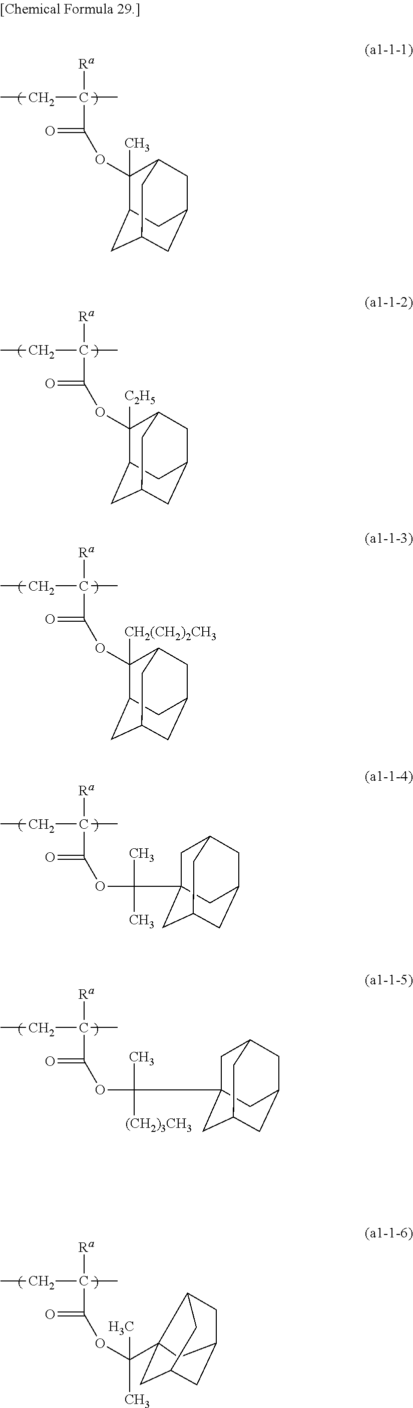

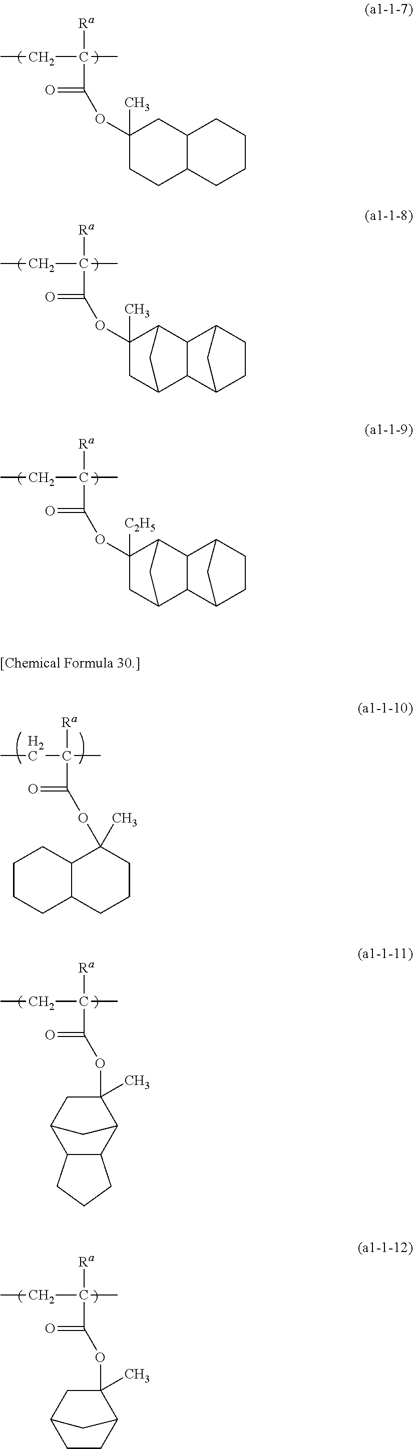

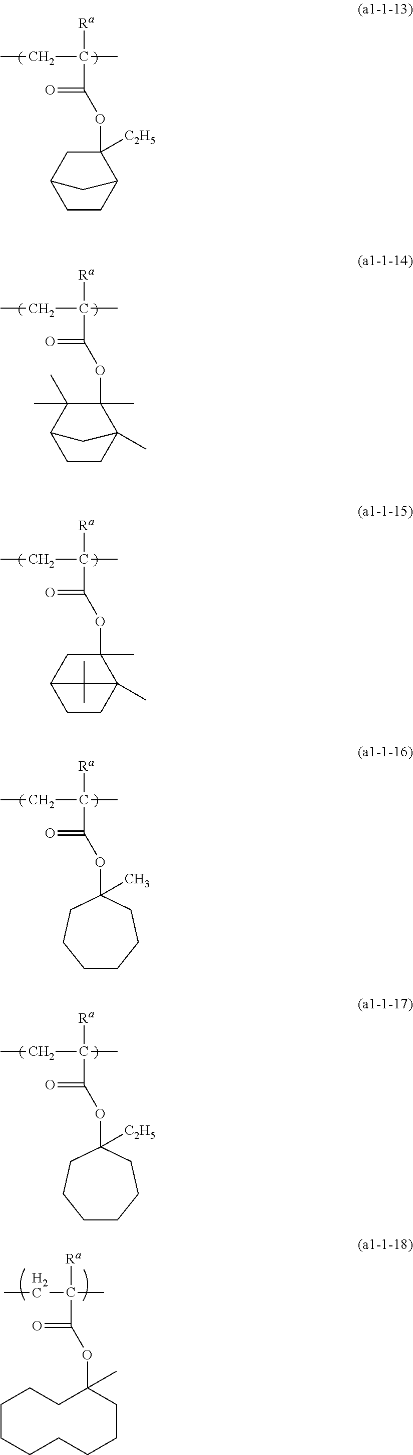

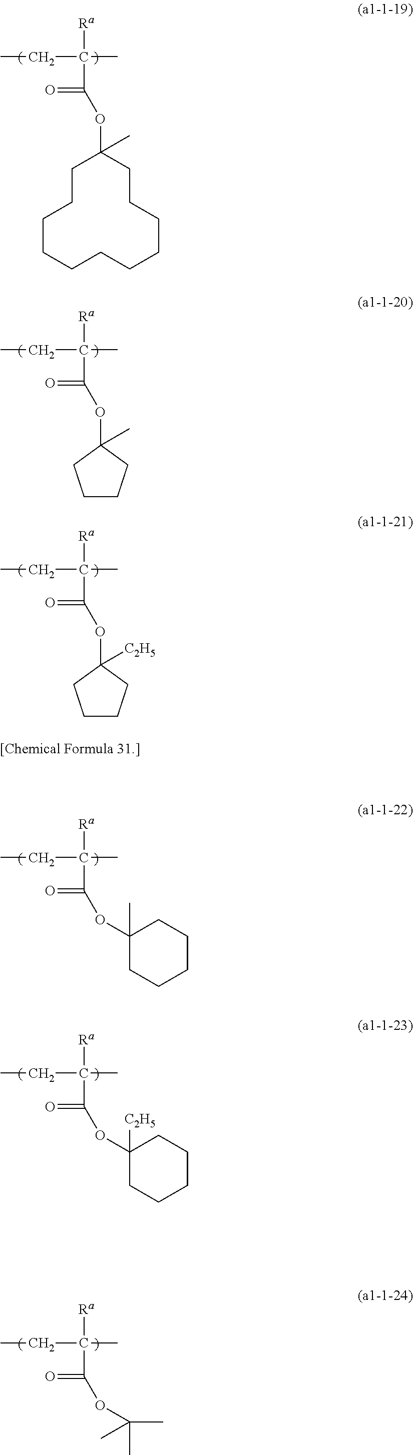

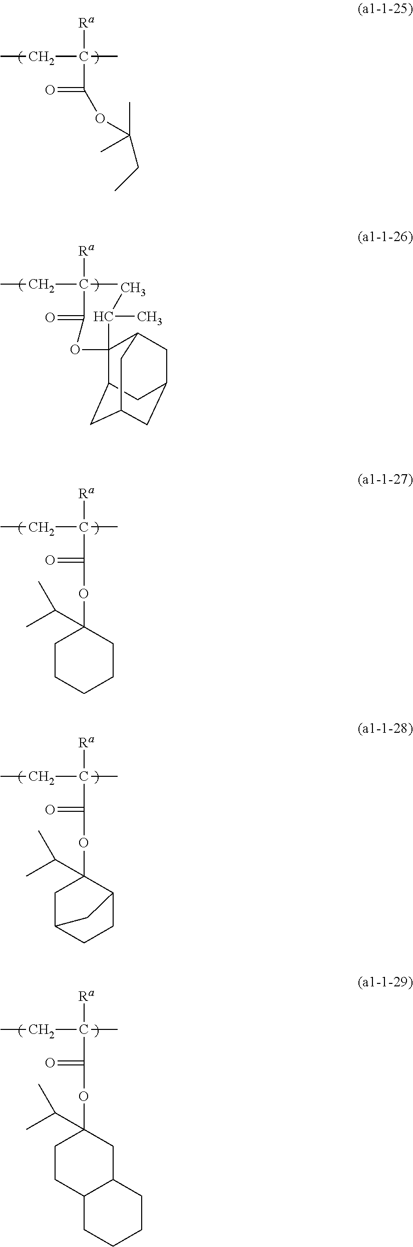

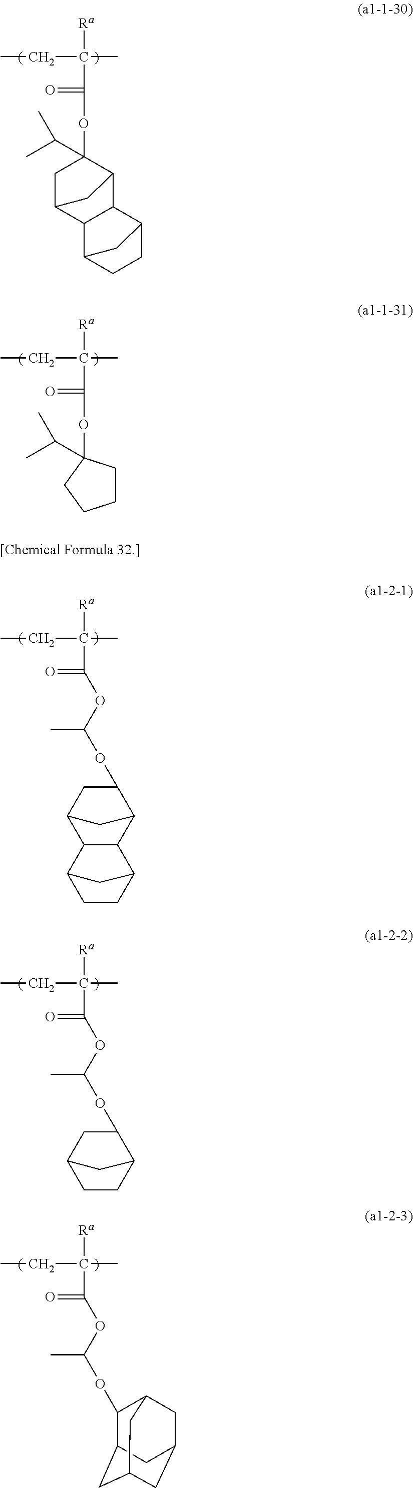

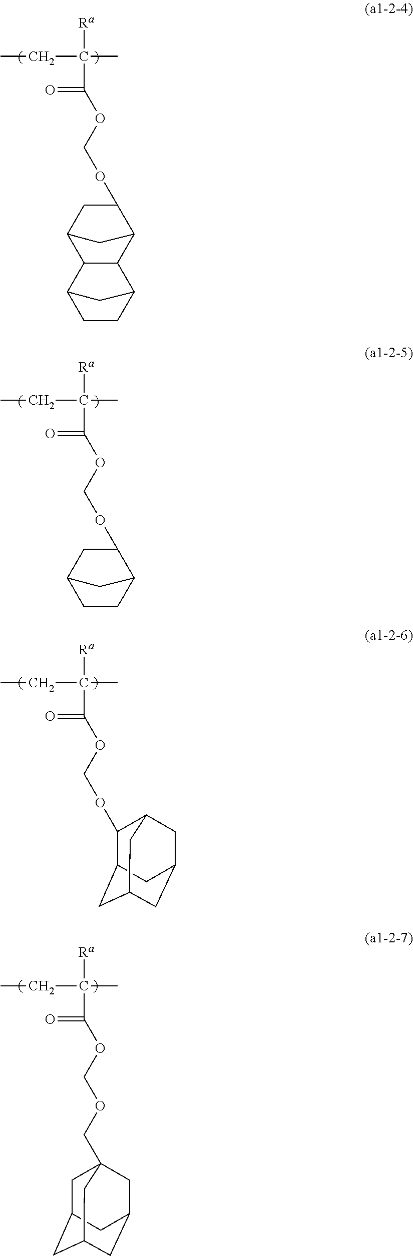

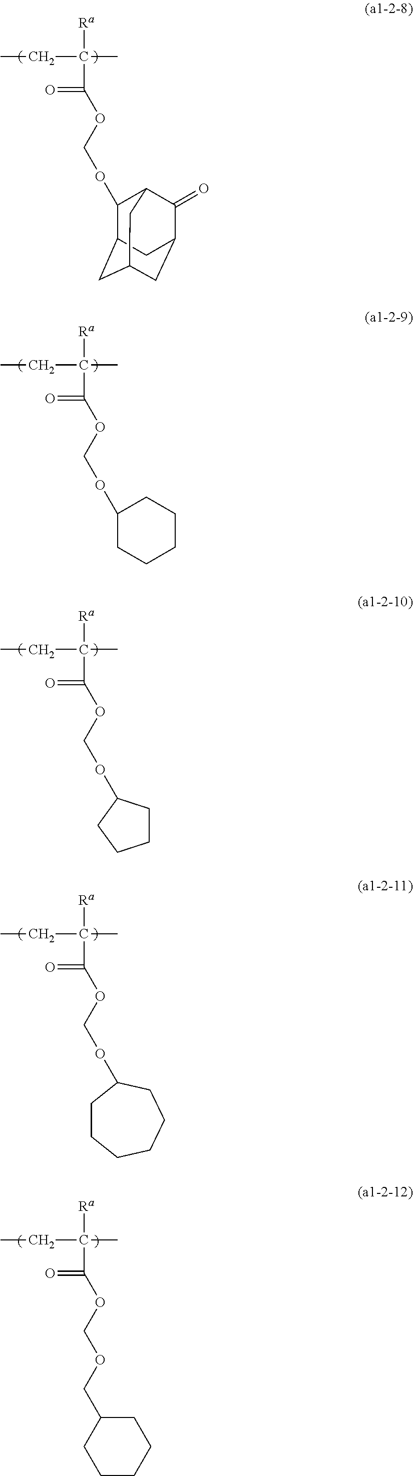

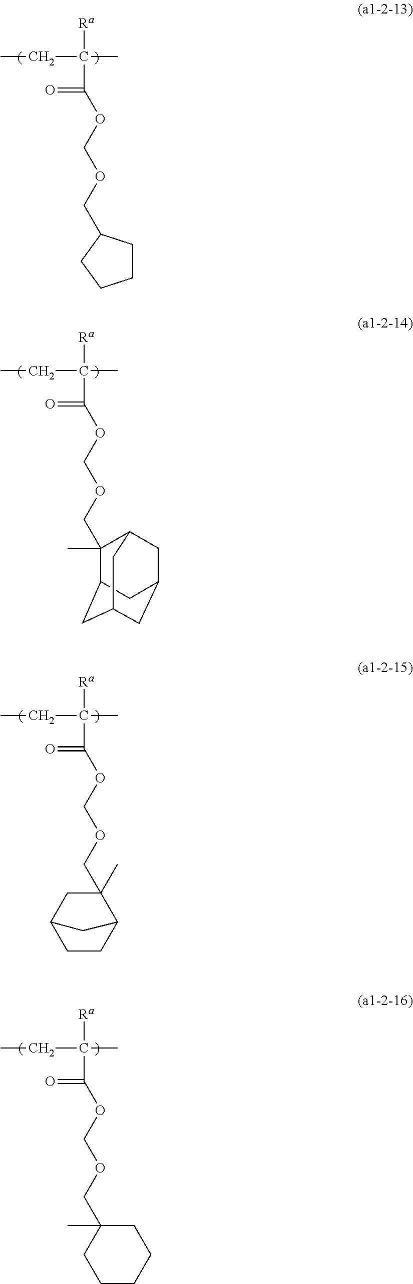

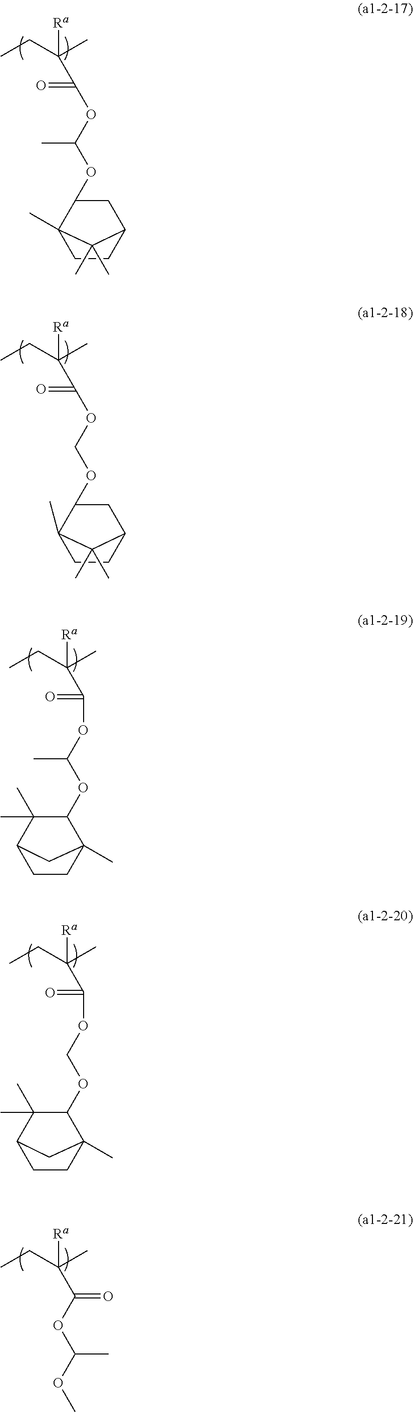

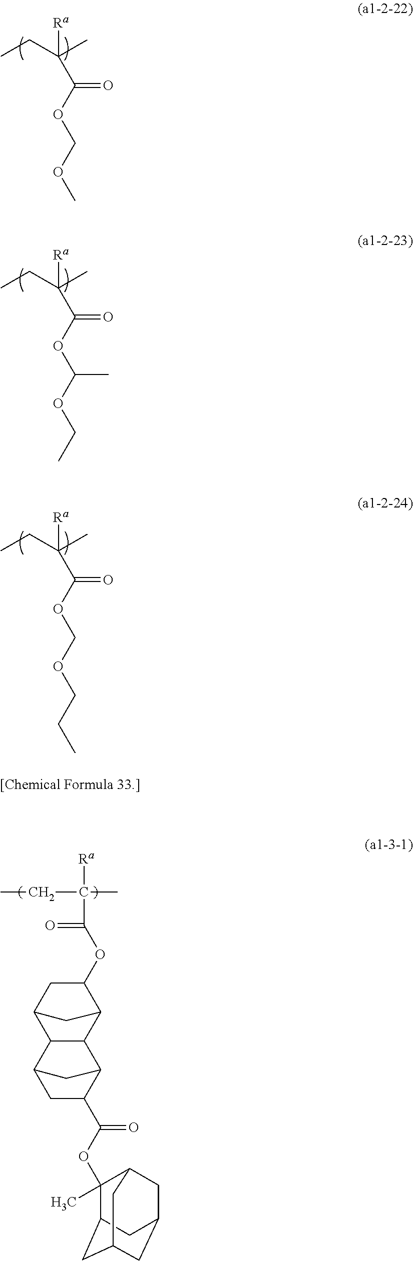

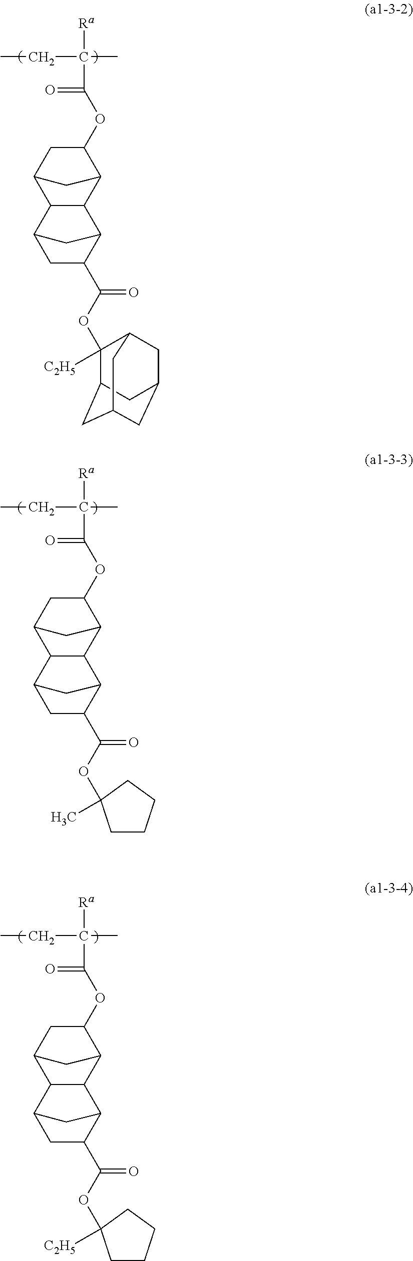

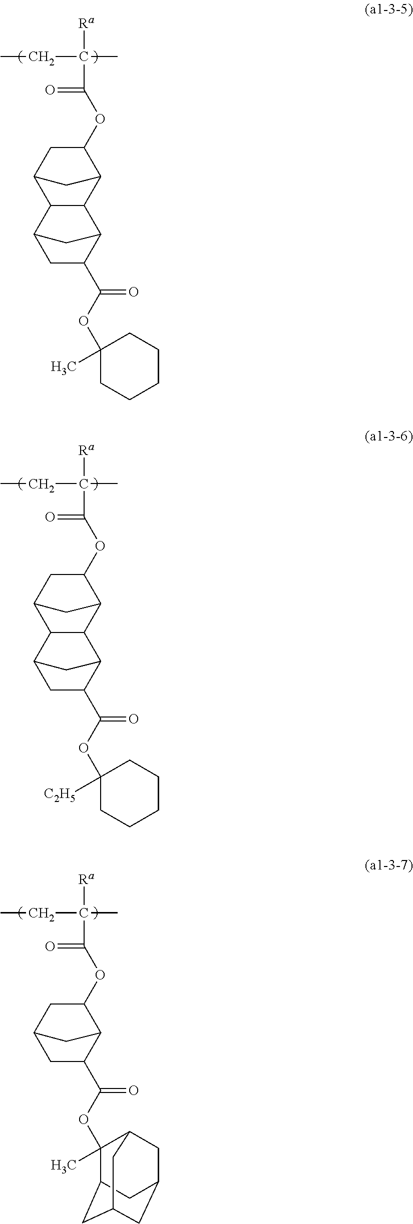

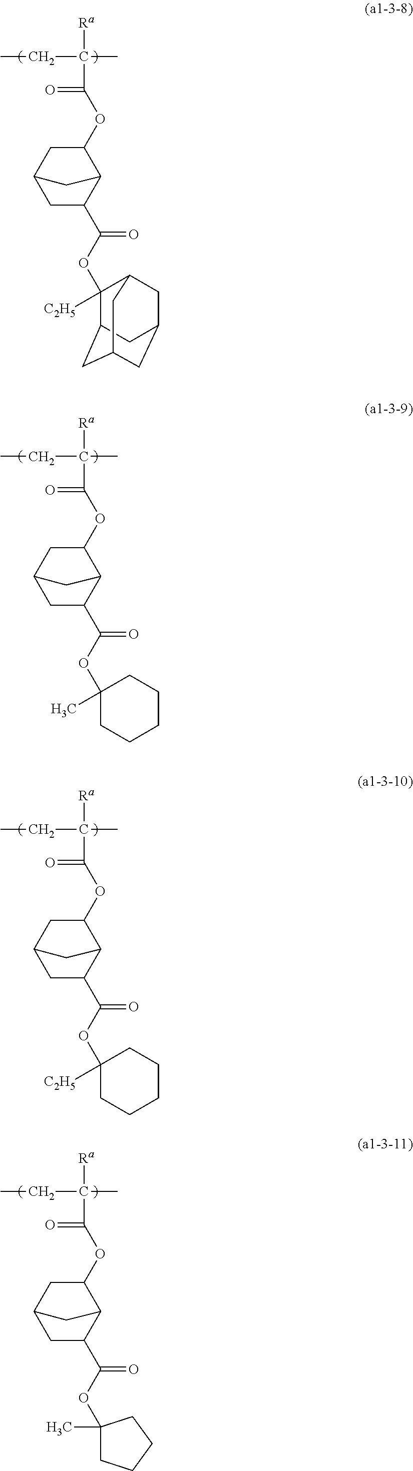

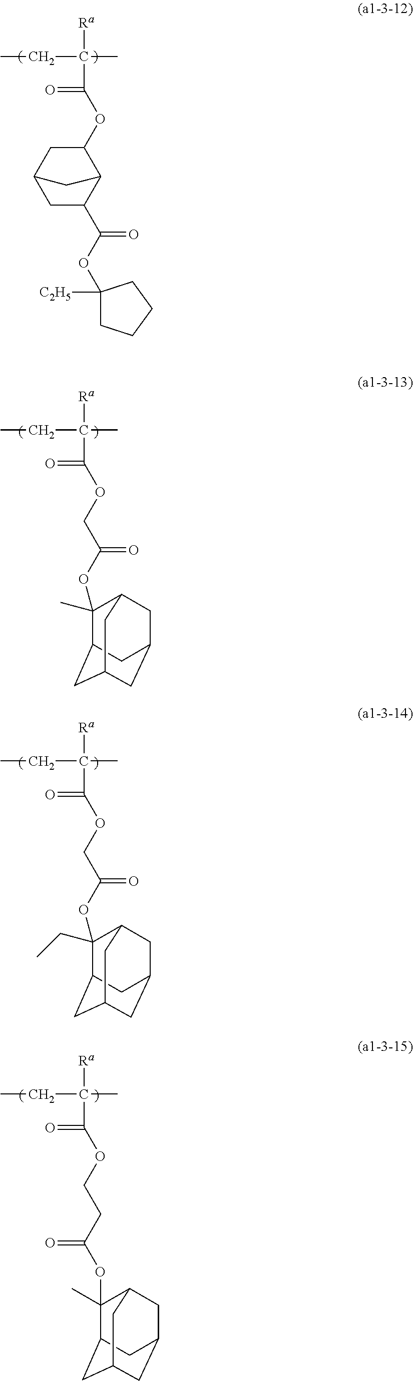

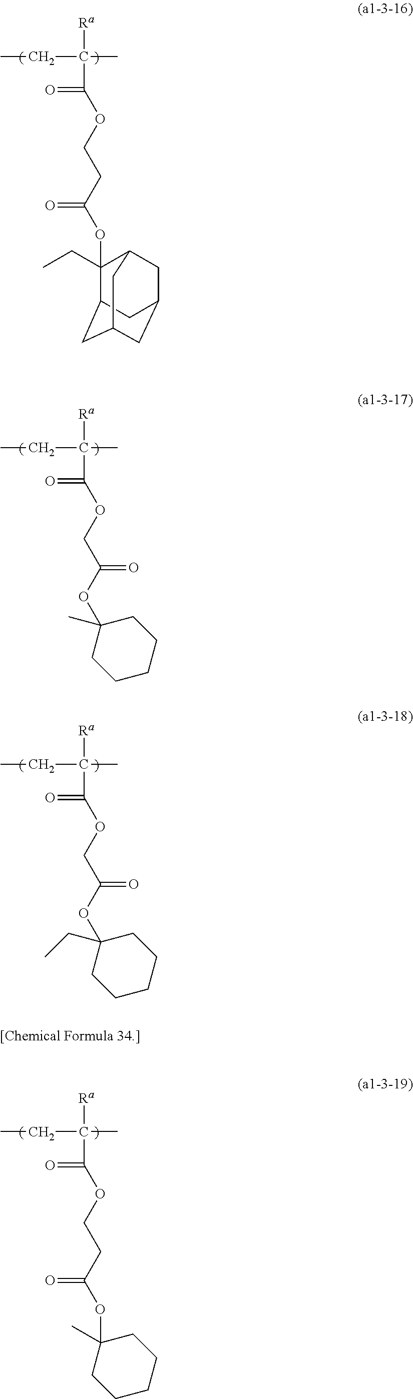

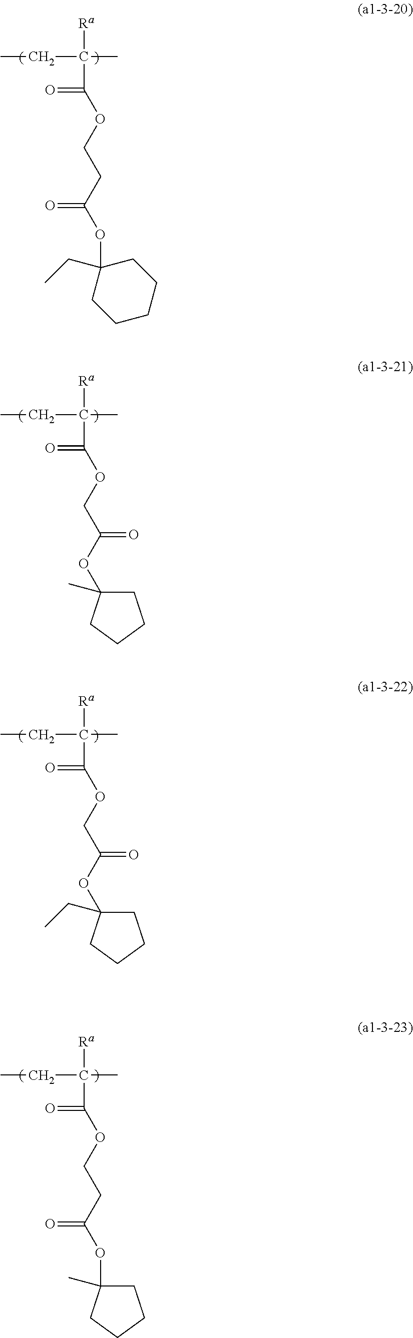

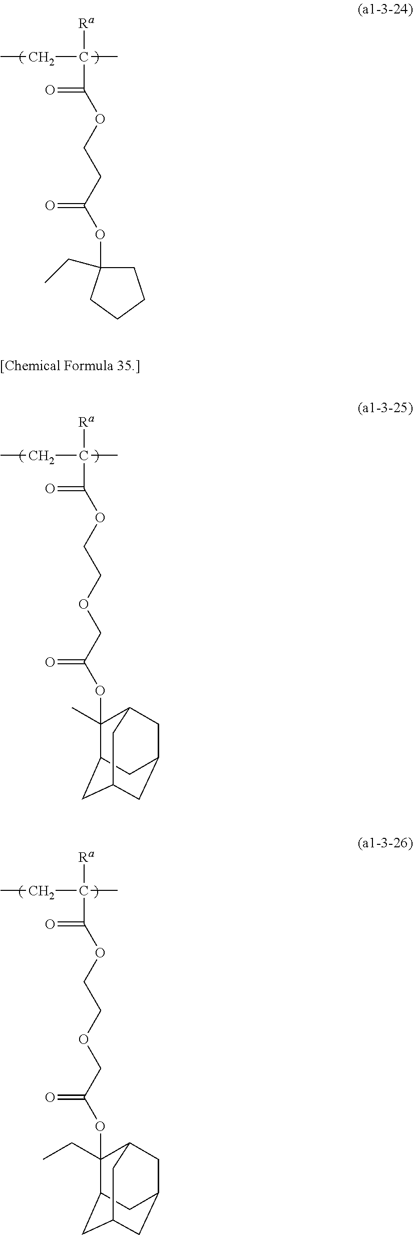

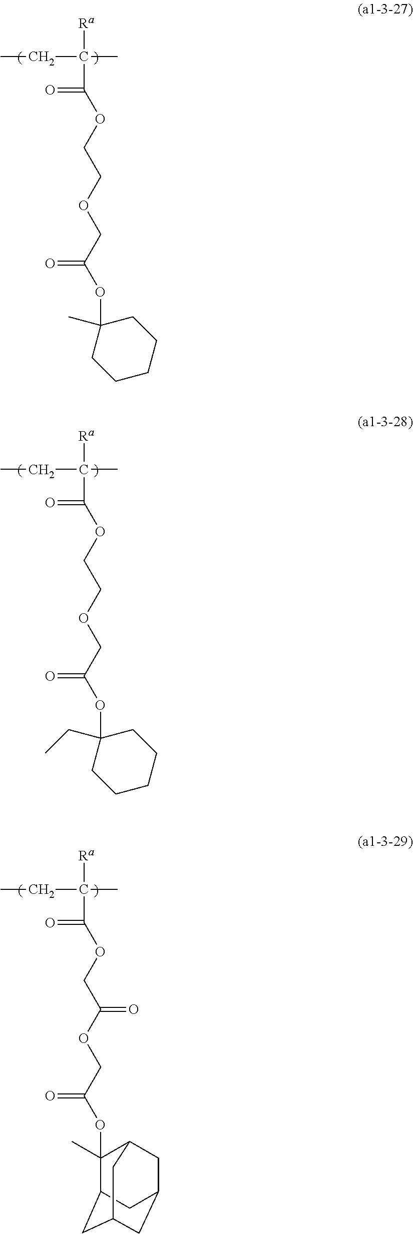

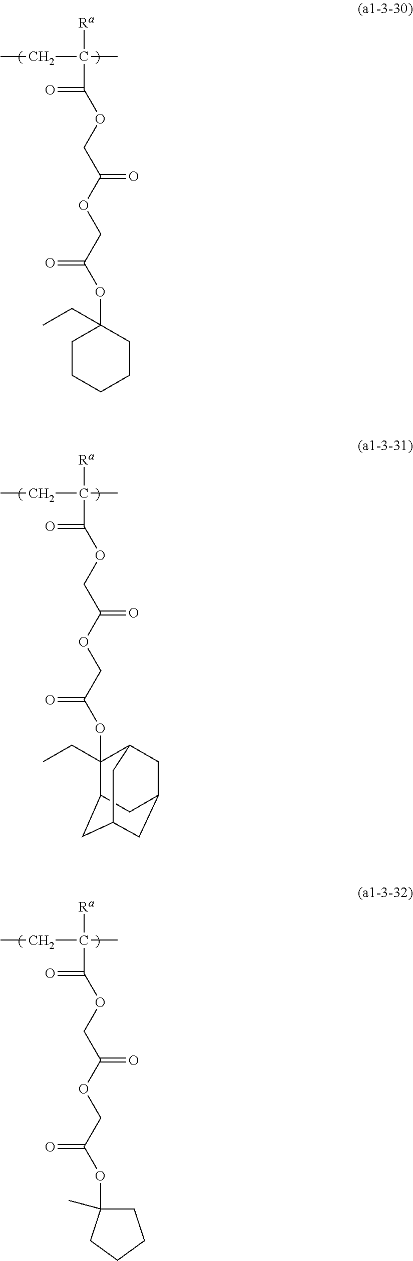

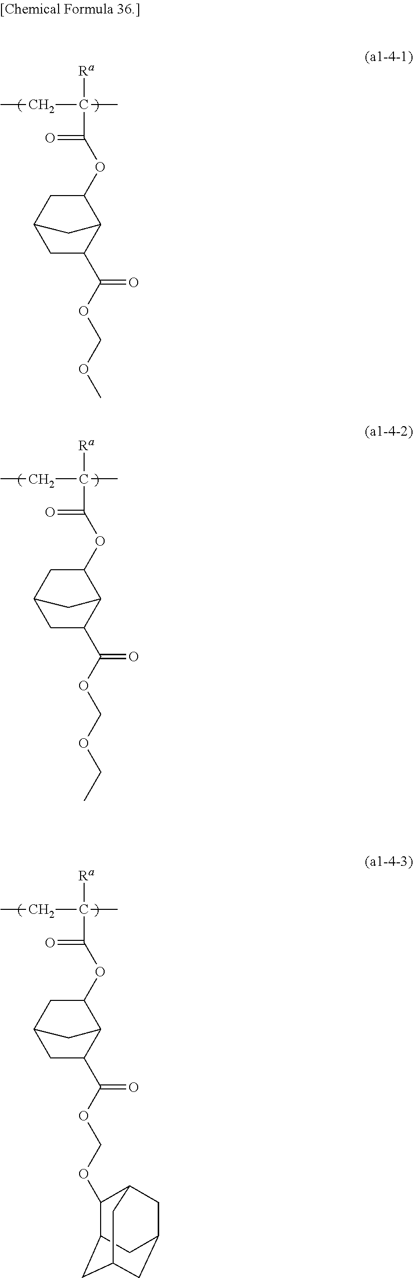

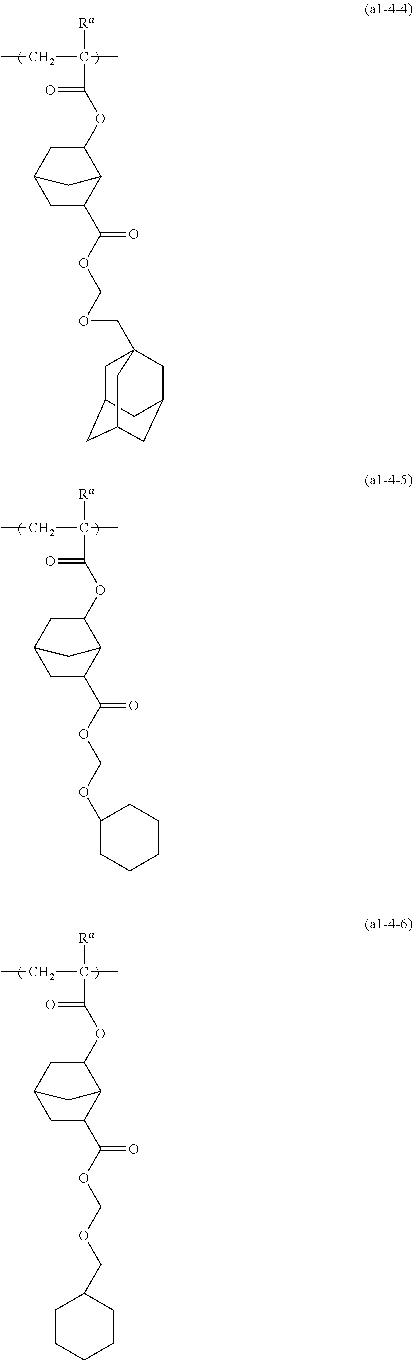

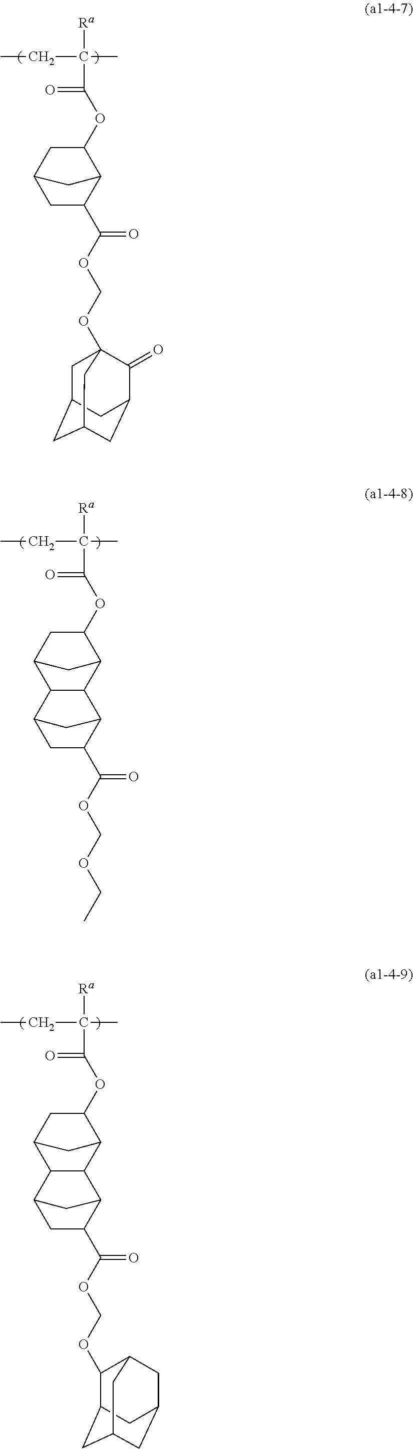

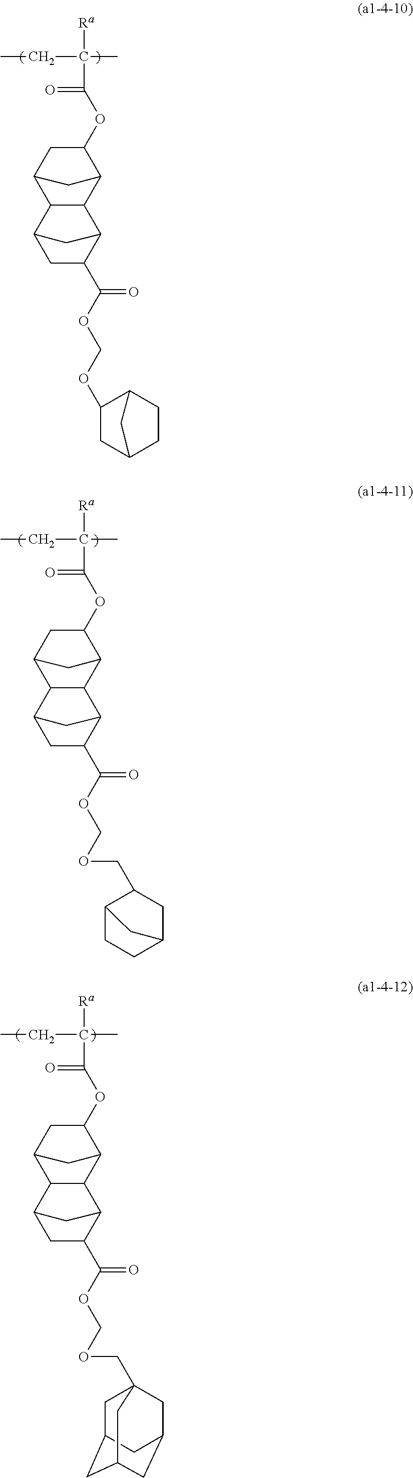

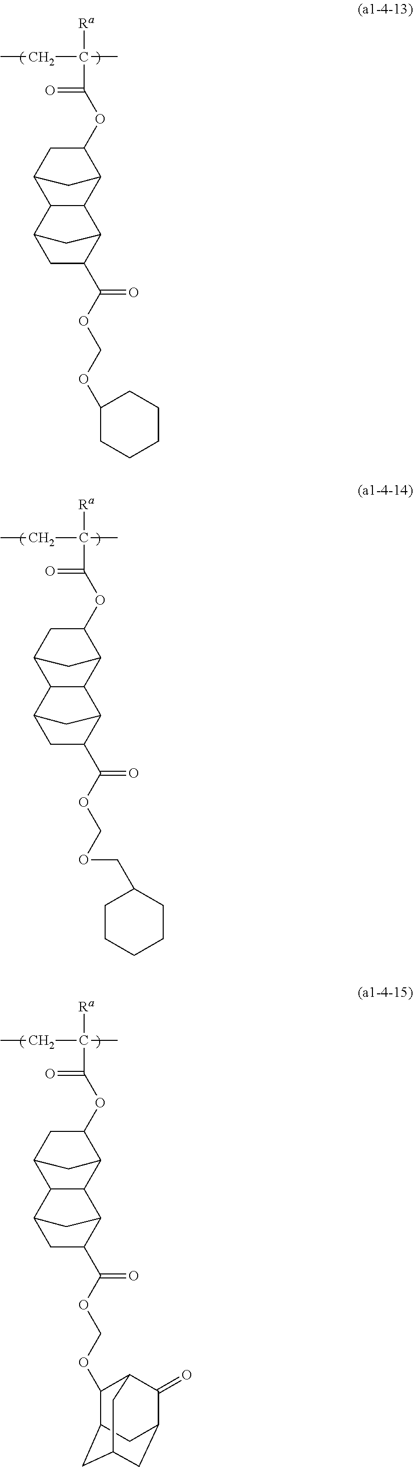

As the structural unit (a1), one type of structural unit may be used, or two or more types may be used in combination.

Among these, structural units represented by general formula (a1-1) or (a1-3) are preferable. More specifically, at least one structural unit selected from the group consisting of structural units represented by formulas (a1-1-1) to (a-1-1-4), (a1-1-20) to (a1-1-23) and (a1-3-25) to (a1-3-28) is more preferable.

Further, as the structural unit (a1), structural units represented by general formula (a1-1-01) shown below which includes the structural units represented by formulas (a1-1-1) to (a1-1-3), structural units represented by general formula (a1-1-02) shown below which includes the structural units represented by formulas (a1-1-16), (a1-1-17) and (a1-1-20) to (a1-1-23), structural units represented by general formula (a1-3-01) shown below which include the structural units represented by formulas (a1-3-25) and (a1-3-26), structural units represented by general formula (a1-3-02) shown below which include the structural units represented by formulas (a1-3-27) and (a1-3-28), and structural units represented by general formula (a1-3-03) shown below which include the structural units represented by formulas (a1-3-29) and (a1-3-30) are also preferable.

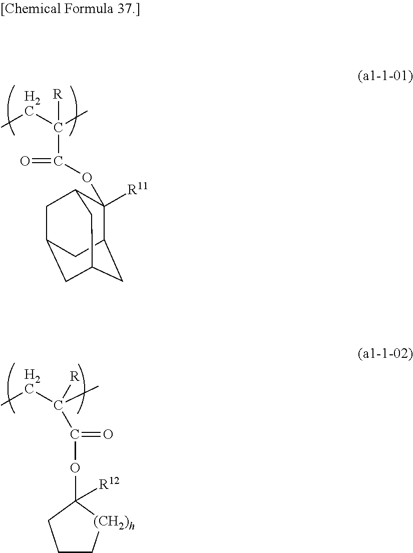

In general formula (a1-1-01), R represents a hydrogen atom, an alkyl group of 1 to 5 carbon atoms or a halogenated alkyl group of 1 to 5 carbon atoms; and R¹¹ represents an alkyl group of 1 to 5 carbon atoms. In general formula (a1-1-02), R is the same as defined above; R¹² represents an alkyl group of 1 to 5 carbon atoms; and h represents an integer of 1 to 6.

In general formula (a1-1-01), R is the same as defined above.

The alkyl group of 1 to 5 carbon atoms for R¹¹ is the same as defined for the alkyl group of 1 to 5 carbon atoms for R, and a methyl group, an ethyl group or an isopropyl group is preferable.

In general formula (a1-1-02), R is the same as defined above.

The alkyl group of 1 to 5 carbon atoms for R¹² is the same as defined for the alkyl group of 1 to 5 carbon atoms for R, and a methyl group, an ethyl group or an isopropyl group is preferable. h is preferably 1 or 2, and most preferably 2.

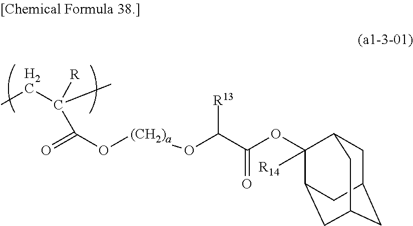

In the formula, R represents a hydrogen atom, an alkyl group of 1 to 5 carbon atoms or a halogenated alkyl group of 1 to 5 carbon atoms; R¹⁴ represents an alkyl group of 1 to 5 carbon atoms; R¹³ represents a hydrogen atom or a methyl group; and a represents an integer of 1 to 10.

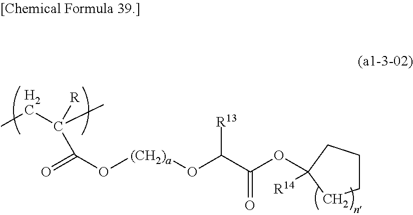

In the formula, R represents a hydrogen atom, an alkyl group of 1 to 5 carbon atoms or a halogenated alkyl group of 1 to 5 carbon atoms; R¹⁴ represents an alkyl group of 1 to 5 carbon atoms; R¹³ represents a hydrogen atom or a methyl group; a represents an integer of 1 to 10; and n′ represents an integer of 1 to 6.

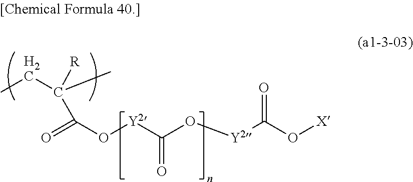

In the formula, R is as defined above; each of Y²′ and Y²″ independently represents a divalent linking group; X′ represents an acid dissociable, dissolution inhibiting group; and n represents an integer of 0 to 3.

In general formulas (a1-3-01) to (a1-3-03), R is the same as defined above.

R¹³ is preferably a hydrogen atom.

n′ is preferably 1 or 2, and most preferably 2.

a is preferably an integer of 1 to 8, more preferably an integer of 2 to 5, and most preferably 2.

As the divalent linking group for Y²′ and Y²″, the same groups as those described above for Y² in general formula (a1-3) can be used.

As Y²′, a divalent hydrocarbon group which may have a substituent is preferable, a linear aliphatic hydrocarbon group is more preferable, and a linear alkylene group is still more preferable. Among linear alkylene groups, a linear alkylene group of 1 to 5 carbon atoms is preferable, and a methylene group or an ethylene group is particularly desirable.

As Y²″, a divalent hydrocarbon group which may have a substituent is preferable, a linear aliphatic hydrocarbon group is more preferable, and a linear alkylene group is still more preferable. Among linear alkylene groups, a linear alkylene group of 1 to 5 carbon atoms is preferable, and a methylene group or an ethylene group is particularly desirable.

As the acid dissociable, dissolution inhibiting group for X′, the same groups as those described above can be used. X′ is preferably a tertiary alkyl ester-type acid dissociable, dissolution inhibiting group, more preferably the aforementioned group (i) which has a tertiary carbon atom on the ring structure of a monovalent aliphatic cyclic group. Among the aforementioned groups (i), a group represented by general formula (1-1) above is preferable.

n represents an integer of 0 to 3, preferably an integer of 0 to 2, more preferably 0 or 1, and most preferably 1.

In the component (A1), the amount of the structural unit (a1) based on the combined total of all structural units constituting the component (A1) is preferably 10 to 80 mol %, more preferably 20 to 70 mol %, and still more preferably 25 to 50 mol %. When the amount of the structural unit (a1) is at least as large as the lower limit of the above-mentioned range, a pattern can be easily formed using a positive resist composition prepared from the component (A1). On the other hand, when the amount of the structural unit (a1) is no more than the upper limit of the above-mentioned range, a good balance can be achieved with the other structural units.

Structural Unit (a2)

The structural unit (a2) is a structural unit derived from an acrylate ester containing a lactone-containing cyclic group.

The term “lactone-containing cyclic group” refers to a cyclic group including one ring containing a —O—C(O)— structure (lactone ring). The term “lactone ring” refers to a single ring containing a —O—C(O)— structure, and this ring is counted as the first ring. A lactone-containing cyclic group in which the only ring structure is the lactone ring is referred to as a monocyclic group, and groups containing other ring structures are described as polycyclic groups regardless of the structure of the other rings.

When the component (A1) is used for forming a resist film, the lactone-containing cyclic group of the structural unit (a2) is effective in improving the adhesion between the resist film and the substrate, and increasing the compatibility with the developing solution containing water.

As the structural unit (a2), there is no particular limitation, and an arbitrary structural unit may be used.

Specific examples of lactone-containing monocyclic groups include a group in which one hydrogen atom has been removed from a 4- to 6-membered lactone ring, such as a group in which one hydrogen atom has been removed from β-propionolactone, a group in which one hydrogen atom has been removed from γ-butyrolactone, and a group in which one hydrogen atom has been removed from δ-valerolactone. Further, specific examples of lactone-containing polycyclic groups include groups in which one hydrogen atom has been removed from a lactone ring-containing bicycloalkane, tricycloalkane or tetracycloalkane.

More specifically, examples of the structural unit (a2) include structural units represented by general formulas (a2-1) to (a2-5) shown below.

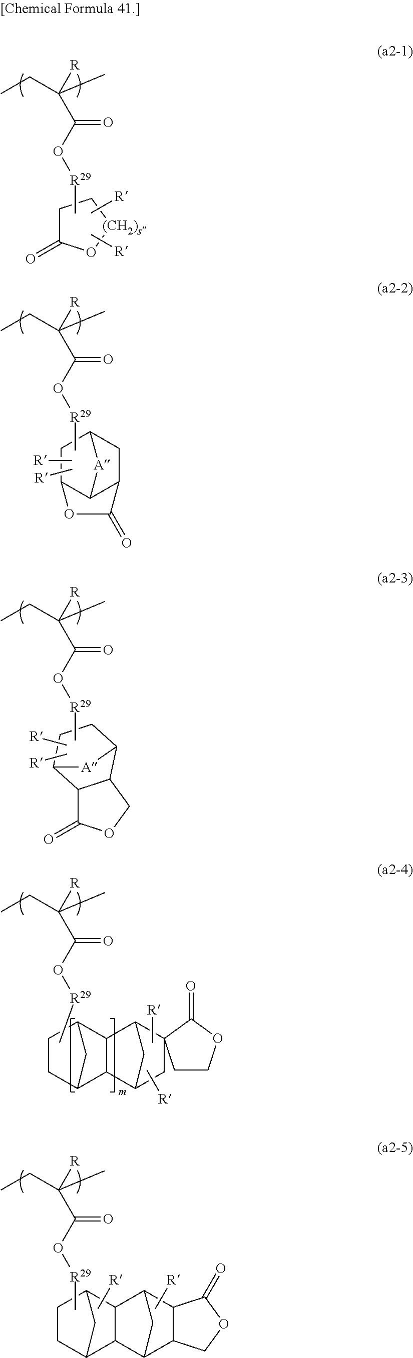

In the formulas, R represents a hydrogen atom, an alkyl group of 1 to 5 carbon atoms or a halogenated alkyl group of 1 to 5 carbon atoms; each R′ independently represents a hydrogen atom, an alkyl group of 1 to 5 carbon atoms, an alkoxy group of 1 to 5 carbon atoms or —COOR″, wherein R″ represents a hydrogen atom or an alkyl group; R²⁹ represents a single bond or a divalent linking group; s″ represents an integer of 0 to 2; A″ represents an oxygen atom, a sulfur atom or an alkylene group of 1 to 5 carbon atoms which may contain an oxygen atom or a sulfur atom; and m represents 0 or 1.

In general formulas (a2-1) to (a2-5), R is the same as defined for R in the structural unit (a1).

Examples of the alkyl group of 1 to 5 carbon atoms for R′ include a methyl group, an ethyl group, a propyl group, an n-butyl group and a tert-butyl group.

Examples of the alkoxy group of 1 to 5 carbon atoms for R′ include a methoxy group, an ethoxy group, an n-propoxy group, an iso-propoxy group, an n-butoxy group and a tert-butoxy group

In terms of industrial availability, R′ is preferably a hydrogen atom.

When R″ is a linear or branched alkyl group, it preferably has 1 to 10 carbon atoms, more preferably 1 to 5 carbon atoms.

When R″ is a cyclic alkyl group (cycloalkyl group), it preferably has 3 to 15 carbon atoms, more preferably 4 to 12 carbon atoms, and most preferably 5 to 10 carbon atoms. As examples of the cycloalkyl group, groups in which one or more hydrogen atoms have been removed from a monocycloalkane or a polycycloalkane such as a bicycloalkane, tricycloalkane or tetracycloalkane, which may or may not be substituted with a fluorine atom or a fluorinated alkyl group, may be used. Examples of such groups include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane or tetracyclododecane.

As A″, an alkylene group of 1 to 5 carbon atoms or —O— is preferable, more preferably an alkylene group of 1 to 5 carbon atoms, and most preferably a methylene group.

R²⁹ represents a single bond or a divalent linking group. Examples of divalent linking groups include the same divalent linking groups as those described above for Y² in general formula (a1-0-2). Among these, an alkylene group, an ester bond (—C(═O)—O—) or a combination thereof is preferable. The alkylene group as a divalent linking group for R²⁹ is preferably a linear or branched alkylene group. Specific examples include the same linear alkylene groups and branched alkylene groups as those described above for the aliphatic cyclic group A in Y².

s″ is preferably 1 or 2.

Specific examples of structural units represented by general formulas (a2-1) to (a2-5) are shown below. In the formulas shown below, R^{α} represents a hydrogen atom, a methyl group or a trifluoromethyl group.

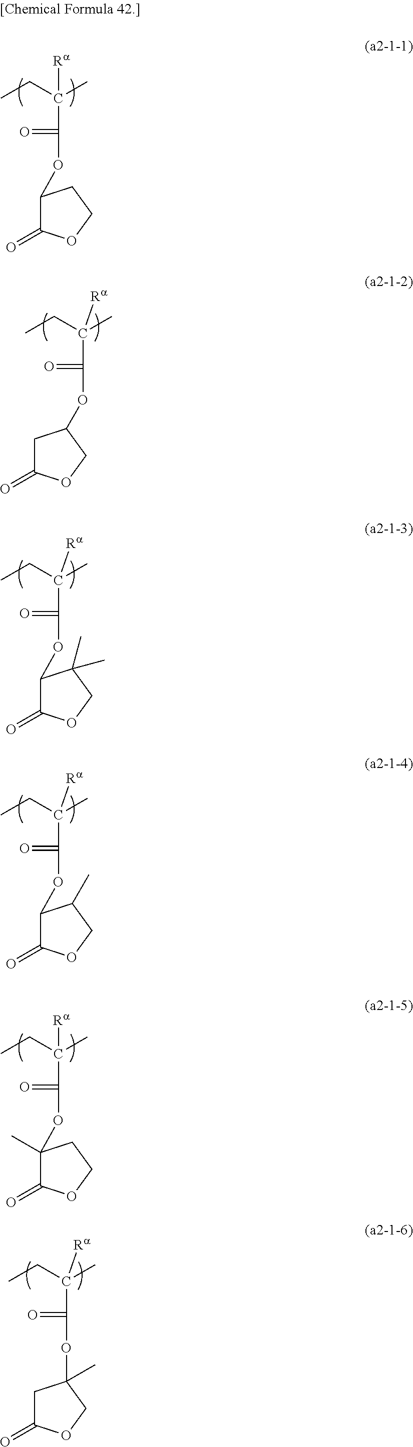

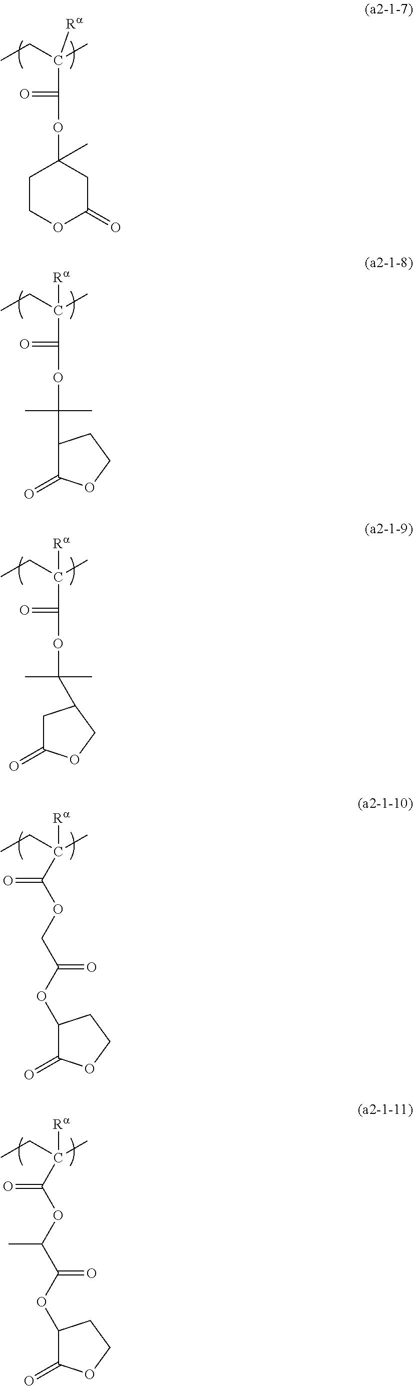

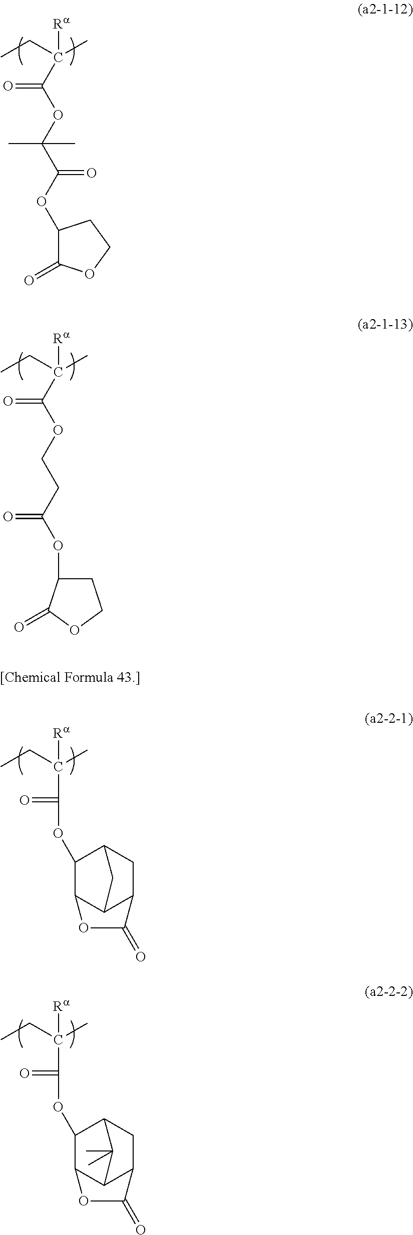

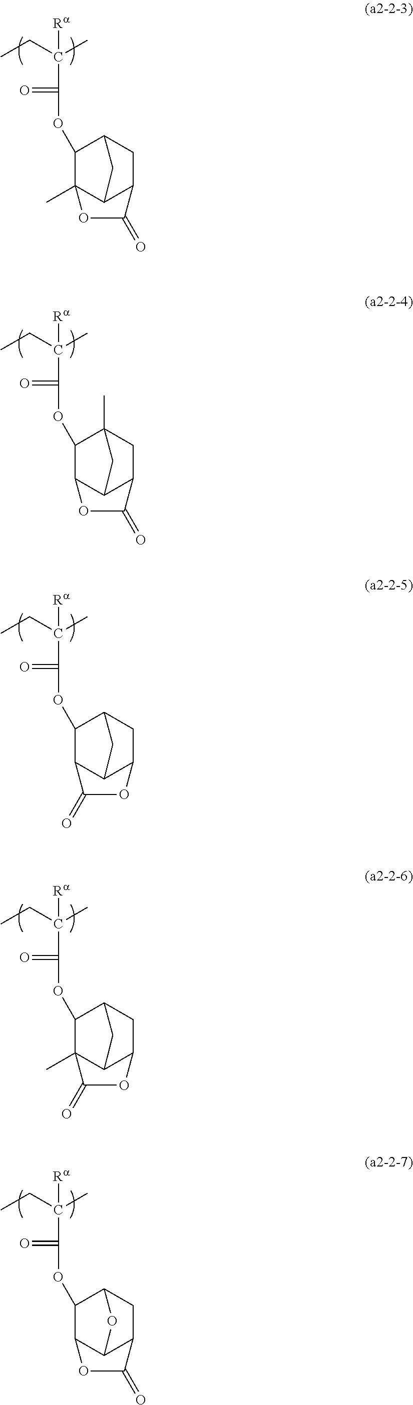

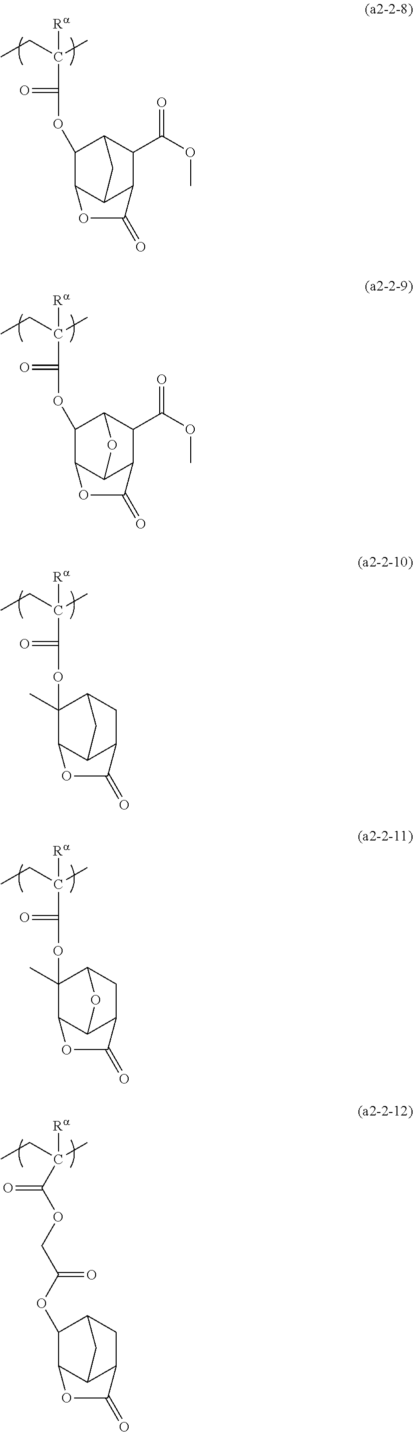

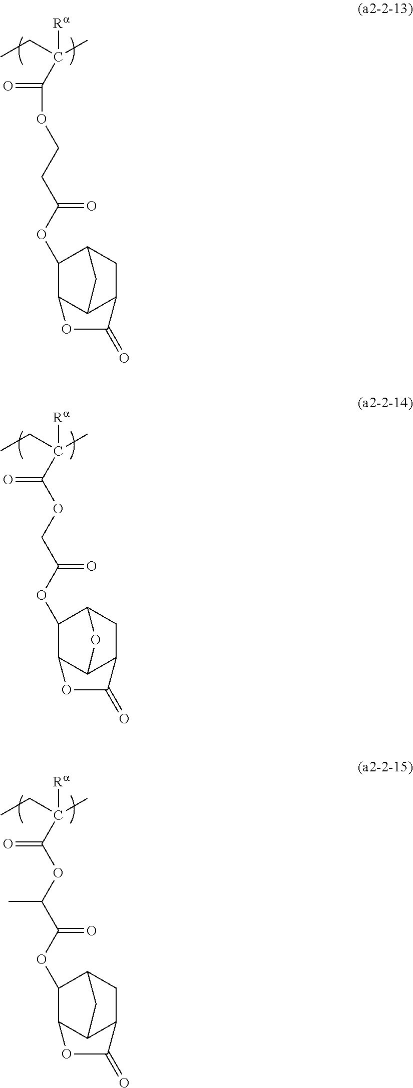

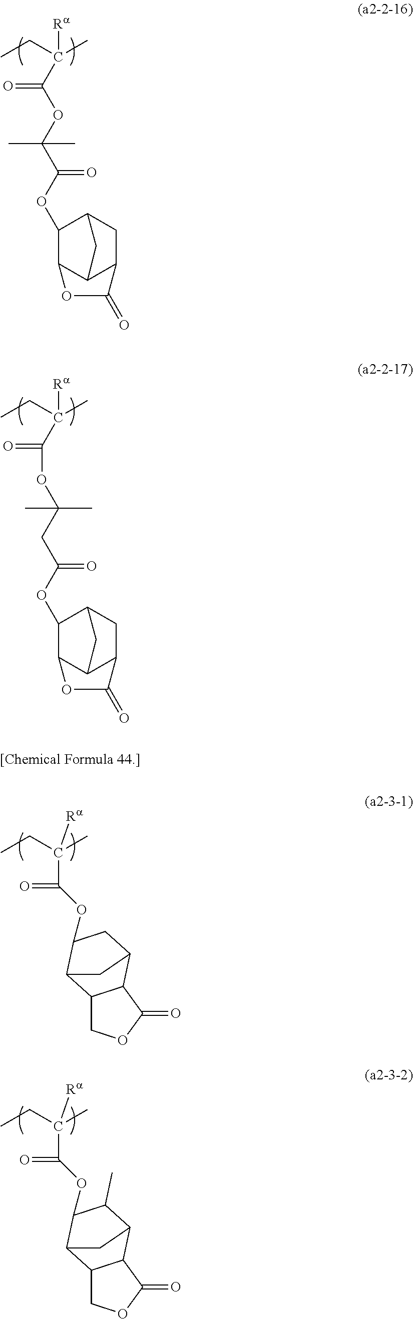

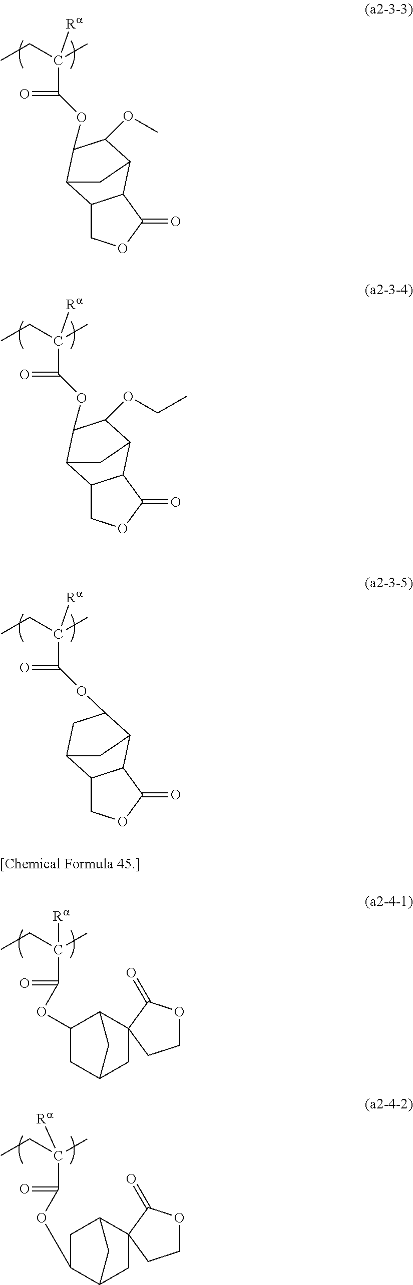

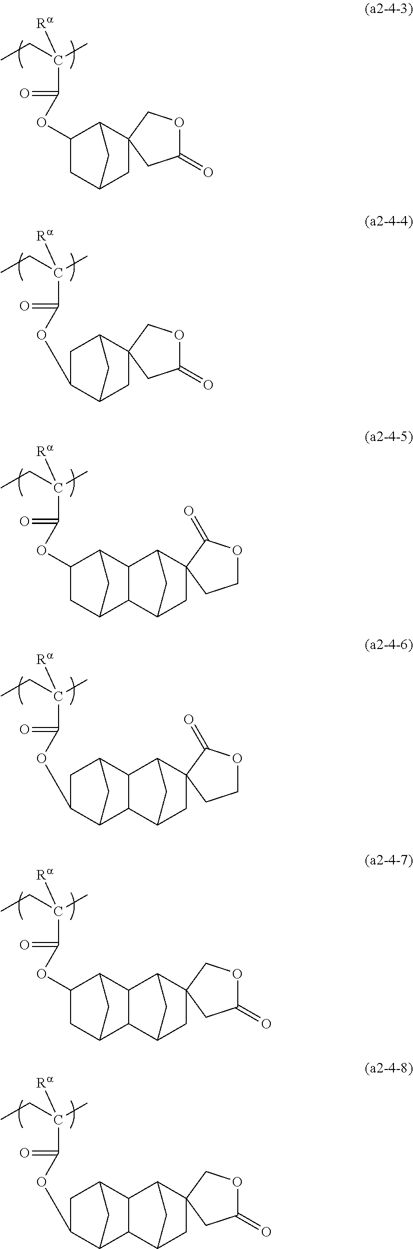

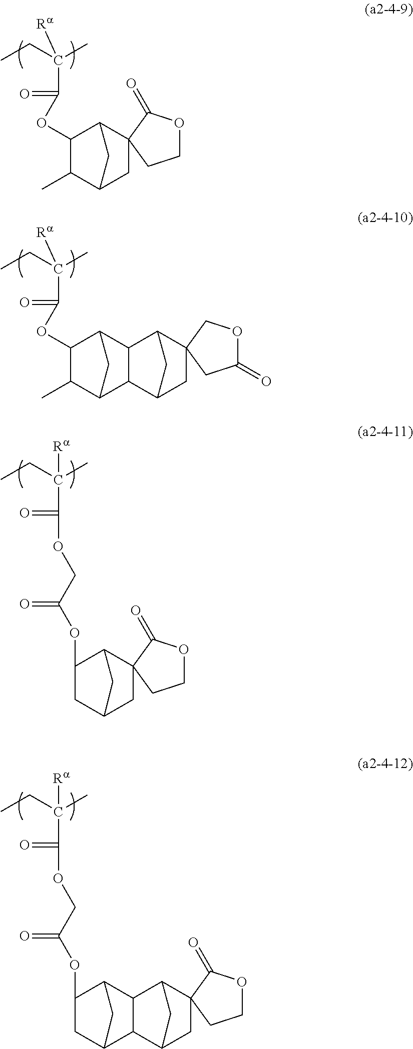

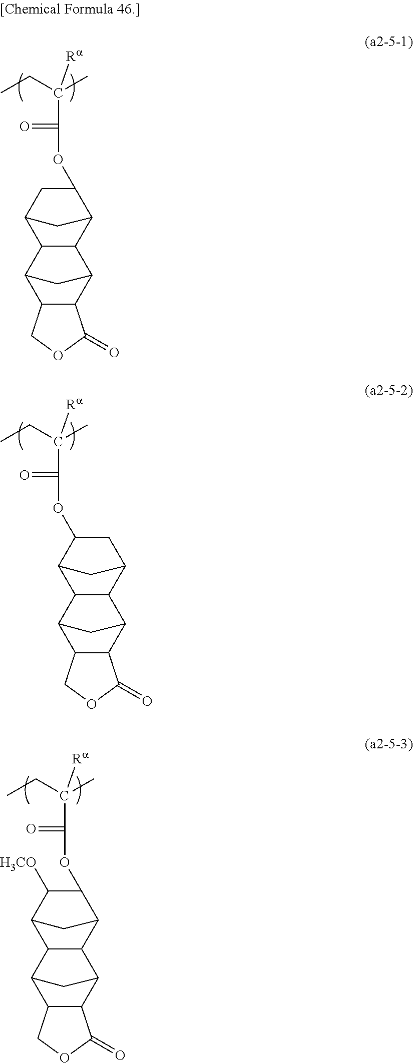

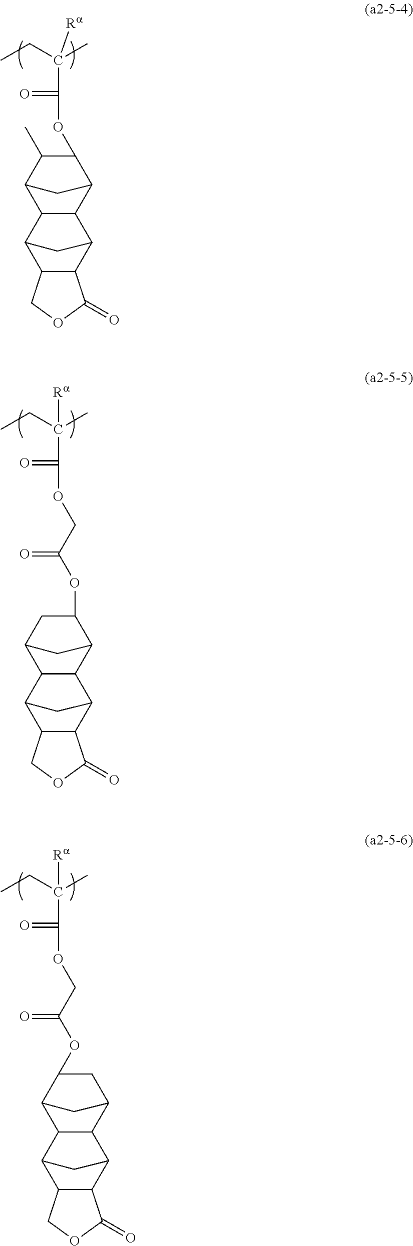

In the component (A1), as the structural unit (a2), one type of structural unit may be used, or two or more types may be used in combination.

As the structural unit (a2), at least one structural unit selected from the group consisting of formulas (a2-1) to (a2-5) is preferable, and at least one structural unit selected from the group consisting of formulas (a2-1) to (a2-3) is more preferable. Of these, it is preferable to use at least one structural unit selected from the group consisting of structural units represented by formulas (a2-1-1), (a2-2-1), (a2-2-7), (a2-3-1) and (a2-3-5).

In the component (A1), the amount of the structural unit (a2) based on the combined total of all structural units constituting the component (A1) is preferably 5 to 60 mol %, more preferably 10 to 50 mol %, and still more preferably 20 to 50 mol %. When the amount of the structural unit (a2) is at least as large as the lower limit of the above-mentioned range, the effect of using the structural unit (a2) can be satisfactorily achieved. On the other hand, when the amount of the structural unit (a2) is no more than the upper limit of the above-mentioned range, a good balance can be achieved with the other structural units.

Structural Unit (a3)

The structural unit (a3) is a structural unit derived from an acrylate ester containing a polar group-containing aliphatic hydrocarbon group.

When the component (A1) includes the structural unit (a3), the hydrophilicity of the component (A) is improved, and hence, the compatibility of the component (A) with the developing solution is improved. As a result, the alkali solubility of the exposed portions improves, which contributes to favorable improvements in the resolution.

Examples of the polar group include a hydroxyl group; cyano group, carboxyl group, or hydroxyalkyl group in which some of the hydrogen atoms of the alkyl group have been substituted with fluorine atoms, although a hydroxyl group is particularly desirable.

Examples of the aliphatic hydrocarbon group include linear or branched hydrocarbon groups (preferably alkylene groups) of 1 to 10 carbon atoms, and polycyclic aliphatic hydrocarbon groups (polycyclic groups). These polycyclic groups can be selected appropriately from the multitude of groups that have been proposed for the resins of resist compositions designed for use with ArF excimer lasers. The polycyclic group preferably has 7 to 30 carbon atoms.

Of the various possibilities, structural units derived from an acrylate ester that include an aliphatic polycyclic group that contains a hydroxyl group, cyano group, carboxyl group or a hydroxyalkyl group in which part of the hydrogen atoms of the alkyl group have been substituted with fluorine atoms are particularly desirable. Examples of the polycyclic group include groups in which two or more hydrogen atoms have been removed from a bicycloalkane, tricycloalkane, tetracycloalkane or the like. Specific examples include groups in which two or more hydrogen atoms have been removed from a polycycloalkane such as adamantane, norbornane, isobornane, tricyclodecane or tetracyclododecane. Of these polycyclic groups, groups in which two or more hydrogen atoms have been removed from adamantane, norbornane or tetracyclododecane are preferred industrially.

When the aliphatic hydrocarbon group within the polar group-containing aliphatic hydrocarbon group is a linear or branched hydrocarbon group of 1 to 10 carbon atoms, the structural unit (a3) is preferably a structural unit derived from a hydroxyethyl ester of acrylic acid. On the other hand, when the hydrocarbon group is a polycyclic group, structural units represented by formulas (a3-1), (a3-2) and (a3-3) shown below are preferable.

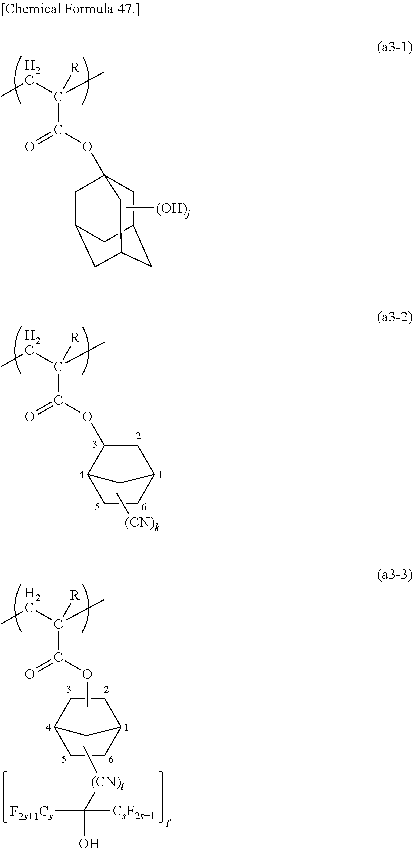

In the formulas, R is the same as defined above; j represents an integer of 1 to 3; k represents an integer of 1 to 3; t′ represents an integer of 1 to 3; 1 represents an integer of 1 to 5; and s represents an integer of 1 to 3.

In formula (a3-1), j is preferably 1 or 2, and more preferably 1. When j is 2, it is preferable that the hydroxyl groups be bonded to the 3rd and 5th positions of the adamantyl group. When j is 1, it is preferable that the hydroxyl group be bonded to the 3rd position of the adamantyl group.

In formula (a3-2), k is preferably 1. The cyano group is preferably bonded to the 5th or 6th position of the norbornyl group.

In formula (a3-3), t′ is preferably 1. l is preferably 1. s is preferably 1. Further, in formula (a3-3), it is preferable that a 2-norbornyl group or 3-norbornyl group be bonded to the terminal of the carboxy group of the acrylic acid. The fluorinated alkyl alcohol is preferably bonded to the 5th or 6th position of the norbornyl group.

In the component (A1), as the structural unit (a3), one type of structural unit may be used, or two or more types may be used in combination.

In the component (A1), the amount of the structural unit (a3) based on the combined total of all structural units constituting the component (A1) is preferably 5 to 50 mol %, more preferably 5 to 40 mol %, and still more preferably 5 to 25 mol %. When the amount of the structural unit (a3) is at least as large as the lower limit of the above-mentioned range, the effect of using the structural unit (a3) can be satisfactorily achieved. On the other hand, when the amount of the structural unit (a3) is no more than the upper limit of the above-mentioned range, a good balance can be achieved with the other structural units.

Structural Unit (a4)

The component (A1) may also have a structural unit (a4) which is other than the above-mentioned structural units (a1) to (a3), as long as the effects of the present invention are not impaired.

As the structural unit (a4), any other structural unit which cannot be classified as one of the above structural units (a1) to (a3) can be used without any particular limitation, and any of the multitude of conventional structural units used within resist resins for ArF excimer lasers or KrF excimer lasers (and particularly for ArF excimer lasers) can be used.

As the structural unit (a4), a structural unit which contains a non-acid-dissociable aliphatic polycyclic group, and is also derived from an acrylate ester is preferable. Examples of this polycyclic group include the same groups as those described above in relation to the aforementioned structural unit (a1), and any of the multitude of conventional polycyclic groups used within the resin component of resist compositions for ArF excimer lasers or KrF excimer lasers (and particularly for ArF excimer lasers) can be used.

In consideration of industrial availability and the like, at least one polycyclic group selected from amongst a tricyclodecyl group, adamantyl group, tetracyclododecyl group, isobornyl group, and norbornyl group is particularly desirable. These polycyclic groups may be substituted with a linear or branched alkyl group of 1 to 5 carbon atoms.

Specific examples of the structural unit (a4) include units with structures represented by general formulas (a4-1) to (a4-5) shown below.

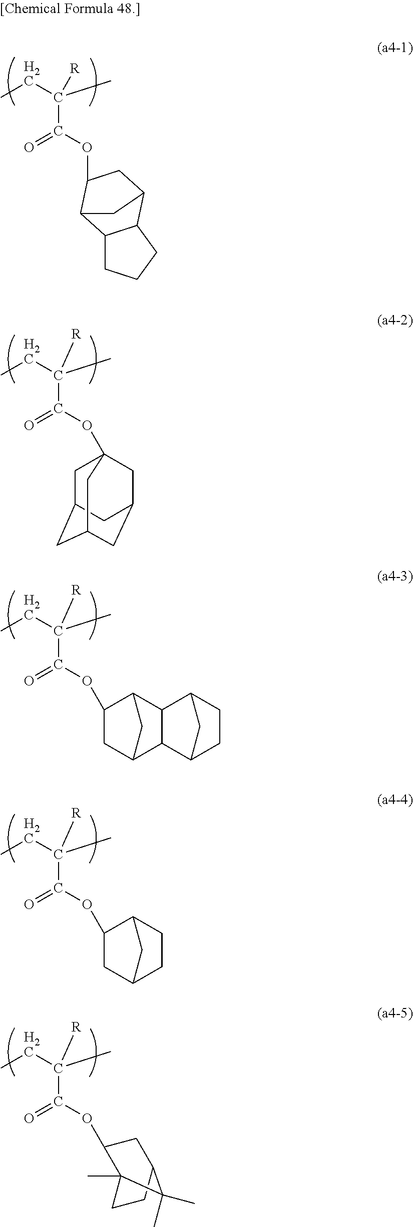

In the formulas, R is the same as defined above.

When the structural unit (a4) is included in the component (A1), the amount of the structural unit (a4) based on the combined total of all the structural units that constitute the component (A1) is preferably within the range from 1 to 30 mol %, and more preferably from 10 to 20 mol %.

In the present invention, the component (A1) is a copolymer having the structural units (a1), (a2) and (a3). Examples of such a copolymer include a copolymer consisting of the structural units (a1) and (a2) and (a3), and a copolymer consisting of the structural units (a1), (a2), (a3) and (a4).

In the component (A), as the component (A1), one type of resin may be used, or two or more types of resins may be used in combination.

In the present invention, as the component (A1), a polymeric compound that includes a combination of structural units such as that shown below is particularly desirable.

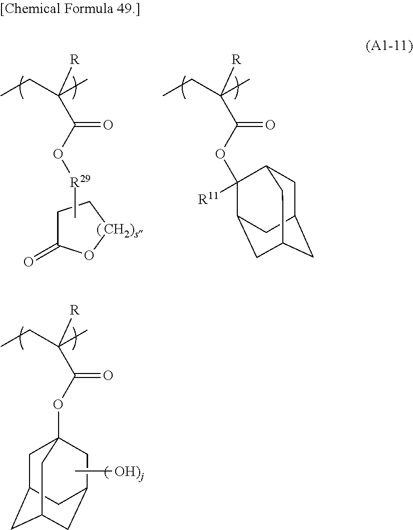

In the formula, R, R¹¹, R²⁹, s″ and j are the same as defined above, and the plurality of R may be the same or different from each other.

The weight average molecular weight (Mw) (the polystyrene equivalent value determined by gel permeation chromatography) of the component (A1) is not particularly limited, but is preferably 1,000 to 50,000, more preferably 1,500 to 30,000, and most preferably 2,500 to 20,000. When the weight average molecular weight is no more than the upper limit of the above-mentioned range, the resist composition exhibits a satisfactory solubility in a resist solvent. On the other hand, when the weight average molecular weight is at least as large as the lower limit of the above-mentioned range, dry etching resistance and the cross-sectional shape of the resist pattern becomes satisfactory.

Further, the dispersity (Mw/Mn) is preferably 1.0 to 5.0, more preferably 1.0 to 3.0, and most preferably 1.2 to 2.5. Here, Mn is the number average molecular weight.

In the positive resist composition of the present invention, the amount of the component (A1) can be appropriately adjusted depending on the thickness of the resist film to be formed, and the like.

<Component (B)>

In the resist composition of the present invention, the component (B) contains an acid generator (B1) including a compound represented by the aforementioned general formula (b1) (hereafter, this acid generator (B1) is referred to as “component (B1)”).

In formula (b1), R¹, R², x and n are the same as defined for the compound according to the third aspect of the present invention.

In formula (b1), X⁻ is the same as defined for the compound according to the third aspect of the present invention, and an anion represented by general formula (1-1-12-1) shown below is preferable.

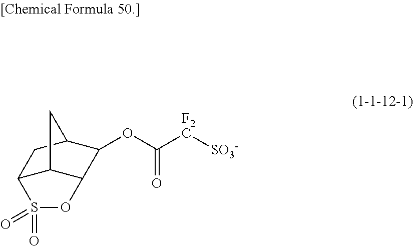

As the component (B), one type of compound may be used, or two or more types of compounds may be mixed together for use.

In the resist composition of the present invention, the amount of the component (B1) based on the entire component (B) is preferably 5% by weight or more, still more preferably 60% by weight or more, and may be even 100% by weight.

When the amount of the component (B1) is at least as large as the lower limit of the above-mentioned range, the shape of the resist pattern becomes excellent. In particular, when the resist composition is used for immersion exposure or for forming an upper-layer resist film, the lithography properties are improved. Further, in the resist composition of the present invention, the amount of the component (B1), relative to 100 parts by weight of the component (A) is preferably 1 to 70 parts by weight, still more preferably 3 to 60 parts by weight, and most preferably 5 to 50 parts by weight.

When the amount of the component (B1) is at least as large as the lower limit of the above-mentioned range, particularly when the resist composition is used for immersion exposure or for forming an upper-layer resist film, the lithography properties are improved. On the other hand, when the amount of the component (B1) is no more than the upper limit of the above-mentioned range, the storage stability becomes excellent.

In the component (B), an acid generator (B2) other than the component (B1) (hereafter, referred to as component (B2)) may be used in combination with the component (B1).

As the component (B2), there is no particular limitation as long as it is other than the component (B1), and any of the known acid generators used in conventional chemically amplified resist compositions can be used.

Examples of these acid generators are numerous, and include onium salt acid generators such as iodonium salts and sulfonium salts; oxime sulfonate acid generators; diazomethane acid generators such as bisalkyl or bisaryl sulfonyl diazomethanes and poly(bis-sulfonyl)diazomethanes; nitrobenzylsulfonate acid generators; iminosulfonate acid generators; and disulfone acid generators.

As an onium salt acid generator, a compound represented by general formula (b-1) or (b-2) shown below can be used.

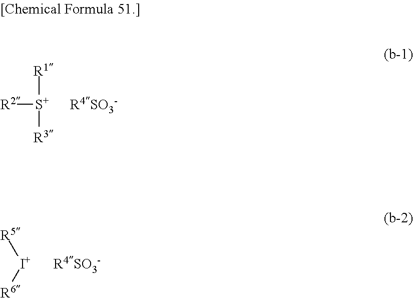

In the formulas above, R¹″ to R³″, R⁵″ and R⁶″ each independently represent an aryl group or alkyl group which may have a substituent, wherein two of R¹″ to R³″ may be bonded to each other to form a ring with the sulfur atom; and R⁴″ represents an alkyl group, a halogenated alkyl group, an aryl group or an alkenyl group which may have a substituent, provided that at least one of R¹″ to R³″ represents an aryl group, and at least one of R⁵″ and R⁶″ represents an aryl group.

In formula (b-1), R¹″ to R³″ each independently represents an aryl group which may have a substituent or an alkyl group which may have a substituent. In formula (b-1), two of R¹″ to R³″ may be bonded to each other to form a ring with the sulfur atom.

Further, among R¹″ to R³″, at least one group represents an aryl group. Among R¹″ to R³″, two or more groups are preferably aryl groups, and it is particularly desirable that all of R¹″ to R³″ are aryl groups.

The aryl group of R¹″ to R³″ is not particularly limited, and includes, for example, an aryl group of 6 to 20 carbon atoms. The aryl group is preferably an aryl group having 6 to 10 carbon atoms because it can be synthesized at a low cost. Specific examples thereof include a phenyl group and a naphthyl group.

The aryl group may have a substituent. The expression “has a substituent” means that part or all of the hydrogen atoms within the aryl group has been substituted with a substituent. Examples of the substituent include an alkyl group, an alkoxy group, a halogen atom, a hydroxyl group, an alkoxyalkyloxy group, —O—R⁵⁰—CO—O—R⁵¹ (in the formula, R⁵⁰ represents an alkylene group, and R⁵¹ represents an acid dissociable group).

The alkyl group, with which hydrogen atoms of the aryl group may be substituted, is preferably an alkyl group having 1 to 5 carbon atoms, and most preferably a methyl group, an ethyl group, a propyl group, an n-butyl group, or a tert-butyl group.

The alkoxy group, with which hydrogen atoms of the aryl group may be substituted, is preferably an alkoxy group having 1 to 5 carbon atoms, more preferably a methoxy group, an ethoxy group, an n-propoxy group, an iso-propoxy group, an n-butoxy group or a tert-butoxy group, and most preferably a methoxy group or an ethoxy group.

The halogen atom, with which hydrogen atoms of the aryl group may be substituted, is preferably a fluorine atom.

Examples of the alkoxyalkyloxy group which substitutes the hydrogen atoms within the aryl group include —O—C(R⁴⁷)(R⁴⁸)—O—R⁴⁹ (in the formula, each of R⁴⁷ and R⁴⁸ independently represents a hydrogen atom or a linear or branched alkyl group, and R⁴⁹ represents an alkyl group, wherein R⁴⁸ and R⁴⁹ may be mutually bonded to form a ring structure, provided that at least one of R⁴⁷ and R⁴⁸ represents a hydrogen atom.

The alkyl group for R⁴⁷ and R⁴⁸ preferably has 1 to 5 carbon atoms. As the alkyl group, an ethyl group or a methyl group is preferable, and a methyl group is most preferable.

Further, it is preferable that at least one of R⁴⁷ and R⁴⁸ represent a hydrogen atom, and the other represent a hydrogen atom or a methyl group. It is particularly desirable that both of R⁴⁷ and R⁴⁸ represent a hydrogen atom.

The alkyl group for R⁴⁹ preferably has 1 to 15 carbon atoms, and may be linear, branched or cyclic.

The linear or branched alkyl group for R⁴⁹ preferably has 1 to 5 carbon atoms. Examples thereof include a methyl group, an ethyl group, a propyl group, an n-butyl group and a tert-butyl group.

The cyclic alkyl group for R⁴⁹ preferably has 4 to 15 carbon atoms, more preferably 4 to 12, and most preferably 5 to 10.

Specific examples thereof include groups in which one or more hydrogen atoms have been removed from a monocycloalkane or a polycycloalkane such as a bicycloalkane, tricycloalkane or tetracycloalkane, and which may or may not be substituted with an alkyl group of 1 to 5 carbon atoms, a fluorine atom or a fluorinated alkyl group. Examples of the monocycloalkane include cyclopentane and cyclohexane. Examples of polycycloalkanes include adamantane, norbornane, isobornane, tricyclodecane and tetracyclododecane. Among these, a group in which one or more hydrogen atoms have been removed from adamantane is preferable.

R⁴⁸ and R⁴⁹ may be mutually bonded to form a ring structure. In such a case, a cyclic group is formed by R⁴⁸, R⁴⁹, the oxygen atom having R⁴⁹ bonded thereto, and the carbon atom having the oxygen atom and R⁴⁸ bonded thereto. Such a cyclic group is preferably a 4- to 7-membered ring, and more preferably a 4- to 6-membered ring.

In the —O—R⁵⁰—CO—O—R⁵¹ group which may substitute the hydrogen atoms within the aryl group, the alkylene group for R⁵⁰ is preferably a linear or branched alkylene group of 1 to 5 carbon atoms. Examples of the alkylene group include a methylene group, an ethylene group, a trimethylene group, a tetramethylene group and a 1,1-dimethylethylene group.

The acid dissociable group for R⁵¹ is not particularly limited as long as it is an organic group that is dissociable by the action of an acid (generated from the component (B) upon exposure), and examples thereof include the same acid dissociable, dissolution inhibiting groups as those described above for the aforementioned structural unit (a1). However, unlike the aforementioned acid dissociable, dissolution inhibiting group, the acid dissociable group is not necessarily required to exhibit the dissolution inhibiting effect in an alkali developing solution.

Specific examples of the acid dissociable group include a tertiary alkyl ester-type acid dissociable group such as a cyclic or chain-like tertiary alkyl group; and an acetal-type acid dissociable group such as an alkoxyalkyl group. Among these, a tertiary alkyl ester-type acid dissociable group is preferable.

Specific examples of the tertiary alkyl ester-type acid dissociable group include a 2-methyl-2-adamantyl group, a 2-ethyl-2-adamantyl group, a 1-methyl-1-cyclopentyl group, a 1-ethyl-1-cyclopentyl group, a 1-methyl-1-cyclohexyl group, a 1-ethyl-1-cyclohexyl group, a 1-(1-adamantyl)-1-methylethyl group, a 1-(1-adamantyl)-1-methylpropyl group, a 1-(1-adamantyl)-1-methylbutyl group, a 1-(1-adamantyl)-1-methylpentyl group, a 1-(1-cyclopentyl)-1-methylethyl group, a 1-(1-cyclopentyl)-1-methylpropyl group, a 1-(1-cyclopentyl)-1-methylbutyl group, a 1-(1-cyclopentyl)-1-methylpentyl group, a 1-(1-cyclohexyl)-1-methylethyl group, a 1-(1-cyclohexyl)-1-methylpropyl group, a 1-(1-cyclohexyl)-1-methylbutyl group, a 1-(1-cyclohexyl)-1-methylpentyl group, a tert-butyl group, a tert-pentyl group and a tert-hexyl group.

The alkyl group for R¹″ to R³″ is not particularly limited and includes, for example, a linear, branched or cyclic alkyl group having 1 to 10 carbon atoms. In terms of achieving excellent resolution, the alkyl group preferably has 1 to 5 carbon atoms. Specific examples thereof include a methyl group, an ethyl group, an n-propyl group, an isopropyl group, an n-butyl group, an isobutyl group, an n-pentyl group, a cyclopentyl group, a hexyl group, a cyclohexyl group, a nonyl group, and a decyl group, and a methyl group is most preferable because it is excellent in resolution and can be synthesized at a low cost.

The alkyl group may have a substituent. The expression “has a substituent” means that part or all of the hydrogen atoms within the alkyl group has been substituted with a substituent. Examples of the substituent include the same groups as those described above for the substituent of the aforementioned aryl group.

When two of R¹″ to R³″ in formula (b-1) are bonded to each other to form a ring with the sulfur atom, it is preferable that the two of R¹″ to R³″ form a 3 to 10-membered ring including the sulfur atom, and it is particularly desirable that the two of R¹″ to R³″ form a 5 to 7-membered ring including the sulfur atom.

When two of R¹″ to R³″ in formula (b-1) are bonded to each other to form a ring with the sulfur atom, the remaining one of R¹″ to R³″ is preferably an aryl group. As examples of the aryl group, the same as the above-mentioned aryl groups for R¹″ to R³″ can be given.

As preferable examples of the cation moiety for the compound represented by general formula (b-1), those represented by formulas (I-1-1) to (I-1-10) shown below can be given. Among these, a cation moiety having a triphenylmethane skeleton, such as a cation moiety represented by any one of formulas (I-1-1) to (I-1-8) shown below is particularly desirable.

In formulas (I-1-9) and (I-1-10), each of R⁹ and R¹⁰ independently represents a phenyl group or naphthyl group which may have a substituent, an alkyl group of 1 to 5 carbon atoms, an alkoxy group or a hydroxy group.

u is an integer of 1 to 3, and most preferably 1 or 2.

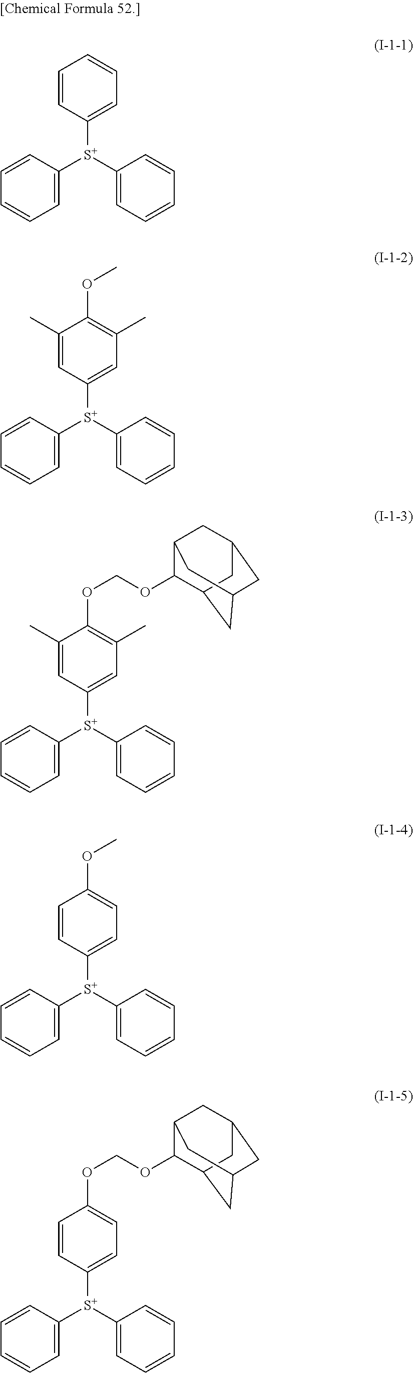

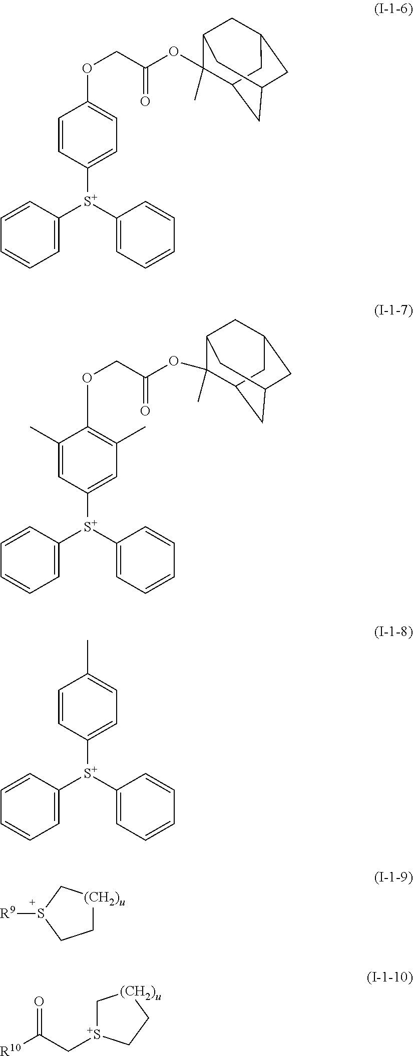

In formula (b-2), R⁵″ and R⁶″ each independently represent an aryl group or alkyl group. At least one of R⁵″ and R⁶″ represents an aryl group. It is preferable that both of R⁵″ and R⁶″ represent an aryl group.

As the aryl group for R⁵″ and R⁶″, the same as the aryl groups for R¹″ to R³″ can be used.

As the alkyl group for R⁵″ and R⁶″, the same as the alkyl groups for R¹″ to R³″ can be used.

It is particularly desirable that both of R⁵″ and R⁶″ represents a phenyl group.

As R⁴″ in formula (b-2), the same groups as those mentioned above for R⁴″ in formula (b-1) can be used.

Specific examples of suitable onium salt acid generators represented by formula (b-1) or (b-2) include diphenyliodonium trifluoromethanesulfonate or nonafluorobutanesulfonate; bis(4-tert-butylphenyl)iodonium trifluoromethanesulfonate or nonafluorobutanesulfonate; triphenylsulfonium trifluoromethanesulfonate, heptafluoropropanesulfonate or nonafluorobutanesulfonate; tri(4-methylphenyl)sulfonium trifluoromethanesulfonate, heptafluoropropanesulfonate or nonafluorobutanesulfonate; dimethyl(4-hydroxynaphthyl)sulfonium trifluoromethanesulfonate, heptafluoropropanesulfonate or nonafluorobutanesulfonate; monophenyldimethylsulfonium trifluoromethanesulfonate, heptafluoropropanesulfonate or nonafluorobutanesulfonate; diphenylmonomethylsulfonium trifluoromethanesulfonate, heptafluoropropanesulfonate or nonafluorobutanesulfonate; (4-methylphenyl)diphenylsulfonium trifluoromethanesulfonate, heptafluoropropanesulfonate or nonafluorobutanesulfonate; (4-methoxyphenyl)diphenylsulfonium trifluoromethanesulfonate, heptafluoropropanesulfonate or nonafluorobutanesulfonate; tri(4-tert-butyl)phenylsulfonium trifluoromethanesulfonate, heptafluoropropanesulfonate or nonafluorobutanesulfonate; diphenyl(1-(4-methoxy)naphthyl)sulfonium trifluoromethanesulfonate, heptafluoropropanesulfonate or nonafluorobutanesulfonate; di(1-naphthyl)phenylsulfonium trifluoromethanesulfonate, heptafluoropropanesulfonate or nonafluorobutanesulfonate; 1-phenyltetrahydrothiophenium trifluoromethanesulfonate, heptafluoropropanesulfonate or nonafluorobutanesulfonate; 1-(4-methylphenyl)tetrahydrothiophenium trifluoromethanesulfonate, heptafluoropropanesulfonate or nonafluorobutanesulfonate; 1-(3,5-dimethyl-4-hydroxyphenyl)tetrahydrothiophenium trifluoromethanesulfonate, heptafluoropropanesulfonate or nonafluorobutanesulfonate; 1-(4-methoxynaphthalene-1-yl)tetrahydrothiophenium trifluoromethanesulfonate, heptafluoropropanesulfonate or nonafluorobutanesulfonate; 1-(4-ethoxynaphthalene-1-yl)tetrahydrothiophenium trifluoromethanesulfonate, heptafluoropropanesulfonate or nonafluorobutanesulfonate; 1-(4-n-butoxynaphthalene-1-yl)tetrahydrothiophenium trifluoromethanesulfonate, heptafluoropropanesulfonate or nonafluorobutanesulfonate; 1-phenyltetrahydrothiopyranium trifluoromethanesulfonate, heptafluoropropanesulfonate or nonafluorobutanesulfonate; 1-(4-hydroxyphenyl)tetrahydrothiopyranium trifluoromethanesulfonate, heptafluoropropanesulfonate or nonafluorobutanesulfonate; 1-(3,5-dimethyl-4-hydroxyphenyl)tetrahydrothiopyranium trifluoromethanesulfonate, heptafluoropropanesulfonate or nonafluorobutanesulfonate; and 1-(4-methylphenyl)tetrahydrothiopyranium trifluoromethanesulfonate, heptafluoropropanesulfonate or nonafluorobutanesulfonate.

It is also possible to use onium salts in which the anion moiety of these onium salts are replaced by methanesulfonate, n-propanesulfonate, n-butanesulfonate, or n-octanesulfonate.

Further, onium salt-based acid generators in which the anion moiety in general formula (b-1) or (b-2) is replaced by an anion moiety represented by general formula (b-3) or (b-4) (the cation moiety is the same as (b-1) or (b-2)) may also be used.

Furthermore, as an onium salt-based acid generator, a sulfonium salt having a cation moiety represented by general formula (b-5) or (b-6) shown below may be used.

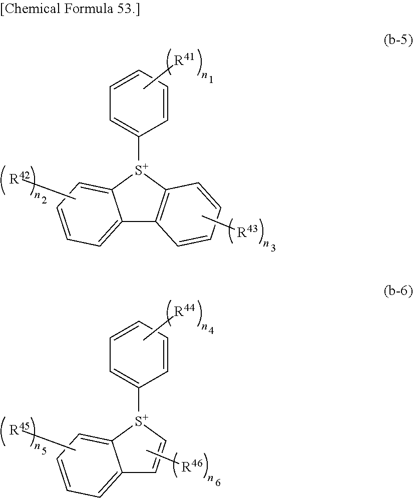

In formulas (b-5) and (b-6) above, each of R⁴¹ to R⁴⁶ independently represents an alkyl group, an acetyl group, an alkoxy group, a carboxy group, a hydroxyl group or a hydroxyalkyl group; each of n₁ to n₅ independently represents an integer of 0 to 3; and n₆ represents an integer of 0 to 2.

With respect to R⁴¹ to R⁴⁶, the alkyl group is preferably an alkyl group of 1 to 5 carbon atoms, more preferably a linear or branched alkyl group, and most preferably a methyl group, ethyl group, propyl group, isopropyl group, n-butyl group or tert-butyl group.

The alkoxy group is preferably an alkoxy group of 1 to 5 carbon atoms, more preferably a linear or branched alkoxy group, and most preferably a methoxy group or ethoxy group.

The hydroxyalkyl group is preferably the aforementioned alkyl group in which one or more hydrogen atoms have been substituted with hydroxy groups, and examples thereof include a hydroxymethyl group, a hydroxyethyl group and a hydroxypropyl group.

If there are two or more of an individual R⁴¹ to R⁴⁶ group, as indicated by the corresponding value of n₁ to n₆, then the two or more of the individual R⁴¹ to R⁴⁶ group may be the same or different from each other.

n₁ is preferably 0 to 2, more preferably 0 or 1, and still more preferably 0.

It is preferable that n₂ and n₃ each independently represent 0 or 1, and more preferably 0.

n₄ is preferably 0 to 2, and more preferably 0 or 1.

n₅ is preferably 0 or 1, and more preferably 0.

n₆ is preferably 0 or 1, and more preferably 1.

The anion moiety of the sulfonium salt having a cation moiety represented by general formula (b-5) or (b-6) is not particularly limited, and the same anion moieties for onium salt-based acid generators which have been proposed may be used. Examples of such anion moieties include fluorinated alkylsulfonic acid ions such as anion moieties (R⁴″SO₃⁻) for onium salt-based acid generators represented by general formula (b-1) or (b-2) shown above; and anion moieties represented by general formula (b-3) or (b-4) shown above. Among these, a fluorinated alkylsulfonate ion is preferable, a fluorinated alkylsulfonate ion of 1 to 4 carbon atoms is more preferable, and a linear perfluoroalkylsulfonate ion of 1 to 4 carbon atoms is particularly desirable. Specific examples thereof include a trifluoromethylsulfonate ion, a heptafluoro-n-propanesulfonate ion and a nonafluoro-n-butanesulfonate ion.

In the present description, an oximesulfonate-based acid generator is a compound having at least one group represented by general formula (B-1) shown below, and has a feature of generating acid by irradiation. Such oximesulfonate acid generators are widely used for a chemically amplified resist composition, and can be appropriately selected.

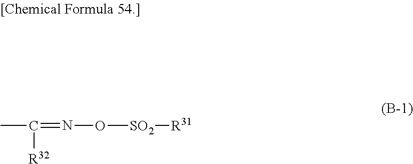

In the formula, each of R³¹ and R³² independently represents an organic group.

The organic group for R³¹ and R³² refers to a group containing a carbon atom, and may include atoms other than carbon atoms (e.g., a hydrogen atom, an oxygen atom, a nitrogen atom, a sulfur atom, a halogen atom (such as a fluorine atom and a chlorine atom) and the like).

As the organic group for R³¹, a linear, branched, or cyclic alkyl group or aryl group is preferable. The alkyl group or the aryl group may have a substituent. The substituent is not particularly limited, and examples thereof include a fluorine atom and a linear, branched, or cyclic alkyl group having 1 to 6 carbon atoms. The alkyl group or the aryl group “has a substituent” means that part or all of the hydrogen atoms of the alkyl group or the aryl group is substituted with a substituent.

The alkyl group preferably has 1 to 20 carbon atoms, more preferably 1 to 10 carbon atoms, still more preferably 1 to 8 carbon atoms, still more preferably 1 to 6 carbon atoms, and most preferably 1 to 4 carbon atoms. As the alkyl group, a partially or completely halogenated alkyl group (hereinafter, sometimes referred to as a “halogenated alkyl group”) is particularly desirable. The “partially halogenated alkyl group” refers to an alkyl group in which part of the hydrogen atoms are substituted with halogen atoms and the “completely halogenated alkyl group” refers to an alkyl group in which all of the hydrogen atoms are substituted with halogen atoms. Examples of halogen atoms include fluorine atoms, chlorine atoms, bromine atoms and iodine atoms, and fluorine atoms are particularly desirable. In other words, the halogenated alkyl group is preferably a fluorinated alkyl group.

The aryl group preferably has 4 to 20 carbon atoms, more preferably 4 to 10 carbon atoms, and most preferably 6 to 10 carbon atoms. As the aryl group, partially or completely halogenated aryl group is particularly desirable. The “partially halogenated aryl group” refers to an aryl group in which some of the hydrogen atoms are substituted with halogen atoms and the “completely halogenated aryl group” refers to an aryl group in which all of hydrogen atoms are substituted with halogen atoms.

As R³¹, an alkyl group of 1 to 4 carbon atoms which has no substituent or a fluorinated alkyl group of 1 to 4 carbon atoms is particularly desirable.

As the organic group for R³², a linear, branched, or cyclic alkyl group, aryl group, or cyano group is preferable. Examples of the alkyl group and the aryl group for R³² include the same alkyl groups and aryl groups as those described above for R³¹.

As R³², a cyano group, an alkyl group of 1 to 8 carbon atoms having no substituent or a fluorinated alkyl group of 1 to 8 carbon atoms is particularly desirable.

Preferred examples of the oxime sulfonate acid generator include compounds represented by general formula (B-2) or (B-3) shown below.

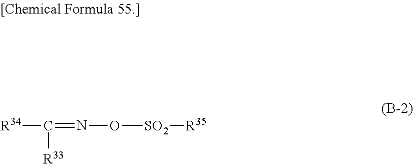

In the formula, R³³ represents a cyano group, an alkyl group having no substituent or a halogenated alkyl group; R³⁴ represents an aryl group; and R³⁵ represents an alkyl group having no substituent or a halogenated alkyl group.

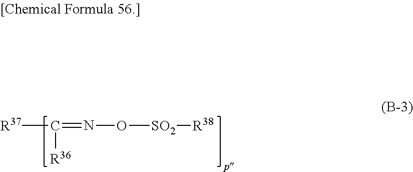

In the formula, R³⁶ represents a cyano group, an alkyl group having no substituent or a halogenated alkyl group; R³⁷ represents a divalent or trivalent aromatic hydrocarbon group; R³⁸ represents an alkyl group having no substituent or a halogenated alkyl group; and p″ represents 2 or 3.

In general formula (B-2), the alkyl group having no substituent or the halogenated alkyl group for R³³ preferably has 1 to 10 carbon atoms, more preferably 1 to 8 carbon atoms, and most preferably 1 to 6 carbon atoms.

As R³³, a halogenated alkyl group is preferable, and a fluorinated alkyl group is more preferable.

The fluorinated alkyl group for R³³ preferably has 50% or more of the hydrogen atoms thereof fluorinated, more preferably 70% or more, and most preferably 90% or more.

Examples of the aryl group for R³⁴ include groups in which one hydrogen atom has been removed from an aromatic hydrocarbon ring, such as a phenyl group, a biphenyl group, a fluorenyl group, a naphthyl group, an anthryl group, and a phenanthryl group, and heteroaryl groups in which some of the carbon atoms constituting the ring(s) of these groups are substituted with hetero atoms such as an oxygen atom, a sulfur atom, and a nitrogen atom. Of these, a fluorenyl group is preferable.

The aryl group for R³⁴ may have a substituent such as an alkyl group of 1 to 10 carbon atoms, a halogenated alkyl group, or an alkoxy group. The alkyl group and halogenated alkyl group as the substituent preferably has 1 to 8 carbon atoms, and more preferably 1 to 4 carbon atoms. Further, the halogenated alkyl group is preferably a fluorinated alkyl group.

The alkyl group having no substituent or the halogenated alkyl group for R³⁵ preferably has 1 to 10 carbon atoms, more preferably 1 to 8 carbon atoms, and most preferably 1 to 6 carbon atoms.

As R³⁵, a halogenated alkyl group is preferable, and a fluorinated alkyl group is more preferable.

In terms of enhancing the strength of the acid generated, the fluorinated alkyl group for R³⁵ preferably has 50% or more of the hydrogen atoms fluorinated, more preferably 70% or more, still more preferably 90% or more. A completely fluorinated alkyl group in which 100% of the hydrogen atoms are substituted with fluorine atoms is particularly desirable.

In general formula (B-3), as the alkyl group having no substituent and the halogenated alkyl group for R³⁶, the same alkyl group having no substituent and the halogenated alkyl group described above for R³³ can be used.

Examples of the divalent or trivalent aromatic hydrocarbon group for R³⁷ include groups in which one or two hydrogen atoms have been removed from the aryl group for R³⁴.

As the alkyl group having no substituent or the halogenated alkyl group for R³⁸, the same one as the alkyl group having no substituent or the halogenated alkyl group for R³⁵ can be used.

p″ is preferably 2.

Specific examples of suitable oxime sulfonate acid generators include α-(p-toluenesulfonyloxyimino)-benzyl cyanide, α-(p-chlorobenzenesulfonyloxyimino)-benzyl cyanide, α-(4-nitrobenzenesulfonyloxyimino)-benzyl cyanide, α-(4-nitro-2-trifluoromethylbenzenesulfonyloxyimino)-benzyl cyanide, α-(benzenesulfonyloxyimino)-4-chlorobenzyl cyanide, α-(benzenesulfonyloxyimino)-2,4-dichlorobenzyl cyanide, α-(benzenesulfonyloxyimino)-2,6-dichlorobenzyl cyanide, α-(benzenesulfonyloxyimino)-4-methoxybenzyl cyanide, α-(2-chlorobenzenesulfonyloxyimino)-4-methoxybenzyl cyanide, α-(benzenesulfonyloxyimino)-thien-2-yl acetonitrile, α-(4-dodecylbenzenesulfonyloxyimino)benzyl cyanide, α-[(p-toluenesulfonyloxyimino)-4-methoxyphenyl]acetonitrile, α-[(dodecylbenzenesulfonyloxyimino)-4-methoxyphenyl]acetonitrile, α-(tosyloxyimino)-4-thienyl cyanide, α-(methylsulfonyloxyimino)-1-cyclopentenyl acetonitrile, α-(methylsulfonyloxyimino)-1-cyclohexenyl acetonitrile, α-(methylsulfonyloxyimino)-1-cycloheptenylacetonitrile, α-(methylsulfonyloxyimino)-1-cyclooctenyl acetonitrile, α-(trifluoromethylsulfonyloxyimino)-1-cyclopentenyl acetonitrile, α-(trifluoromethylsulfonyloxyimino)-cyclohexyl acetonitrile, α-(ethylsulfonyloxyimino)-ethyl acetonitrile, α-(propylsulfonyloxyimino)-propyl acetonitrile, α-(cyclohexylsulfonyloxyimino)-cyclopentyl acetonitrile, α-(cyclohexylsulfonyloxyimino)-cyclohexyl acetonitrile, α-(cyclohexylsulfonyloxyimino)-1-cyclopentenyl acetonitrile, α-(ethylsulfonyloxyimino)-1-cyclopentenyl acetonitrile, α-(isopropylsulfonyloxyimino)-1-cyclopentenyl acetonitrile, α-(n-butylsulfonyloxyimino)-1-cyclopentenyl acetonitrile, α-(ethylsulfonyloxyimino)-1-cyclohexenyl acetonitrile, α-(isopropylsulfonyloxyimino)-1-cyclohexenyl acetonitrile, α-(n-butylsulfonyloxyimino)-1-cyclohexenyl acetonitrile, α-(methylsulfonyloxyimino)-phenyl acetonitrile, α-(methylsulfonyloxyimino)-p-methoxyphenyl acetonitrile, α-(trifluoromethylsulfonyloxyimino)-phenyl acetonitrile, α-(trifluoromethylsulfonyloxyimino)-p-methoxyphenylacetonitrile, α-(ethylsulfonyloxyimino)-p-methoxyphenyl acetonitrile, α-(propylsulfonyloxyimino)-p-methylphenyl acetonitrile, and α-(methylsulfonyloxyimino)-p-bromophenyl acetonitrile.

Further, oxime sulfonate-based acid generators disclosed in Japanese Unexamined Patent Application, First Publication No. Hei 9-208554 (Chemical Formulas 18 and 19 shown in paragraphs [0012] to [0014]) and oxime sulfonate-based acid generators disclosed in WO 20041074242A2 (Examples 1 to 40 described at pages 65 to 85) may be preferably used.

Furthermore, as preferable examples, the following can be used.

Among the aforementioned compounds, the following 4 compounds are preferable.

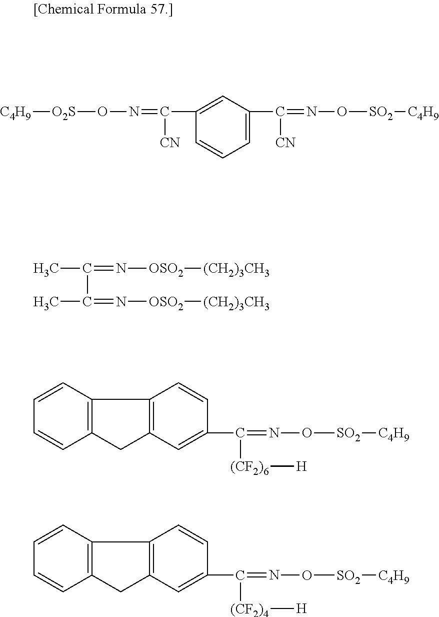

Of the aforementioned diazomethane acid generators, specific examples of suitable bisalkyl or bisaryl sulfonyl diazomethanes include bis(isopropylsulfonyl)diazomethane, bis(p-toluenesulfonyl)diazomethane, bis(1,1-dimethylethylsulfonyl)diazomethane, bis(cyclohexylsulfonyl)diazomethane, and bis(2,4-dimethylphenylsulfonyl)diazomethane.

Further, diazomethane acid generators disclosed in Japanese Unexamined Patent Application, First Publication No. Hei 11-035551, Japanese Unexamined Patent Application, First Publication No. Hei 11-035552 and Japanese Unexamined Patent Application, First Publication No. Hei 11-035573 may be preferably used.

Furthermore, as examples of poly(bis-sulfonyl)diazomethanes, those disclosed in Japanese Unexamined Patent Application, First Publication No. Hei 11-322707, including 1,3-bis(phenylsulfonyldiazomethylsulfonyl)propane, 1,4-bis(phenylsulfonyldiazomethylsulfonyl)butane, 1,6-bis(phenylsulfonyldiazomethylsulfonyl)hexane, 1,10-bis(phenylsulfonyldiazomethylsulfonyl)decane, 1,2-bis(cyclohexylsulfonyldiazomethylsulfonyl)ethane, 1,3-bis(cyclohexylsulfonyldiazomethylsulfonyl)propane, 1,6-bis(cyclohexylsulfonyldiazomethylsulfonyl)hexane, and 1,10-bis(cyclohexylsulfonyldiazomethylsulfonyl)decane, may be given.

As the component (B2), one type of acid generator may be used, or two or more types may be used in combination.

In the resist composition of the present invention, the amount of the component (B) relative to 100 parts by weight of the component (A) is preferably 0.5 to 30 parts by weight, and more preferably 1 to 20 parts by weight. When the amount of the component (B) is within the above-mentioned range, formation of a resist pattern can be satisfactorily performed. Further, by virtue of the above-mentioned range, a uniform solution can be obtained and the storage stability becomes satisfactory.

<Component (D)>

In the resist composition of the present invention, in order to improve the resist pattern shape and the post exposure stability of the latent image formed by the pattern-wise exposure of the resist layer, it is preferable to add a nitrogen-containing organic compound (D) (hereafter referred to as “component (D)”) as an optional component.

A multitude of these components (D) have already been proposed, and any of these known compounds may be used, although an aliphatic amine, and particularly a secondary aliphatic amine or tertiary aliphatic amine is preferable. In the present description and claims, the term “aliphatic” is a relative concept used in relation to the term “aromatic”, and defines a group or compound that has no aromaticity.

The term “aliphatic cyclic group” refers to a monocyclic group or polycyclic group that has no aromaticity. An aliphatic amine is an amine having one or more aliphatic groups, and the aliphatic groups preferably have 1 to 12 carbon atoms.

Examples of these aliphatic amines include amines in which at least one hydrogen atom of ammonia (NH₃) has been substituted with an alkyl group or hydroxyalkyl group of no more than 12 carbon atoms (i.e., alkylamines or alkylalcoholamines), and cyclic amines.

Specific examples of alkylamines and alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-hexylamine, tri-n-pentylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine. Among these, trialkylamines of 5 to 10 carbon atoms are preferable, tri-n-pentylamine or tri-n-octylamine is more preferable, and tri-n-pentylamine is particularly desirable.

Examples of the cyclic amine include heterocyclic compounds containing a nitrogen atom as a hetero atom. The heterocyclic compound may be a monocyclic compound (aliphatic monocyclic amine), or a polycyclic compound (aliphatic polycyclic amine).

Specific examples of the aliphatic monocyclic amine include piperidine, and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine and triethanolamine triacetate, and triethanolamine triacetate is preferable.

Further, as the component (D), an aromatic amine may be used.

Examples of aromatic amines include aniline, pyridine, 4-dimethylaminopyridine, pyrrole, indole, pyrazole, imidazole and derivatives thereof, as well as diphenylamine, triphenylamine, tribenzylamine, 2,6-diisopropylaniline and N-tert-butoxycarbonylpyrrolidine.

These compounds can be used either alone, or in combinations of two or more different compounds.

The component (D) is typically used in an amount within a range from 0.01 to 5.0 parts by weight, relative to 100 parts by weight of the component (A).

<Optional Components>

[Component (E)]

Furthermore, in the resist composition of the present invention, for preventing any deterioration in sensitivity, and improving the resist pattern shape and the post exposure stability of the latent image formed by the pattern-wise exposure of the resist layer, at least one compound (E) (hereafter referred to as the component (E)) selected from the group consisting of an organic carboxylic acid, or a phosphorus oxo acid or derivative thereof can be added.

Examples of suitable organic carboxylic acids include acetic acid, malonic acid, citric acid, malic acid, succinic acid, benzoic acid, and salicylic acid.

Examples of phosphorus oxo acids or derivatives thereof include phosphoric acid, phosphonic acid and phosphinic acid. Among these, phosphonic acid is particularly desirable.

Examples of oxo acid derivatives include esters in which a hydrogen atom within the above-mentioned oxo acids is substituted with a hydrocarbon group. Examples of the hydrocarbon group include an alkyl group of 1 to 5 carbon atoms and an aryl group of 6 to 15 carbon atoms.

Examples of phosphoric acid derivatives include phosphoric acid esters such as di-n-butyl phosphate and diphenyl phosphate.

Examples of phosphonic acid derivatives include phosphonic acid esters such as dimethyl phosphonate, di-n-butyl phosphonate, phenylphosphonic acid, diphenyl phosphonate and dibenzyl phosphonate.

Examples of phosphinic acid derivatives include phosphinic acid esters such as phenylphosphinic acid.

As the component (E), one type may be used alone, or two or more types may be used in combination.

As the component (E), an organic carboxylic acid is preferable, and salicylic acid is particularly desirable.

The component (E) is typically used in an amount within a range from 0.01 to 5.0 parts by weight, relative to 100 parts by weight of the component (A).

If desired, other miscible additives can also be added to the resist composition of the present invention. Examples of such miscible additives include additive resins for improving the performance of the resist film, surfactants for improving the applicability, dissolution inhibitors, plasticizers, stabilizers, colorants, halation prevention agents, and dyes.

[Component (S)]

The resist composition for immersion exposure according to the present invention can be prepared by dissolving the materials for the resist composition in an organic solvent (hereafter, frequently referred to as “component (S)”).

The component (S) may be any organic solvent which can dissolve the respective components to give a uniform solution, and one or more kinds of any organic solvent can be appropriately selected from those which have been conventionally known as solvents for a chemically amplified resist.

Examples thereof include lactones such as γ-butyrolactone; ketones such as acetone, methyl ethyl ketone, cyclohexanone, methyl-n-pentyl ketone, methyl isopentyl ketone, and 2-heptanone; polyhydric alcohols, such as ethylene glycol, diethylene glycol, propylene glycol and dipropylene glycol; compounds having an ester bond, such as ethylene glycol monoacetate, diethylene glycol monoacetate, propylene glycol monoacetate, and dipropylene glycol monoacetate; polyhydric alcohol derivatives including compounds having an ether bond, such as a monoalkylether (e.g., monomethylether, monoethylether, monopropylether or monobutylether) or monophenylether of any of these polyhydric alcohols or compounds having an ester bond (among these, propylene glycol monomethyl ether acetate (PGMEA) and propylene glycol monomethyl ether (PGME) are preferable); cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; and aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene and mesitylene.

These solvents can be used individually, or in combination as a mixed solvent.

Among these, propylene glycol monomethyl ether acetate (PGMEA), propylene glycol monomethyl ether (PGME) and ethyl lactate (EL) and γ-butyrolactone are preferable.

Further, among the mixed solvents, a mixed solvent obtained by mixing PGMEA with a polar solvent is preferable. The mixing ratio (weight ratio) of the mixed solvent can be appropriately determined, taking into consideration the compatibility of the PGMEA with the polar solvent, but is preferably in the range of 1:9 to 9:1, more preferably from 2:8 to 8:2.

Specifically, when EL is mixed as the polar solvent, the PGMEA:EL weight ratio is preferably from 1:9 to 9:1, and more preferably from 2:8 to 8:2. Alternatively, when PGME is mixed as the polar solvent, the PGMEA:PGME weight ratio is preferably from 1:9 to 9:1, more preferably from 2:8 to 8:2, and still more preferably 3:7 to 7:3.

Further, as the component (S), a mixed solvent of at least one of PGMEA and EL with γ-butyrolactone is also preferable. The mixing ratio (former:latter) of such a mixed solvent is preferably from 70:30 to 95:5.

Furthermore, as the component (S), a mixed solvent containing the aforementioned PGMEA/PGME mixed solvent and γ-butyrolactone is also preferable. In this case, the mixing ratio (former:latter) of such a mixed solvent is preferably from 99.9:0.1 to 80:20, more preferably 99.9:0.1 to 90:10, and most preferably 99:9:0.1 to 95:5.

By ensuring the above-mentioned range, the rectangularity of the resist pattern is improved.

The amount of the organic solvent is not particularly limited, and is appropriately adjusted to a concentration which enables coating of a coating solution to a substrate, depending on the thickness of the coating film. In general, the organic solvent is used in an amount such that the solid content of the resist composition becomes within the range from 2 to 20% by weight, and preferably from 5 to 15% by weight.

In the present invention, an acid generator (B1) including a compound represented by the aforementioned general formula (b1) is used. By virtue of having one naphthalene ring in the structure thereof, the component (B1) exhibits improved transparency in the vicinity of 193 nm, i.e., ArF excimer laser. Further, by virtue of having the aforementioned substituent introduced at the first position of the naphthalene ring, the affinity of the component (B1) for the resin component (component (A)) is improved. As a result, it is presumed that the component (B1) is more uniformly distributed within the resist than conventional acid generators, thereby achieving excellent lithography properties.

For the reasons as described above, when the resist composition of the present invention is a positive resist composition, it is presumed that a line and space pattern (L/S) pattern having excellent properties with respect to line width roughness (LWR), exposure (EL) margin, mask error factor (MEF) and resist pattern shape can be formed.

The resist composition of the present invention can be preferably used in a method of forming a resist pattern including an immersion exposure step, and excellent lithography properties can be obtained. Further, the resist composition of the present invention can be preferably used for forming an upper-layer resist film in a method of forming a resist pattern including a step of forming a 3-layered resist laminate, and excellent lithography properties can be obtained.

<<Method of Forming a Resist Pattern>>

Next, a method of forming a resist pattern according to a second aspect of the present invention will be described.

The method of forming a resist pattern according to the present invention includes forming a resist film using a resist composition according to the first aspect, subjecting the resist film to exposure, and subjecting the resist film to alkali developing to form a resist pattern.

The method for forming a resist pattern according to the present invention can be performed, for example, as follows.

Firstly, a resist composition according to the first aspect of the present invention is applied to a substrate using a spinner or the like, and a prebake (post applied bake (PAB)) is conducted under temperature conditions of 80 to 150° C. for 40 to 120 seconds, preferably 60 to 90 seconds to form a resist film. Then, for example, using an ArF exposure apparatus or the like, the resist film is selectively exposed to an ArF excimer laser beam through a desired mask pattern, followed by post exposure bake (PEB) under temperature conditions of 80 to 150° C. for 40 to 120 seconds, preferably 60 to 90 seconds. Subsequently, developing is conducted using an alkali developing solution such as a 0.1 to 10% by weight aqueous solution of tetramethylammonium hydroxide, preferably followed by rinsing with pure water, and drying. If desired, bake treatment (post bake) can be conducted following the developing. In this manner, a resist pattern that is faithful to the mask pattern can be obtained.

The substrate is not specifically limited and a conventionally known substrate can be used. For example, substrates for electronic components, and such substrates having wiring patterns formed thereon can be used. Specific examples of the material of the substrate include metals such as silicon wafer, copper, chromium, iron and aluminum; and glass. Suitable materials for the wiring pattern include copper, aluminum, nickel, and gold.

Further, as the substrate, any one of the above-mentioned substrates provided with an inorganic and/or organic film on the surface thereof may be used. As the inorganic film, an inorganic antireflection film (inorganic BARC) can be used. As the organic film, an organic antireflection film (organic BARC) can be used.

The wavelength to be used for exposure is not particularly limited and the exposure can be conducted using radiation such as ArF excimer laser, KrF excimer laser, F₂ excimer laser, extreme ultraviolet rays (EUV), vacuum ultraviolet rays (VUV), electron beam (EB), X-rays, and soft X-rays. The resist composition of the present invention is effective to KrF excimer laser, ArF excimer laser, EB and EUV, and particularly effective to ArF excimer laser. Further, the resist composition of the present invention is also effective to immersion exposure.

### EXAMPLES

As follows is a description of examples of the present invention, although the scope of the present invention is in no way limited by these examples. In the NMR analysis, the internal standard for ¹H-NMR is tetramethylsilane (TMS), and the internal standard for ¹⁹F-NMR is hexafluorobenzene (the peak of hexafluorobenzene was regarded as −160 ppm).

### Synthesis Example 1

### Synthesis of Compound (A)

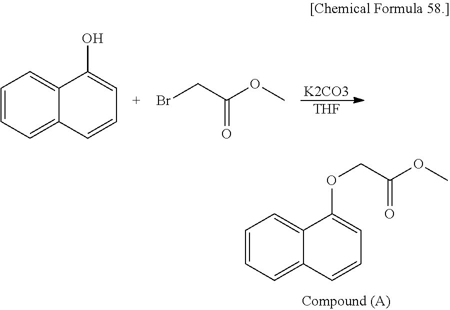

In a nitrogen atmosphere, 25 g of THF was added to 5 g of 1-naphthol, and 23.96 g of potassium carbonate was further added. Then, 6.36 g of methyl bromoacetate was dropwise added to the resulting mixture, and reflux was conducted for 5 hours while stirring. After the stirring, the mixture was separated by filtration, and the filtrate was washed with 37.5 g of water 4 times. Thereafter, the resultant was concentrated using a rotary evaporator, followed by distillation under reduced pressure (0.29 kPa, 152° C.), thereby obtaining 4 g of a compound (A).

The compound (A) was analyzed by NMR.

¹H-NMR (DMSO, 400 MHz): δ (ppm)=8.23-8.27 (m, 1H, Naph), 7.87-7.91 (m, 1H, Naph), 7.51-7.57 (m 3H, Naph), 7.38-7.51 (m, 1H, Naph), 6.91-6.92 (m, 1H Naph), 1.99 (s, 2H, CH2), 3.72 (s, 3HCH3).

From the results, it was confirmed that the compound (A) had a structure as shown above.

### Example 1

### Synthesis of Compound PAG-1

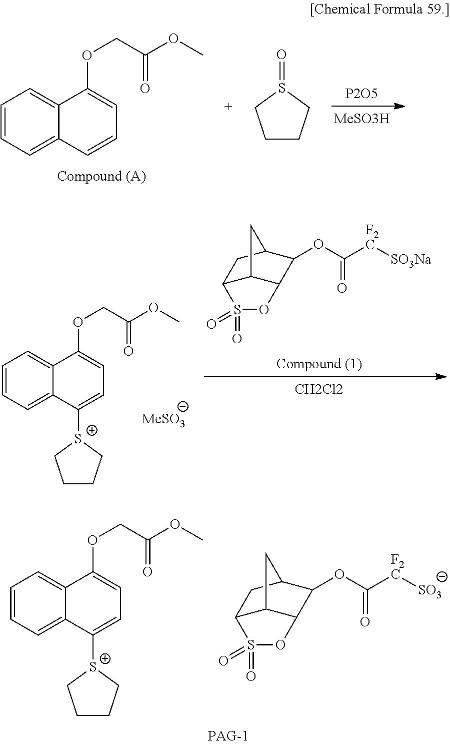

To a solution containing 17.63 g of methanesulfonic acid and 1.25 g of diphosphorous pentaoxide was added 1.92 g of the compound (A) and 0.923 g of tetramethylene sulfoxide at 25° C. After stirring at 25° C. for 3 hours, the reaction liquid was dropwise added to a solution containing 26.44 g of t-butylether and 34.37 g of water. After washing by liquid separation, the aqueous phase was washed with 26.44 g of t-butylether twice. To the obtained aqueous phase was added 55 g of dichloromethane and 3.1 g of a compound (1), followed by stirring for 1 hour. After the stirring, the organic solvent phase was washed with 27.5 g of a 1% HCl solution, followed by washing with 27.5 g of water 3 times. The resultant was concentrated and solidified, thereby obtaining 2.82 g of a compound PAG-1.

The compound PAG-1 was analyzed by NMR.

¹H-NMR (DMSO, 400 MHz): δ (ppm)=8.38-8.45 (m, 2H, Naph), 8.12-8.15 (m, 2H, Naph), 7.76-7.93 (m 2H, Naph), 7.18-7.03 (m, 1H, Naph), 5.21 (s, 2H, CH2), 4.66-4.81 (m, 2H CH, CH), 3.49-4.08 (m, 9H, CH2+CH2+CH3, CH), 1.73-2.48 (m, 9H, CH2+CH2+CH2+CH+CH+CH).

¹⁹F-NMR (DMSO, 376 MHz): δ (ppm)=−107.6

From the results, it was confirmed that the compound PAG-1 had a structure as shown above.

### Example 2 to 22

### Synthesis of Compounds PAG-2 to PAG-22

The same procedure as in Example 1 was performed, except that the compound (M⁺X⁻) was changed to a compound shown in Tables 1 to 8 (equimolar amount). In this manner, products having an anion and a cation as shown in Tables 1 to 8 (compounds PAG-2 to PAG-22) were obtained. Each of the obtained compounds was analyzed by NMR. The results are shown in Tables 1 to 8. In Tables 1 to 8, “↑” indicates that the cation of the compound PAG-2 to PAG-22 is the same as that of the compound PAG-1.

TABLE 1

| Ex- | | | | | |
|---|---|---|---|---|---|
| am- | Com- | | Salt | | |
| ple | pound | NMR | M⁺X⁻ | Cation | Anion |
| 1 | PAG- 1 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 4.78 (m, 1H, CH), 4.66 (t, 1H, CH), 3.75- 4.19 (m, 8H, SCH2 + CH3 + CH),, 3.34 (m, 1H, CH), 1.73-2.60 (m, 9H, CH + SCH2CH2 + CH2),, ¹⁹F-NMR (DMSO-d6, 376 MHz): δ (ppm) = −107.7. | 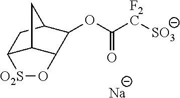 | 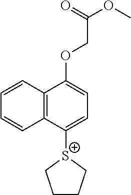 | 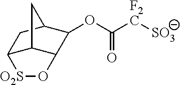 |
| 2 | PAG- 2 | ¹H-NMR (DMSO-d6, 400 MHz): δ (ppm) = 8.42 (m, 2H, ArH), 8.17 (d, 1H, ArH), 7.78- 7.91 (m, 2H, ArH), 7.23 (d, 1H, ArH), 5.26 (s, 2H, CH2), 4.40 (t, 2H, CH2), 3.75-4.21 (m, 9H, CH2 + SCH2 + CH3), 2.29-2.60 (m, 4H, SCH2CH2), 1.61- 1.98 (m, 15H, Adamantane),, ¹⁹F-NMR (DMSO-d6, 376 MHz): δ (ppm) = −106.6. | 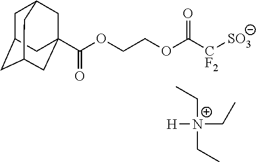 | ↑ | 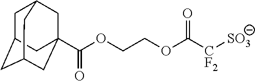 |
| 3 | PAG- 3 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 4.40-4.50 (m, 4H, CH2), 3.75-4.19 (m, 7H, SCH2 + CH3), 2.29-2.60 (m, 4H, SCH2CH2), ¹⁹F-NMR (DMSO-d6, 376 MHz): (ppm) = −106.7, −154.0, −160.0, −161.5. | 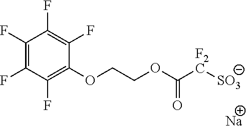 | ↑ | 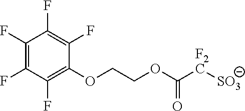 |

TABLE 2

| | Com- | | Salt | |
|---|---|---|---|---|
| Example | pound | NMR | M⁺X⁻ | Cation |
| 4 | PAG- 4 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 4.41 (t, 2H, CH2), 4.23 (t, 2H, CH2), 3.75-4.19 (m, 7H, SCH2 + CH3), 0.79-2.60 (m, 25H, SCH2CH2 + Undcanoyl), ¹⁹F-NMR (DMSO-d6, 376 MHz): δ (ppm) = −106.8. | 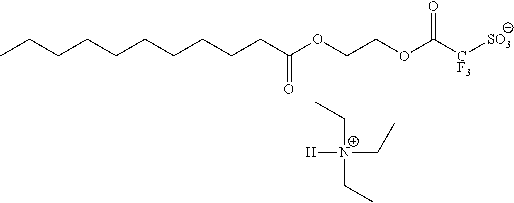 | ↑ |
| 5 | PAG- 5 | ¹H-NMR (DMSO-d6, 400 MHz): δ (ppm) = 8.74-8.82 (m, 2H, Py—H), 8.42 (m, 2H, ArH), 8.17 (d, 1H, ArH), 7.84 (dd, 2H, Py—H), 7.78-7.91 (m, 2H, ArH), 7.23 (d, 1H, ArH), 5.26 (s, 2H, CH2), 4.54- 4.61 (m, 4H, CH2CH2), 3.75-4.19 (m, 7H, SCH2 + CH3), 2.29-2.60 (m, 4H, SCH2CH2), ¹⁹F-NMR (DMSO-d6, 376 MHz): δ (ppm) = −106.5. | 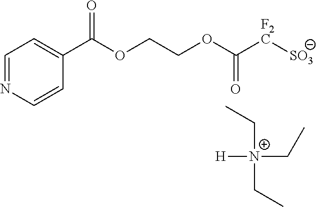 | ↑ |

| Example | Anion |
|---|---|
| 4 | 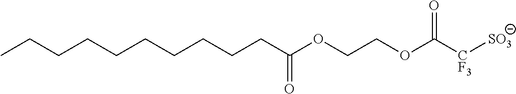 |
| 5 | 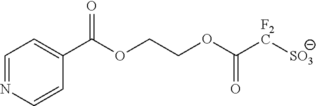 |

TABLE 3

| Ex- | | | | | |
|---|---|---|---|---|---|
| am- | Com- | | Salt | | |
| ple | pound | NMR | M⁺X⁻ | Cation | Anion |
| 6 | PAG- 6 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.46 (t, 1H, oxo-norbornane), 5.26 (s, 2H, CH2), 4.97 (s, 1H, oxo-norbornane), 4.71 (d, 1H, oxo-norbornane), 4.57 (d, 1H, oxo- norbornane), 3.75-4.19 (m, 7H, SCH2 + CH3), 2.29-2.73 (m, 5H, oxo-norbornane + SCH2CH2),, 2.06-2.16 (m, 2H, oxo-norbornane), ¹⁹F-NMR (DMSO-d6, 376 MHz): (ppm) = −107.1. | 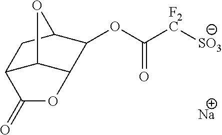 | ↑ | 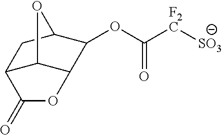 |
| 7 | PAG- 7 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 4.55 (t, 2H, CF2CH2), 3.75-4.19 (m, 7H, SCH2 + CH3), 2.29-2.60 (m, 4H, SCH2CH2), 1.94 (m, 3H, Adamantane), 1.82 (m, 6H, Adamantane), 1.64 (m, 6H, Ad) ¹⁹F-NMR (DMSO-d6, 376 MHz): δ (ppm) = −111.2. | 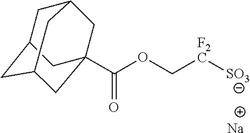 | ↑ | 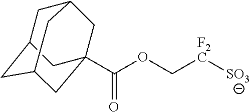 |
| 8 | PAG- 8 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 4.40 (t, 2H, CH2), 3.75-4.20 (m, 9H, SCH2 + CH3 + CH2), 2.29-2.60 (m, 4H, SCH2CH2), 2.05 (s, 2H, CH2), 1.53-1.95 (m, 15H, Adamantane), ¹⁹F-NMR (DMSO-d6, 376 MHz): δ (ppm) = −111.2. | 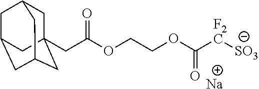 | ↑ | 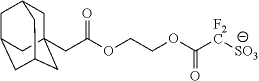 |

TABLE 4

| | | | Salt | | |
|---|---|---|---|---|---|
| Example | Compound | NMR | M⁺X⁻ | Cation | Anion |
| 9 | PAG-9 | ¹H-NMR (DMSO-d6, 400 MHz): δ (ppm) =8.42 (m, 2H, ArH), 8.17 (d, 1H, ArH), 7.78- 7.91 (m, 2H, ArH), 7.23 (d, 1H, ArH), 5.26 (s, 2H, CH2),, 3.75- 4.19 (m, 9H, SCH2 + CH3 + CH2), 2.29-2.60 (m, 4H, SCH2CH2), 1.55-1.87 (m, 15H, Adamantane), ¹⁹F-NMR (DMSO-d6, 378 MHz): δ (ppm) = −77.7. | 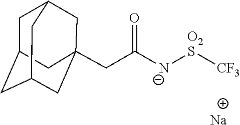 | ↑ | 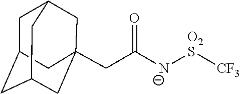 |
| 10 | PAG-10 | ¹H-NMR (DMSO-d6, 400 MHz): δ (ppm) = 8.42 (m, 2H, ArH), 8.17 (d, 1H, ArH), 7.78- 7.91 (m, 2H, ArH), 7.23 (d, 1H, ArH), 5.26 (s, 2H, CH2),, 3.75- 4.19 (m, 7H, SCH2 + CH3), 2.77-2.81 (m, 1H, Cyclohexyl), 2.29-2.60 (m, 4H, SCH2CH2), 2.04-2.08 (m, 2H, Cyclohexyl), 1.73-1.75 (m, 2H, Cyclohexyl), 1.56-1.59 (m, 1H, Cyclohexyl), 1.07-1.33 (m, 5H, Cyclohexyl) ¹⁹F-NMR (DMSO-d6, 376 MHz): δ (ppm) = −74.7 | 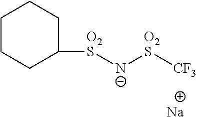 | ↑ | 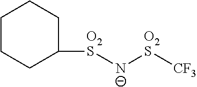 |
| 11 | PAG-11 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 3.75-4.19 (m,7H, SCH2 + CH3), 2.29-2.60 (m, 4H, SCH2CH2), 1.55- 1.88 (m, 15H, Adamantane). ¹⁹F-NMR (DMSO-d6, 376 MHz): δ (ppm) = −74.5. | 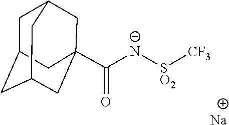 | ↑ | 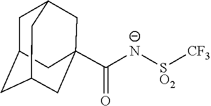 |

TABLE 5

| Ex- | Com- | | Salt | Cat- | |
|---|---|---|---|---|---|
| ample | pound | NMR | M⁺X⁻ | ion | Anion |
| 12 | PAG-12 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 3.75-4.19 (m, 7H, SCH2 + CH3), 2.29-2.60 (m, 4H, SCH2CH2), 2.13 (m, 3H, Adamantane), 1.99 (m, 6H, Adamantane), 1.59 (s, 6H, Adamantane), ¹⁹F-NMR (DMSO-d6, 376 MHz): δ (ppm) = −69.2, −76.0, −112.9. | 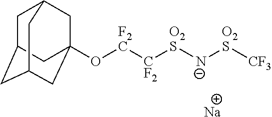 | ↑ | 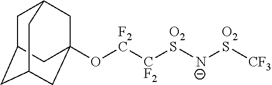 |
| 13 | PAG-13 | ¹H-NMR (DMSO-d6, 400 MHz): δ (ppm) = 8.42 (m, 2H, ArH), 8.17 (d, 1H, ArH), 7.51-7.96 (m, 9H, ArH), 7.23 (d, 1H, ArH), 5.20-5.26 (m, 4H, CH2 + CH2), 3.75-4.19 (m 7H, SCH2 + CH3), 2.29- 2.60 (m, 4H, SCH2CH2), ¹⁹F-NMR (DMSO-d6, 376 MHz): δ (ppm) = −80.5, −113.7. | 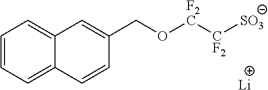 | ↑ | 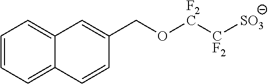 |
| 14 | PAG-14 | ¹H-NMR (DMS0-d6, 400 MHz): δ (ppm) = 8.42 (m, 2H ArH), 8.17 (d, 1H, ArH), 7.78-7.91 (m, 2H, ArH), 7.23 (d, 1H, ArH), 5.26 (s, 2H, CH2), 3.75-4.19 (m, 7H, SCH2 + CH3), 2.29-2.60 (m, 4H, SCH2CH2), 2.09 (s, 3H, Adamantane), 1.96 (s, 6H, Adamantane), 1.56 (s, 6H, Adamantane), ¹⁹F-NMR (DMSO-d6, 376 MHz): δ (ppm) = −70.1, −113.4. | 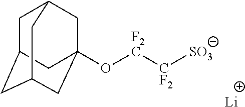 | ↑ | 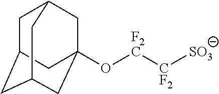 |

TABLE 6

| Example | Compound | NMR | Salt M⁺X⁻ | Cation | Anion |
|---|---|---|---|---|---|
| 15 | PAG-15 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 3.75-4.19 (m, 7H, SCH2 + CH3), 2.88 (d, 1H, CH), 2.17-2.74 (m, 7H, SCH2CH2 + CH), 1.74-1.90 (m, 3H, CH2 + CH), 1.22-1.29 (m, 2H, CH2), 1.03 (s, 3H, CH3), 0.71 (s, 3H, CH3) | 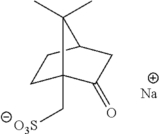 | ↑ | 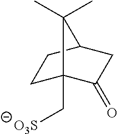 |
| 16 | PAG-16 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 3.75-4.19 (m, 7H, SCH2 + CH3), 2.29-2.60 (m, 4H, SCH2CH2), ¹⁹F-NMR (DMSO-d6, 376 MHz): δ (ppm) = −77.3, −111.5, −118.1, −122.4. | 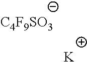 | ↑ | 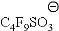 |
| 17 | PAG-17 | ¹H-NMR (DMSO-d6, 400 MHz): δ (ppm) = 8.42 (m, 2H, ArH), 8.17 (d, 1H, ArH), 7.78-7.91 (m, 2H, ArH), 7.23 (d, 1H, ArH), 5.26 (s, 2H, CH2), 3.75-4.19 (m, 7H, SCH2 + CH3), 2.29-2.60 (m, 4H, SCH2CH2), ¹⁹F-NMR (DMSO-d6, 376 MHz): δ (ppm) = −75.0 | 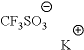 | ↑ | 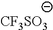 |

TABLE 7

| Ex- | Com- | | | Cat- | |
|---|---|---|---|---|---|
| ample | pound | NMR | Salt M⁺X⁻ | ion | Anion |
| 18 | PAG- 18 | ¹H-NMR (DMSO-d6, 400 MHz): δ (ppm) = 8.42 (m, 2H ArH), 8.17 (d, 1H, ArH), 7.78-7.91 (m, 2H, ArH), 7.23 (d, 1H, ArH), 5.26 (s, 2H, CH2), 3.75-4.19 (m, 7H, SCH2 + CH3), 2.29-2.60 (m, 4H, SCH2CH2), ¹⁹F-NMR (DMSO-d6, 376 MHz): δ (ppm) = −77.3, −112.5, −121.7. | 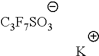 | ↑ | 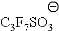 |
| 19 | PAG- 19 | ¹H-NMR (DMSO- d6, 400 MHz): δ (ppm) = 8.42 (m, 2H ArH), 8.17 (d, 1H, ArH), 7.78-7.91 (m, 2H, ArH), 7.23 (d, 1H, ArH), 5.83-5.92 (m, 1H, anion CH), 5.41 (dd, 1H, anion CH), 5.26 (s, 2H, CH2),5.21 (dd, 1H, anion CH), 4.45 (s, 2H, anion CH2), 3.75-4.19 (m, 7H, SCH2 + CH3), 2.29-2.60 (m, 4H, SCH2CH2), ¹⁹F-NMR (DMSO-d6, 376 MHz): δ (ppm) = −80.0, −113.0 | 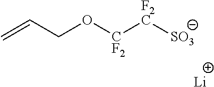 | ↑ | 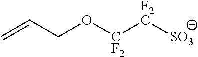 |
| 20 | PAG- 20 | ¹H-NMR (DMSO- d6, 400 MHz): δ (ppm) = 8.42 (m, 2H ArH), 8.17 (d, 1H, ArH), 7.78-7.91 (m, 2H, ArH), 7.23 (d, 1H, ArH), 5.26 (s, 2H, CH2), 4.49-4.51 (m, 2H, O—CH2), 4.30-4.32 (m, 2H, O—CH2), 3.75-4.19 (m, 7H, SCH2 + CH3), 2.29-2.60 (m, 6H, CO—CH2 + SCH2CH2), 1.51-1.56 (m, 2H, CH2), 1.15-1.35 (m, 6H, CH2), 0.87 (t, 3H, CH3) ¹⁹F-NMR (DMSO-d6, 376 MHz): δ (ppm) = −106.7. | 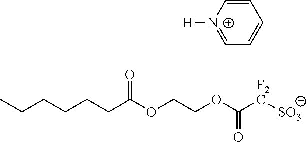 | ↑ | 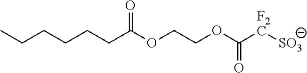 |

TABLE 8

| Ex- | Com- | | Salt | | |
|---|---|---|---|---|---|
| ample | pound | NMR | M⁺X⁻ | Cation | Anion |
| 21 | PAG-21 | ¹H-NMR (DMSO-d6, 400 MHz): δ (ppm) = 8.42 (m, 2H ArH), 8.17 (d, 1H, ArH), 7.78- 7.91 (m, 2H, ArH), 7.23 (d, 1H, ArH), 5.26 (s, 2H, CH2), 4.50-4.54 (m, 4H, OCH2CH2O), 3.75-4.19 (m, 7H, SCH2 + CH3), 3.57 (d, 1H, CH2SO2), 3.36 (sd, 1H, CH2SO2), 2.24-2.60 (m, 6H, SCH2CH2 + camphor), 2.07 (t, 1H, camphor), 1.92-1.99 (m, 2H, camphor), 1.56-1.62 (m, 1H, camphor), 1.42-1.45 (m, 1H, camphor), 1.04 (s, 3H, CH3), 0.84 (s, 3H, CH3), ¹⁹F-NMR (DMSO-d6, 376 MHz): δ (ppm) = −106.5 | 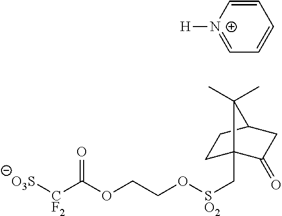 | ↑ | 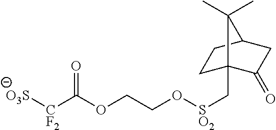 |
| 22 | PAG-22 | ¹H-NMR (DMSO-d6, 400 MHz): δ (ppm) = 8.42 (m, 2H ArH), 8.17 (d, 1H, ArH), 7.78-7.91 (m, 2H, ArH) 7.23 (d, 1H, ArH), 5.26 (s, 2H, CH2), 4.22 (s, 2H, CH2O), 3.75-4.19 (m, 9H, CH2CF2 + SCH2 + CH3), 3.13 (q, 6H, CH2CH3), 2.24-2.60 (m, 6H, SCH2CH2 + Adamantane), 1.53-1.99 (m, 15H, Adamantane + CH3), 1.20 (t, 9H, CH2CH3) ¹⁹F-NMR (DMSO-d6, 376 MHz): δ (ppm) = −111.0. | 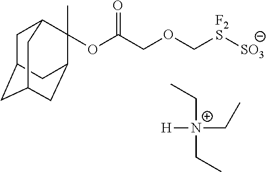 | ↑ | 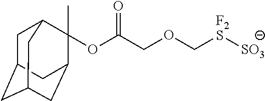 |

### Synthesis Example 2

### Synthesis of Compound (B)

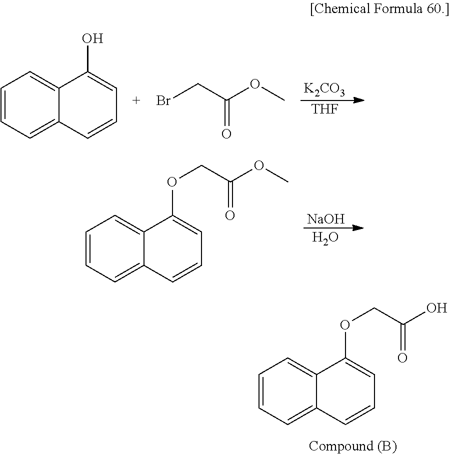

In a nitrogen atmosphere, 250 g of THF was added to 50 g of 1-naphthol, and 23.96 g of potassium carbonate was further added. Then, 63.6 g of methyl bromoacetate was dropwise added to the resulting mixture, and reflux was conducted for 5 hours while stirring. After the stirring, the mixture was separated by filtration, and the filtrate was washed with 250 g of water 4 times. Thereafter, the resultant was concentrated using a rotary evaporator, and 308.5 g of water and 16.80 g of NaOH were added to the concentrated solution, followed by stirring. After confirming the decomposition of the ester, 16.86 g of a 35% aqueous HCl solution and 569.39 g of water were dropwise added to the deposited solution, and stirring was conducted for 30 minutes, followed by filtration, thereby obtaining 48.61 g of a compound (B).

The compound (B) was analyzed by NMR.

¹H-NMR (DMSO, 400 MHz): δ (ppm)=13.15 (br-s, 1H, OH), 8.23 (d, 1H, ArH), 8.89 (d, 1H, ArH), 7.35-7.58 (m, 4H, ArH), 6.88 (d, 1H ArH), 4.91 (s, 2H, CH2).

From the results, it was confirmed that the compound (B) had a structure as shown above.

### Synthesis Example 3

### Synthesis of Compound (C)

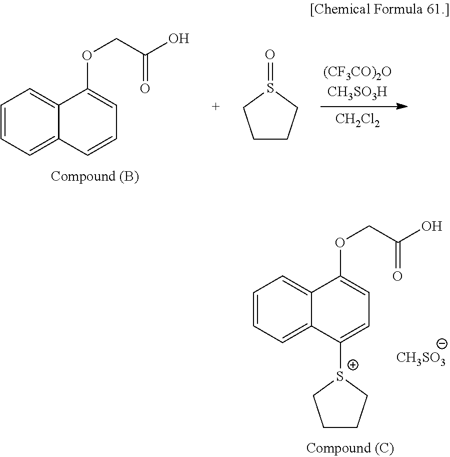

In a nitrogen atmosphere, 1.57 g of methanesulfonic acid was added to 33.2 g of dichloromethane, and cooled down to −30° C. Then, 3.44 g of trifluoroacetic anhydride was added thereto, and 3.32 g of a compound (B) and 4.3 g of tetramethylene sulfoxide were further added. After stirring at −30° C. for 30 minutes, the resultant was further stirred at 3° C. for 5 hours. The reaction liquid was dropwise added to 700 g of hexane and stirred for 30 minutes, followed by filtration. The obtained residue was dried in vacuum, thereby obtaining 4.3 g of a compound (C).

The compound (C) was analyzed by NMR.

¹H-NMR (DMSO, 400 MHz): δ (ppm)=8.32 (m, 2H, ArH), 8.14 (d, 1H, ArH), 7.75-8.93 (m 2H, ArH), 7.18 (d, 1H, ArH), 5.08 (s, 2H CH2), 3.73-4.56 (m, 4H, SCH2), 2.26-2.52 (m, 7H, SCH2CH2, CH3SO3).

From the results, it was confirmed that the compound (C) had a structure as shown above.

### Synthesis Example 4

### Synthesis of Compound (D)

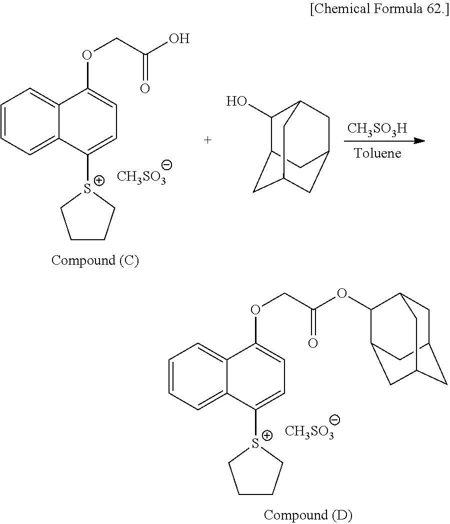

In a nitrogen atmosphere, 8.00 g of the compound (C), 4.15 g of 2-adamantanol, 80.00 g of toluene and 0.0249 g of methanesulfonic acid were added and mixed together, and reflux was conducted for 15 hours while stirring. After cooling the reaction liquid to room temperature, 40 g of water was added, followed by washing with 40 g of t-butylether 3 times. The obtained aqueous solution (aqueous solution of a compound (D)) was used in the next reaction without any analysis.

### Example 23

### Synthesis of Compound PAG-23

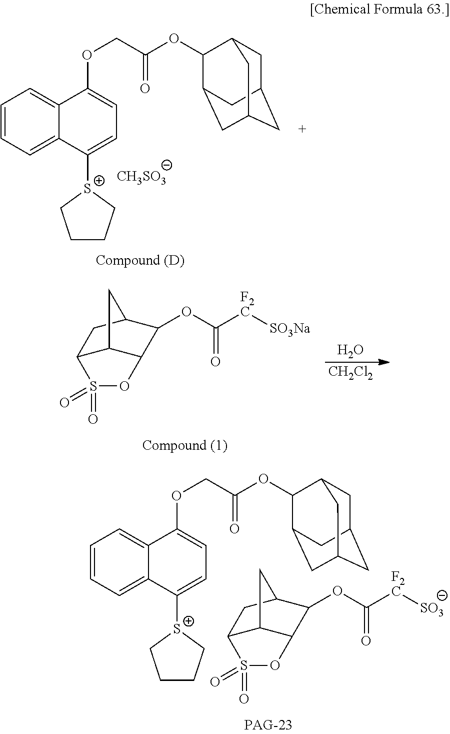

To 6.03 g of the aqueous solution of the compound (D) was added 60.3 g of dichloromethane and 3.91 g of the compound (1), followed by stirring for 1 hour. After the stirring, the resultant was washed with 20.10 g of a 1% aqueous HCl solution, followed by washing with 20.10 g of water 4 times. The obtained organic solution was dropwise added to 600 g of hexane, and stirred for 1 hour. The resultant was subjected to filtration, and the residue was dried, thereby obtaining 7.15 g of a compound PAG-23.

The compound PAG-23 was analyzed by NMR.

¹H-NMR (DMSO, 400 MHz): δ (ppm)=8.41 (m, 2H, ArH), 8.12 (d, 1H, ArH), 7.73-7.93 (m 2H, ArH), 7.19 (d, 1H, ArH), 5.23 (s, 2H CH2), 4.95 (m, 1H, adamantane), 4.78 (m, 1H, CH), 4.66 (t, 1H, CH), 4.03 (m, 2H, CH2S), 3.88 (t, 1H, CH), 3.75 (m, 2H, CH2S), 3.34 (m, 1H, CH), 2.27-2.49 (m, 5H, SCH2CH2+CH), 1.42-2.21 (m, 18H, adamantane+CH2).

From the results, it was confirmed that the compound PAG-23 had a structure as shown above.

### Example 24 to 44

### Synthesis of Compounds PAG-24 to PAG-44

The same procedure as in Example 23 was performed, except that the compound (M⁺X⁻) was changed to a compound shown in Tables 9 to 16 (equimolar amount). In this manner, products having an anion and a cation as shown in Tables 9 to 16 (compounds PAG-24 to PAG-44) were obtained. Each of the obtained compounds was analyzed by NMR. The results are shown in Tables 9 to 16. In Tables 9 to 16, “↑” indicates that the cation of the compound PAG-24 to PAG-44 is the same as that of the compound PAG-23.

TABLE 9

| Ex- | Com- | | Salt | | |
|---|---|---|---|---|---|
| ample | pound | NMR | M⁺X⁻ | Cation | Anion |
| 23 | PAG- 23 | ¹H-NMR (DMSO- d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.78 (m, 1H, CH), 4.66 (t, 1H, CH), 4.03 (m, 2H, CH2S), 3.88 (t, 1H, CH),, 3.75 (m, 2H, CH2S), 3.34 (m, 1H, CH), 2.27-2.49 (m, 5H, CH + SCH2CH2), 1.42-2.21 (m, 18H, CH2 + Adamantane), ¹⁹F-NMR (DMSO-d6, 376 MHz): δ (ppm) = −107.7. | 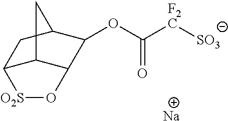 | 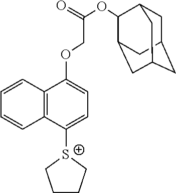 | 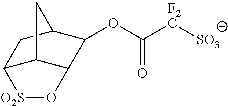 |
| 24 | PAG- 24 | ¹H-HMR (DMSO- d6, 400 MHz): δ (ppm) = 8.41 (m, 2H, ArH), 8.12 (d, 1H ArH), 7.73-7.93 (m, 2H, ArH), 7.19 (d, 1H, ArH), 5.23 (s, 2H, CH2), 4.95 (m, 1H, Adamantane), 4.40 (t, 2H, CH2), 4.21 (t, 2H, CH2), 4.03 (m, 2H, CH2S), 3.75 (m, 2H, CH2S), 2.27-2.43 (m, 4H, SCH2CH2) 1.42- 1.99 (m, 29H, Adamantane), , ¹⁹F-NMR (DMSO-d6, 376 MHz): δ (ppm) = −106.6. | 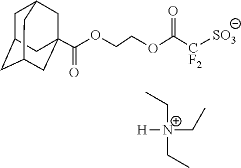 | ↑ | 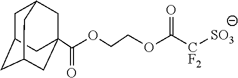 |
| 25 | PAG- 25 | ¹H-NMR (DMSO- d6, 400 MHz): δ (ppm) = 8.41 (m, 2H, ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.40-4.50 (m, 4H, CH2), 4.03 (m, 2H, CH2S), 3.75 (m, 2H, CH2S), 2.27- 2.43 (m, 4H, SCH2CH2), 1.42-1.99 (m, 14H, Adamantane) ¹⁹F-NMR (DMSO- d6, 376 MHz): δ (ppm) = −106.7, −154.0, −160.0, −161.5. | 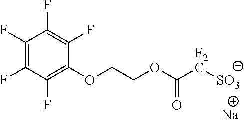 | ↑ | 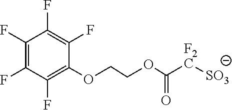 |

TABLE 10

| Ex- | Com- | | Salt | Cat- | |
|---|---|---|---|---|---|
| ample | pound | NMR | M⁺X⁻ | ion | Anion |
| 26 | PAG- 26 | ¹H-NMR (DMSO- d6, 400 MHz): δ (ppm) = 8.41 (m, 2H, ArH), 8.12 (d, 1H, ArH), 7.73-7.93 (m, 2H, ArH) 7.19 (d, 1H, ArH), 5.23 (s, 2H, CH2), 4.95 (m, 1H, Adamantane), 4.41 (t, 2H, CH2), 4.23 (t, 2H, CH2), 4.03 (m, 2H, CH2S), 3.75 (m, 2H, CH2S), 0.79-2.43 (m, 39H, SCH2CH2 + Adamantane + Undecanoyl), ¹⁹F-NMR (DMSO-d6, 376 MHz): δ (ppm) = −106.8. | 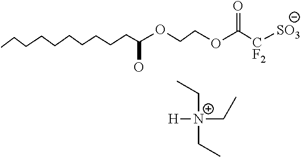 | ↑ | 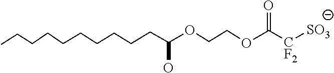 |
| 27 | PAG- 27 | ¹H-NMR (DMSO- d6, 400 MHz): δ (ppm) = 8.74-8.82 (m, 2H, Py—H), 8.41 (m, 2H ArH), 8.12 (d, 1H, ArH), 7.73-7.93 (m, 4H, Py—H + Ar—H),, 7.19 (d, 1H, ArH), 5.23 (s, 2H, CH2), 4.95 (m, 1H, Adamantane), 4.54- 4.61 (m, 4H, CH2CH2), 4.03 (m, 2H, CH2S), 3.75 (m, 2H, CH2S), 2.27-2.43 (m, 4H, SCH2CH2), 1.42- 1.99 (m, 14H, Adamantane), ¹⁹F-NMR (DMSO- d6, 376 MHz): δ (ppm) = −106.5. | 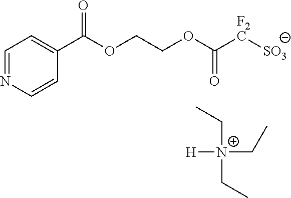 | ↑ | 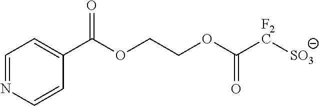 |

TABLE 11

| Ex- | Com- | | Salt | | |
|---|---|---|---|---|---|
| ample | pound | NMR | M⁺X⁻ | Cation | Anion |
| 28 | PAG-28 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.46 (t, 1H, oxo-norbornane), 5.23 (s, 2H, CH2), 4.95-4.97 (m, 2H, oxo-norbornane + Adamantane), 4.71 (d, 1H, oxo-norbornane), 4.57 (d, 1H, oxo-norbornane), 4.03 (m, 2H, CH2S), 3.75 (m, 2H, CH2S), 2.69-2.73 (m, 1H, oxo-norbornane), 2.27-2.43 (m, 4H, SCH2CH2), 2.06-2.16 (m, 2H, oxo-norbornane), 1.42-1.99 (m, 14H, Adamantane) ¹⁹F-NMR (DMSO-d6, 376 MHz): δ (ppm) = −107.1. | 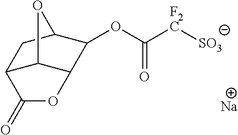 | ↑ | 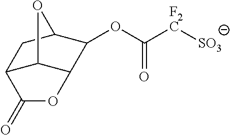 |
| 29 | PAG-29 | ¹H-NMR (DMSO-d6, 400 MHz): δ (ppm) = 8.41 (m, 2H, ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.55 (t, 2H, CF2CH2), 4.03 (m, 2H, CH2S), 3.75 (m, 2H, CH2S), 2.27-2.43 (m, 4H, SCH2CH2), 1.42-1.99 (m, 29H, Adamantane) ¹⁹F-NMR (DMSO-d6, 376 MHz): δ (ppm) = −111.2. | 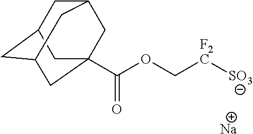 | ↑ | 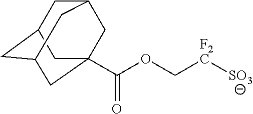 |
| 30 | PAG-30 | ¹H-NMR (DMSO-d6, 400 MHz): δ (ppm) = 8.41 (m, 2H, ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.40 (t, 2H, CH2), 4.20 (t, 2H, CH2), 4.03 (m, 2H, CH2S), 3.75 (m, 2H, CH2S), 2.27-2,43 (m, 4H, SCH2CH2), 2.05 (s, 2H, CH2), 1.42-1.99 (m, 29H, Adamantane), ¹⁹F-NMR (DMSO-d6, 376 MHz): δ (ppm) = −111.2. | 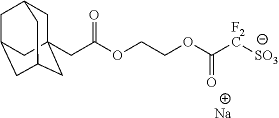 | ↑ | 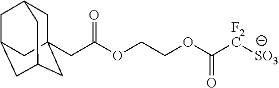 |

TABLE 12

| Example | Compound | NMR | Salt M⁺X⁻ | Cation | Anion |
|---|---|---|---|---|---|
| 31 | PAG-31 | ¹H-NMR (DMSO-d6, 400 MHz): δ (ppm) = 8.41 (m, 2H, ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.19 (s, 2H, CH2), 4.03 (m, 2H, CH2S), 3.75 (m, 2H, CH2S), 2.27-2.43 (m, 4H, SCH2CH2), 1.42-1.99 (m, 29H, Adamantane) ¹⁹F-NMR (DMSO-d6, 376 MHz): δ (ppm) = −77.7. | 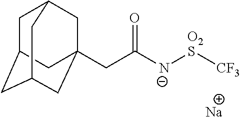 | ↑ | 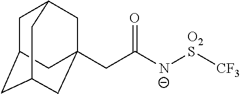 |
| 32 | PAG-32 | 1H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.77-2.81 (m, 1H, Cyclohexyl), 2.27-2.43 (m, 4H, SCH2CH2), 1.42-2.08 (m, 33H, Adamantane + Cyclohexyl), 1.07-1.33 (m, 5H, Cyclohexyl) 19F-NMR (DMSO-d6, 376 MHz): δ (ppm) = −74.7 | 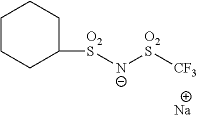 | ↑ | 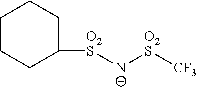 |
| 33 | PAG-33 | ¹H-NMR (DMSO-d6, 400 MHz): δ (ppm) = 8.41 (m, 2H, ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.27-2.43 (m, 4H, SCH2CH2), 1.42-1.99 (m, 29H, Adamantane) ¹⁹F-NMR (DMSO-d6, 376 MHz): δ (ppm) = −74.5. | 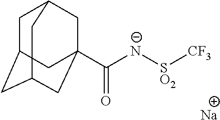 | ↑ | 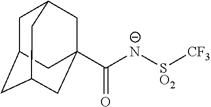 |

TABLE 13

| Example | Compound | NMR | Salt M⁺X⁻ | Cation | Anion |
|---|---|---|---|---|---|
| 34 | PAG-34 | ¹H-NMR (DMSO-d6, 400 MHz): δ (ppm) = 8.41 (m, 2H, ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.27-2.43 (m, 4H, SCH2CH2), 2.13 (m, 3H, Adamantane), 1.42-1.99 (m, 26H, Adamantane), ¹⁹F-NMR (DMSO-d6, 376 MHz): δ (ppm) = −69.2, −76.0, −112.9. | 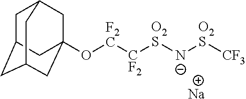 | ↑ | 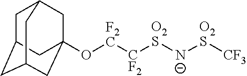 |
| 35 | PAG-35 | ¹H-NMR (DMSO-d6, 400 MHz): δ (ppm) = 8.41 (m, 2H, ArH), 8.12 (d, 1H, ArH), 7.51-7.96 (m, 9H, Ar—H + Naph), 7.19 (d, 1H, ArH), 5.23 (s, 2H, CH2), _4.95 (m, 1H, Adamantane), 4.03 (m, 2H, CH2S), 3.75 (m, 2H, CH2S), 2.27- 2.43 (m, 4H, SCH2CH2), 1.42-1.99 (m, 14H, Adamantane) ¹⁹F-NMR (DMSO-d6, 376 MHz): δ (ppm) = −80.5, −113.7. | 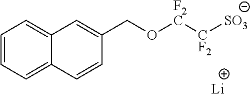 | ↑ | 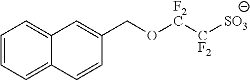 |
| 36 | PAG-36 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.27-2.43 (m, 4H, SCH2CH2), 2.09 (s, 3H, Adamantane), , 1.42-1.99 (m, 26H, Adamantane) ¹⁹F-NMR (DMSO-d6, 376 MHz): δ (ppm) = −70.1, −113.4. | 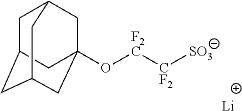 | ↑ | 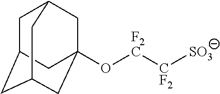 |

TABLE 14

| Example | Compound | NMR | Salt M⁺X⁻ | Cation | Anion |
|---|---|---|---|---|---|
| 37 | PAG-37 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.88 (d, 1H, CH), 2.66-2.74 (m, 1H, CH), 2.17-2.43 (m, 6H, SCH2CH2 + CH), 1.42-1.99 (m, 17H, Adamantane + CH + CH2), 1.22-1.29 (m, 2H, CH2), 1.03 (s, 3H, CH3), 0.71 (s, 3H, CH3) | 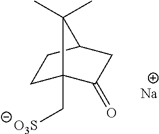 | ↑ | 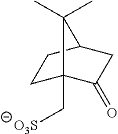 |
| 38 | PAG-38 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.27-2.43 (m, 4H, SCH2CH2), 1.42-1.99 (m, 14H, Adamantane) ¹⁹F-NMR (DMSO-d6, 376 MHz): δ (ppm) = −77.3, −111.5, −118.1, −122.4. | 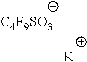 | ↑ | 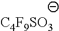 |
| 39 | PAG-39 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.27-2.43 (m, 4H, SCH2CH2), 1.42-1.99 (m, 14H, Adamantane) ¹⁹F-NMR (DMSO-d6, 376 MHz): δ (ppm) = −75.0 | 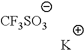 | ↑ | 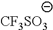 |

TABLE 15

| Ex- | Com- | | Salt | Cat- | |
|---|---|---|---|---|---|
| ample | pound | NMR | M⁺X⁻ | ion | Anion |
| 40 | PAG-40 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.03 (m, 2H, CH2S), 3.75 (m, 2H, CH2S), 2.27-2.43 (m, 4H, SCH2CH2), 1.42-1.99 (m, 14H, Adamantane) ¹⁹F-NMR (DMSO-d6, 376 MHz): δ (ppm) = −77.3, −112.5, −121.7. | 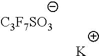 | ↑ | 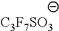 |
| 41 | PAG-41 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.83-5.92 (m, 1H, CH), 5.41 (dd, 1H, CH), 5.21- 5.23 (m, 3H, CH2 + CH), 4.95 (m, 1H, Adamantane), 4.45 (s, 2H, CH2), 4.03 (m, 2H, CH2S), 3.75 (m, 2H, CH2S), 2.27-2.43 (m, 4H, SCH2CH2), 1.42-1.99 (m, 14H, Adamantane), ¹⁹F-NMR (DMSO-d6, 376 MHz): δ (ppm) = −80.0, −113.0 | 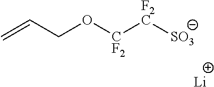 | ↑ | 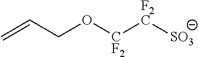 |
| 42 | PAG-42 | ¹H-NMR (DMSO-d6, 400 MHz): δ (ppm) = 8.41 (m, 2H, ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.49-4.51 (m, 2H, O—CH2), 4.30-4.32 (m, 2H, O—CH2), 4.03 (m, 2H, CH2S), 3.75 (m, 2H, CH2S), 2.27-2.43 (m, 6H, SCH2CH2 + COCH2), 1.42-1.99 (m, 16H, CH2 + Adamantane), 1.15-1.35 (m, 6H, CH2), 0.87 (t, 3H, CH3) ¹⁹F-NMR (DMSO-d6, 376 MHz): δ (ppm) = −106.7. | 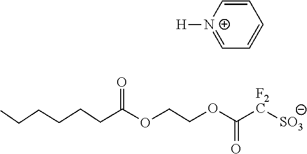 | ↑ | 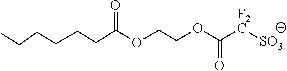 |

TABLE 16

| Example | Compound | NMR | Salt M⁺X⁻ | Cation | Anion |
|---|---|---|---|---|---|
| 43 | PAG-43 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.50-4.54 (m, 4H, OCH2CH2O), 4.03 (m, 2H, CH2S), 3.75 (m, 2H, CH2S), 3.57 (d, 1H, CH2SO2), 3.36 (sd, 1H, CH2SO2), 2.24-2.43 (m, 6H, CH2 + SCH2CH2), 1.42-2.07 (m, 19H, CH + CH2 + Adamantane) 1.04 (s, 3H, CH3), 0.84 (s, 3H, CH3) ¹⁹F-NMR (DMSO-d6, 376 MHz): δ (ppm) = −106.5 | 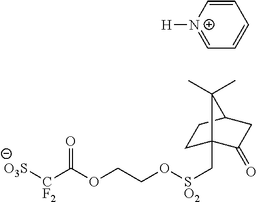 | ↑ | 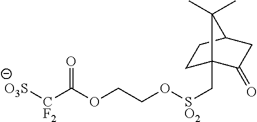 |
| 44 | PAG-44 | ¹H-NMR (DMSO-d6, 400 MHz): δ (ppm) = 8.41 (m, 2H ArH), 8.12 (d, 1H, ArH), 7.73-7.93 (m, 2H, ArH), 7.19 (d, 1H, ArH), 5.23 (s, 2H, CH2), 4.95 (m, 1H, Adamantane), 4.22 (s, 2H, CH2O), 4.03-4.05 (m, 4H, CH2CF2 + CH2S), 3.75 (m, 2H, CH2S), 3.13 (q, 6H, CH2CH3), 2.24-2.43 (m, 6H, SCH2CH2 + Adamantane), 1.42- 1.99 (m, 29H, CH3 + Adamantane), 1.20 (t, 9H, CH2CH3) ¹⁹F-NMR (DMSO-d6, 376 MHz): δ (ppm) = −111.0. | 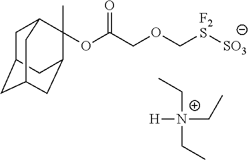 | ↑ | 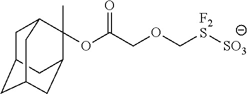 |

<Production of Positive Resist Composition>

The components shown in Table 17 were mixed together and dissolved to obtain positive resist compositions.

TABLE 17

| | (A) | (B) | (C) | (S) |
|---|---|---|---|---|
| Ex. 45 | (A)- 1 | (B)- 1 | (D)- 1 | (S)- 1 |
| | [100] | [10.96] | [1.45] | [2000] |
| Ex. 46 | (A)- 1 | (B)- 2 | (D)- 1 | (S)- 1 |
| | [100] | [10.96] | [1.45] | [2000] |
| Comp. | (A)- 1 | (B)- 3 | (D)- 1 | (S)- 1 |
| Ex. 1 | [100] | [8.88] | [1.45] | [2000] |
| Comp. | (A)- 1 | (B)- 4 | (D)- 1 | (S)- 1 |
| Ex. 2 | [100] | [11.28] | [1.45] | [2000] |
| Comp. | (A)- 1 | (B)- 5 | (D)- 1 | (S)- 1 |
| Ex. 3 | [100] | [10.22] | [1.45] | [2000] |
| Comp. | (A)- 1 | (B)- 6 | (D)- 1 | (S)- 1 |
| Ex. 4 | [100] | [9.40] | [1.45] | [2000] |
| Comp. | (A)- 1 | (B)- 7 | (D)- 1 | (S)- 1 |
| Ex. 5 | [100] | [8.43] | [1.45] | [2000] |

In Table 17, the reference characters indicate the following. Further, the values in brackets [ ] indicate the amount (in terms of parts by weight) of the component added.

(A)-1: a copolymer represented by chemical formula (A1-11-1) shown below with Mw=7,000 and Mw/Mn=1.70. In the formula, the subscript numerals shown to the bottom right of the parentheses ( ) indicate the percentage (mol %) of the respective structural units within the copolymer.

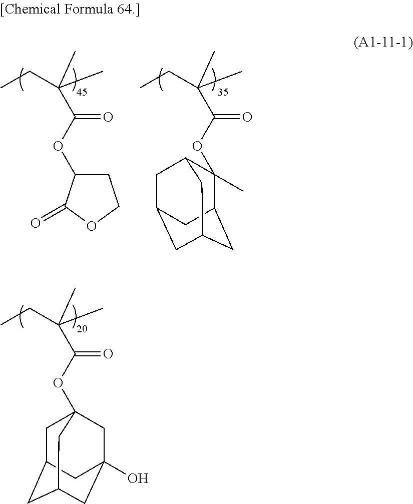

(B)-1: the aforementioned compound PAG-1

(B)-2: the aforementioned compound PAG-23

(B)-3: a compound represented by formula (B)-3 shown below

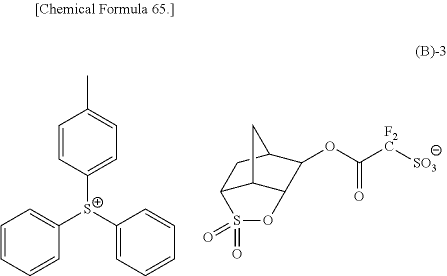

(B)-4: a compound represented by formula (B)-4 shown below

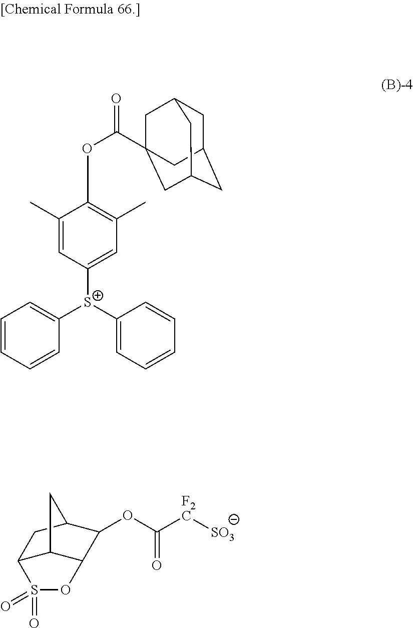

(B)-5: a compound represented by formula (B)-5 shown below

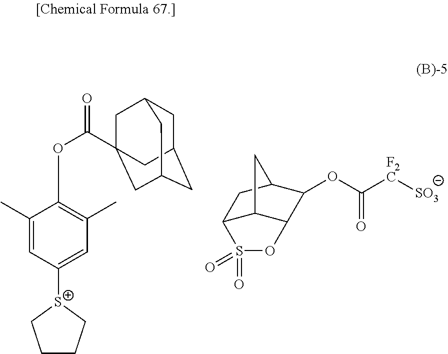

(B)-6: a compound represented by formula (B)-6 shown below

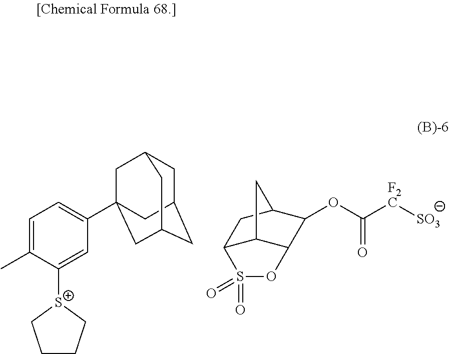

(B)-7: a compound represented by formula (B)-7 shown below

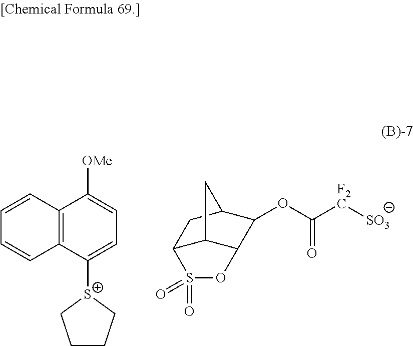

(D)-1: triethanolamine triacetate

(S)-1: a mixed solvent of PGMEA/PGME/cyclohexanone=45/30/25 (weight ratio)

[Resolution and Sensitivity]

An organic anti-reflection film composition (product name: ARC29A, manufactured by Brewer Science Ltd.) was applied to an 8-inch silicon wafer using a spinner, and the composition was then baked on a hot plate at 205° C. for 60 seconds, thereby forming an organic anti-reflection film having a film thickness of 82 nm. Then, the resist composition was applied to the anti-reflection film using a spinner, and was then prebaked (PAB) on a hotplate at 110° C. for 60 seconds and dried, thereby forming a resist film having a film thickness of 150 nm.

Subsequently, the resist film was selectively irradiated with an ArF excimer laser (193 nm) through a mask pattern (6% half tone mask), using an ArF exposure apparatus NSR-S302 (manufactured by Nikon Corporation, NA (numerical aperture)=0.60, 2/3 annular illumination). Thereafter, a post exposure bake (PEB) treatment was conducted at 110° C. for 60 seconds, followed by development for 30 seconds at 23° C. in a 2.38% by weight aqueous solution of tetramethylammonium hydroxide (TMAH) (product name: NMD-3; manufactured by Tokyo Ohka Kogyo Co., Ltd.). Then, the resist was washed for 30 seconds with pure water, followed by drying by shaking.

As a result, in each of the examples, a 1:1 line and space resist pattern (L/S pattern) having a line width of 120 nm and a pitch of 240 nm was formed on the resist film. The optimum exposure dose Eop (mJ/cm²) with which the pattern was formed, i.e., sensitivity, was determined. The results are shown in Table 18.

[Evaluation of Line Width Roughness (LWR)]

With respect to each of the L/S patterns formed with the above Eop and having a line width of 120 nm and a pitch of 240 nm, the line width at 5 points in the lengthwise direction of the line were measured using a measuring scanning electron microscope (SEM) (product name: S-9220, manufactured by Hitachi, Ltd.; acceleration voltage: 800V), and from the results, the value of 3 times the standard deviation s (i.e., 3s) was calculated as a yardstick of LWR. The results are shown in Table 18. The smaller this value is, the lower the level of roughness of the line width, indicating that an L/S pattern with a uniform width was obtained.

[Evaluation of EL Margin]

The exposure dose with which an L/S pattern having a dimension of the target dimension (line width: 120 nm) ±5% (i.e., 114 nm to 126 nm) was formed was determined, and the EL margin (unit: %) was determined by the following formula. The results are shown in Table 18.

EL margin (%)=(|E1−E2|/Eop)×100

In the formula, E1 represents the exposure dose (mJ/cm²) for forming an L/S pattern having a line width of 114 nm, and E2 represents the exposure dose (mJ/cm²) for forming an L/S pattern having a line width of 126 nm.

[Evaluation of Mask Error Factor (MEF)]

With the above Eop, L/S patterns were formed using a mask targeting a line width of 130 nm and a pitch of 260 nm, and a mask targeting a line width of 120 nm and a pitch of 260 nm, and the MEF value was calculated by the following formula. The results are shown in Table 18.

MEF=|CD₁₃₀−CD₁₂₀|/MD₁₃₀−MD₁₂₀|

In the formula, CD₁₃₀ and CD₁₂₀ represent the respective line widths (nm) of the actual L/S patterns respectively formed using the mask pattern targeting a line width of 130 nm and the mask pattern targeting a line width of 120 nm. MD₁₃₀ and MD₁₂₀ represent the respective target line widths (nm), meaning MD₁₃₀=130, and MD₁₂₀=120. A MEF value closer to 1 indicates that a resist pattern faithful to the mask pattern was formed.

TABLE 18

| | Eop | EL | | LWR |
|---|---|---|---|---|
| | (mJ/cm²) | (%) | MEF | (nm) |
| Ex. 45 | 73.93 | 12.17 | 2.45 | 7.90 |
| Ex. 46 | 40.33 | 12.33 | 2.49 | 8.30 |
| Comp. Ex. 1 | 24.79 | 8.05 | 2.41 | 11.17 |
| Comp. Ex. 2 | 45.58 | 8.07 | 2.54 | 15.10 |
| Comp. Ex. 3 | 60.94 | 10.85 | 2.51 | 8.20 |
| Comp. Ex. 4 | 54.31 | 10.57 | 1.90 | 11.00 |
| Comp. Ex. 5 | 49.62 | 11.08 | 2.49 | 13.80 |

As shown in Table 18, the resist compositions of Examples 45 and 46 using the compound of the present invention as the acid-generator component (B) exhibited excellent LWR and EL, as compared to the resist compositions of the comparative examples.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A resist composition comprising a base component (A) which exhibits changed solubility in an alkali developing solution under action of acid and an acid-generator component (B) which generates acid upon exposure,
the acid-generator component (B) comprising an acid generator (B1) comprised of a compound represented by general formula (b1) shown below: 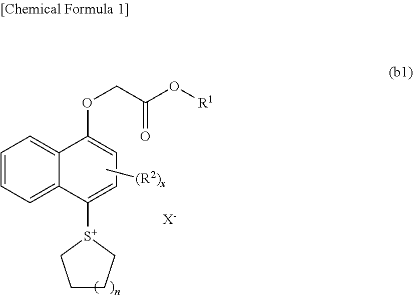
wherein R¹ represents a hydrogen atom, a linear, branched or cyclic alkyl group of 1 to 10 carbon atoms or a heterocyclic group of 1 to 10 carbon atoms; R² represents a linear or branched alkyl group of 1 to 10 carbon atoms; x represents an integer of 0 to 6; n represents an integer of 0 to 3; and X⁻ represents an anion.

2. The resist composition according to claim 1, wherein X⁻ in general formula (b1) is at least one anion selected from the group consisting of compounds represented by any one of general formulas (1) to (4) shown below: 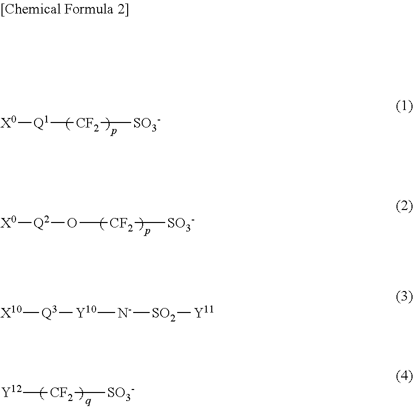
wherein each X⁰ independently represents a hydrocarbon group of 1 to 30 carbon atoms which may have a substituent; Q¹ represents a single bond or a divalent linking group containing a carbonyl group; each p independently represents an integer of 0 to 3; Q² represents a single bond or an alkylene group; X¹⁰ represents a hydrocarbon group of 1 to 30 carbon atoms which may have a substituent; Q³ represents a single bond or a divalent linking group; Y¹⁰ represents —C(═O)— or —SO₂—; Y¹¹ represents an alkyl group of 1 to 10 carbon atoms which may have a substituent or a fluorinated alkyl group of 1 to 10 carbon atoms which may have a substituent; Y¹² represents a cyclic alkyl group of 4 to 20 carbon atoms which may have an oxygen atom (═O) as a substituent; and q represents 0 or 1.

3. The resist composition according to claim 2, wherein X⁻ in general formula (b1) is an anion represented by general formula (1-1-12-1) shown below:

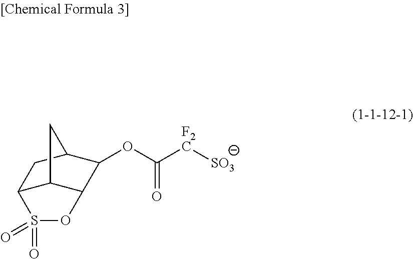

4. A method of forming a resist pattern, comprising forming a resist film on a substrate using a resist composition according to claim 1, subjecting the resist film to exposure, and subjecting the resist film to alkali developing to form a resist pattern.

5. A compound represented by general formula (b1) shown below: 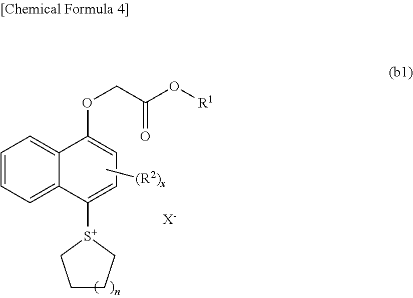
wherein R¹ represents a hydrogen atom, a linear, branched or cyclic alkyl group of 1 to 10 carbon atoms or a heterocyclic group of 1 to 10 carbon atoms; R² represents a linear or branched alkyl group of 1 to 10 carbon atoms; x represents an integer of 0 to 6; n represents an integer of 0 to 3; and X⁻ represents an anion.

6. The compound according to claim 5, wherein X⁻ in general formula (b1) is at least one anion selected from the group consisting of compounds represented by any one of general formulas (1) to (4) shown below: 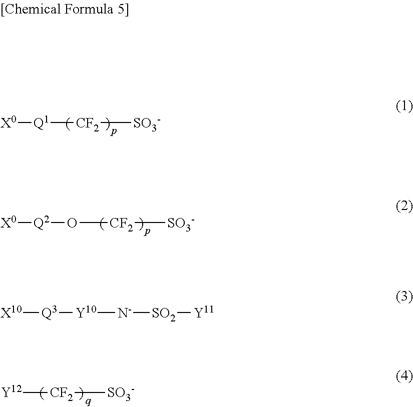
wherein each X⁰ independently represents a hydrocarbon group of 1 to 30 carbon atoms which may have a substituent; Q¹ represents a single bond or a divalent linking group containing a carbonyl group; each p independently represents an integer of 0 to 3; Q² represents a single bond or an alkylene group; X¹⁰ represents a hydrocarbon group of 1 to 30 carbon atoms which may have a substituent; Q³ represents a single bond or a divalent linking group; Y¹⁰ represents —C(═O)— or —SO₂—; Y¹¹ represents an alkyl group of 1 to 10 carbon atoms which may have a substituent or a fluorinated alkyl group of 1 to 10 carbon atoms which may have a substituent; Y¹² represents a cyclic alkyl group of 4 to 20 carbon atoms which may have an oxygen atom (═O) as a substituent; and q represents 0 or 1.

7. An acid generator comprising the compound according to claim 5.
